(19) United States
(12) Patent Application Publication
(10) Pub. No.: US 2008/0057055 A1
(43) Pub. Date: Mar. 6, 2008

(54) OLD-35 as an inflammatory agent

(76) Inventors: Paul B. Fisher, Scarsdale, NY (US); Devanand Sarkar, Elmsford, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/784,096

(22) Filed: Apr. 5, 2007

Related U.S. Application Data
(63) Continuation of application No. PCT/US05/36409, filed on Oct. 7, 2005.
(60) Provisional application No. 60/616,774, filed on Oct. 7, 2004.

(51) Int. Cl. A61K 39/395; A01K 67/033; A61P 19/02; C07H 21/00; G01N 33/50; C12Q 1/68; A61P 25/28; A61K 31/7088

(52) U.S. Cl. 424/130.1; 435/6; 436/94; 514/44; 536/23.1; 800/12; 800/9

(57) ABSTRACT

The present invention relates to the discovery that OLD-35, at least in part through the generation of reactive oxygen species, induces a number of inflammatory cytokines and promotes nuclear translocation and binding of the transcriptional activator NF-κB. Accordingly, the present invention provides for assay systems (which either utilize the old-35 promoter or the old-35 gene) that may be used to identify new anti-inflammatory agents; model systems of inflammation based on over-expression of the old-35 gene in cells and tissues (including specific model systems for arthritis, atherosclerosis and Alzheimer's disease); methods and kits for diagnosing old-35 associated inflammatory conditions, and methods of treatment and anti-inflammatory compositions that utilize agents that antagonize OLD-35 activity.

### CROSS-REFERENCE TO RELATED APPLICATIONS

This application claims priority to U.S. Provisional Patent Application No. 60/616,774 filed Oct. 7, 2004 which is incorporated by reference in its entirety herein.

### 1. INTRODUCTION

The present invention relates to methods of modulating inflammation by altering the expression and/or activity of OLD-35, a protein originally identified by its association with senescence and terminal cell differentiation. The invention is based on the discovery that OLD-35, at least in part through the generation of reactive oxygen species, induces a number of inflammatory cytokines and promotes nuclear translocation and binding of the transcriptional activator NF-κB. The present invention further provides for active portions of OLD-35 which may be used therapeutically.

### 2. BACKGROUND OF THE INVENTION

Human polynucleotide phosphorylase (hPNPase^{old-35}, referred to as “OLD-35” herein) was identified as a previously unknown gene, old-35, by an overlapping pathway screening (OPS) approach due to its upregulation during cellular differentiation and senescence (1). Old-35, a 3′, 5′ exoribonuclease, is a predominantly type I interferon-inducible gene highly evolutionary conserved in plants, prokaryotes and eukaryotes having similar domain structures and functional properties in all species (1-3). Its expression is also augmented in senescent progeroid fibroblasts in comparison to young fibroblasts (1). Overexpression of old-35 via a replication incompetent adenovirus (Ad.hPNPase) in HO-1 human melanoma cells and in normal human melanocytes (NHuMel) produced a senescent phenotype characterized by growth arrest in the G₁ phase, increased Senescence Associated β-galactosidase activity, decreased telomerase activity and defined senescence-associated gene expression changes (4). These profound alterations induced by old-35 suggest an essential role in controlling senescence and differentiation through its property as an exoribonuclease by targeting selective RNA degradation. This hypothesis is supported by the observations that old-35 induces specific degradation of c-myc mRNA and that overexpression of c-myc partially protects HO-1 cells from old-35-induced growth arrest (4).

Oxidative stress is a potential mediator of in vitro replicative and premature senescence and in vivo aging (5). The free radical theory of aging, as proposed by Harman, states that endogenous reactive oxygen species (“ROS”) are generated in cells resulting in a pattern of cumulative damage (6). Oxidative damage can be measured by formation of 8-oxo-2′-deoxyguanosine (oxo⁸dG) in DNA or free 8-oxoguanine base (oxo⁸Gua) release by cells (7). Replicative senescent cells contain approximately 30% more oxo⁸dG in their DNA and produce four times more free oxo⁸Gua bases (8). Tissues from aged individuals or aged experimental animals accumulate oxidative damage in their DNA, protein and lipids (9). Moreover, repeated subcytotoxic oxidative damage can induce premature senescence in multiple cell types such as fibroblasts, keratinocytes, melanocytes or umbilical vascular endothelial cells (10) and treatment with a cell-permeable anti-oxidant or culturing cells in a reduced ambient oxygen content can reverse growth arrest of fibroblasts induced by expression of activated Ras (11). ROS comprise a variety of diverse chemical species including superoxide anions, hydroxyl radicals and hydrogen peroxide (5). Although cytosolic enzymes such as NADPH oxidases contribute to the generation of ROS, the majority of intracellular ROS production generates from mitochondria (5). Additionally, aged animals contain defective mitochondria and can produce higher levels of ROS than their young counterparts (12).

A prominent mechanism by which ROS modulates diverse intracellular molecular processes is by regulating the activity of transcription factors, most notably nuclear factor (NF)-κB (13). As a corollary to increased ROS generation during the aging process, increased NF-κB DNA binding activity has been documented in multiple tissues of aged animals compared with young animals (14-21). In resting cells, NF-κB resides in the cytoplasm in an inactive form bound to an inhibitory protein known as IκB (22). Upon receiving a stimulus, such as ROS, IκB kinase (IKK) is activated which in turn phosphorylates IκB proteins making them susceptible to ubiquitin-proteosome-mediated degradation (23, 24). The destruction of IκB unmasks the nuclear localization signal of NF-κB, leading to nuclear translocation and regulation of gene transcription by binding to the decameric motif, “GGGRNNYYCC” in the promoters of target genes (25). Presently, five mammalian NF-κB family members, NF-κB1 (p50/p105), NF-κB2 (p52/p100), p65 (RelA), RelB and c-Rel, have been identified and cloned (26). The most abundant activated form of NF-κB is a heterodimer composed of a p50 and a p65 subunit that functions predominantly as a transcriptional activator.

### 3. SUMMARY OF THE INVENTION

The present invention relates to the discovery that OLD-35, at least in part through the generation of reactive oxygen species, induces a number of inflammatory cytokines and promotes nuclear translocation and binding of the transcriptional activator NF-κB. Accordingly, the present invention provides for assay systems (which either utilize the old-35 promoter or the old-35 gene) that may be used to identify new anti-inflammatory agents; model systems of inflammation based on over-expression of the old-35 gene in cells and tissues (including specific model systems for arthritis, atherosclerosis and Alzlieimer's disease); methods and kits for diagnosing old-35 associated inflammatory conditions, and methods of treatment and anti-inflammatory compositions that utilize agents that antagonize OLD-35 activity. The present invention further relates to portions of OLD-35 which exhibit PNPase activity. Such portions may be used therapeutically to inhibit cell proliferation (for example, in the context of malignancy) or to act as “vaccines” to inhibit inflammation

### 4. BRIEF DESCRIPTION OF THE FIGURES

FIG. 1A-D. OLD-35 is localized in the mitochondria and Ad.hPNPase infection results in the generation of ROS. (A.) HeLa cells were either uninfected or infected with Ad.vec or Ad.hPNPase at a m.o.i. of 50 pfu/cell. The expression of OLD-35 was analyzed 2 days post-infection by Western blot analysis. (B.) HeLa cells were infected with Ad.hPNPase at a m.o.i. of 50 pfu/cell. Subcellular localization of OLD-35 was analyzed by staining with MitoTracker and anti-OLD-35-antibody. (C.) HeLa cells were infected with Ad.vec or Ad.hPNPase at a m.o.i. of 50 pfu/cell and untreated or treated with 20 mM NAC 2 hr post-infection. The generation of ROS was measured by flow cytometry as described in Materials and Methods (below). The panels represent flow cytometry histograms at 24 h post-infection. (D.) Graphical representation of quantification of ROS containing cells at indicated time periods after infection. The data represent the mean ± S.D. of three independent experiments.

FIG. 2A-B. OLD-35 augments NF-κB reporter gene activity by generating ROS. (A.) HeLa cells were infected with the indicated adenoviruses, transfected with the indicated plasmids and luciferase activity was measured as described in materials and methods. (B.) HeLa cells were infected with either Ad.vec or Ad.hPNPase at a m.o.i. of 50 pfu/cell and 12 hr after infection transfected with the plasmid 3 KB-Luc. The cells were pretreated with NAC or Tiron 2 hr before transfection. Luciferase assay was carried out 48 hr post-transfection. Lanes 1-3: Ad.vec; 2: +NAC (20 mM); 3: +Tiron (4.5 mM). 4-10: Ad.hPNPase; 5: +NAC (5 mM); 6: +NAC (10 mM); 7: +NAC (20 mM); 8: +Tiron (0.9 mM) 9: +Tiron (2.25 mM); 10: +Tiron (4.5 mM). The luciferase activity was normalized by β-galactosidase activity. The data represent mean ± S.D. of three independent experiments each performed in triplicates.

FIG. 3A-D. Ad.hPNPase infection increases NF-κB DNA binding by generating ROS. (A.) HeLa cells were infected as in FIG. 1A or with Ad.vec at 50 m.o.i. or with Ad.hPNPase at 1, 5, 10 or 50 m.o.i. and NF-κB DNA binding was analyzed in the nuclear extracts of the cells by EMSA at the indicated time points. (B.) HeLa cells were infected with Ad.vec or Ad.hPNPase at 50 m.o.i. and NF-κB DNA binding was analyzed 2 days post-infection. Cold WT: unlabeled consensus NF-κB probe; Cold MUT: unlabeled mutated NF-κB probe. Supershift analysis was carried out with the indicated antibodies. *: supershifted band by anti-p50 antibody; ** supershifted band by anti-p65 antibody. (C.) HeLa cells were infected with the indicated adenoviruses at 50 m.o.i. and NF-κB DNA binding was analyzed 2 days post-infection. D. HeLa cells were infected with Ad.vec or Ad.hPNPase at 50 m.o.i. and treated or not with 20 mM NAC. NF-κB DNA binding was analyzed 2 days post-infection.

FIG. 4A-C. Ad.hPNPase infection results in IκBcc degradation and nuclear translocation of p65 by generating ROS. HeLa cells were infected as in FIG. 1A and the expressions of the indicated proteins were analyzed in (A.) cytoplasmic extract and (B.) nuclear extract by Western blot analysis at the indicated time points. (C.) HeLa cells were infected as in FIG. 1A and treated with 20 mM NAC. The expressions of the indicated proteins were analyzed by Western blot analysis 2 days post-infection.

FIG. 5A-C. Ad.hPNPase infection induces the expression of IL-6 and IL-8 by generating ROS. (A.) HeLa cells were infected as in FIG. 1A and RT-PCR was performed for the indicated genes. Lanes 1, 3 and 5: Ad.vec; 2, 4 and 6: Ad.hPNPas; lanes 1 and 2 are 1 day, 3 and 4 are 2 days and 5 and 6 are 3 days post-infection. (B) and (C.) HeLa cells were infected as in FIG. 1A and treated with 20 mM NAC. The levels of secreted IL-6 (B) and IL-8 (C) in culture supernatants were analyzed by ELISA. The data represent mean ± S.D. of two independent experiments each performed in triplicates.

FIG. 6A-C. Ad.hPNPase infection induces pro-inflammatory cytokines. HeLa cells were infected as in FIG. 1A and the levels of secreted cytokines in culture supernatants were analyzed by TranSignal™ human cytokine antibody array. (A.) Schematic representation of the layout of the blotted membranes. The grids marked by asterisks indicate the cytokines which were induced by OLD-35. (B.) Membrane incubated with culture supernatants from Ad.vec-infected cells. (C.) Membrane incubated with culture supernatants from Ad.hPNPas-infected cells.

FIG. 7A-B. Nucleic acid sequence of old-35 promoter (SEQ ID NO:1). The end of the promoter and the beginning of the transcribed gene is indicated by underlining of the gene sequence. The portion of the promoter incorporated in p2000 is set forth in boldface print. Parts (A) and (B) of the figure contain consecutive sequence.

FIG. 8. Graphical representation of various deletions in the old-35 promoter. The construct sizes are not drawn to scale. Numbers on the right describe the approximate construct sizes.

FIG. 9. Graphical representation of the cloned 2-kb old-35 promoter. The immediate 400-bp sequence of the old-35 promoter (SEQ ID NO:2) including the transcription initiation site is shown (the arrow).Additional sites that may be related to IFN signaling or to the constitutive activity of the old-35 promoter are underlined and bolded. The numbers below the elements indicate the initial bp at which the element is located.

FIG. 10. Sequences of the consensus ISRE (SEQ ID NO: 3), the old-35 ISRE (SEQ ID NO:4).

FIG. 11A-B. Sequence of (A) old-35 cDNA (SEQ ID NO:5) and (B) OLD-35 protein (SEQ ID NO:6).

FIG. 12A-E. The RPH domain of hPNPase^{old-35} is required for induction of senescence in HO-1 cells. A. Schematic representation of the domain structure of the hPNPase^{OLD-35} protein and the different deletion mutants. The numbers represent the amino acid numbers. The grey box is the mitochondrial localization signal, white boxes are the RPH domains and the hatched boxes are the RNA binding domains. B. HO-1 cells were either infected with the indicated Ad at an m.o.i. of 50 pfu/cell or treated with interferon-β at a dose of 1000 units/ml and the levels of the indicated proteins were analyzed by Western blot analysis 48 h later. C. HO1 cells were either uninfected or infected with Ad.vec at an m.o.i. of 50 pfu/cell or with the indicated Ad at an m.o.i. of 10, 20 or 50 pfu/cell and colony formation assays were determined as described in experimental procedures. The data represents mean ± S.D. and is a representation of three independent experiments each performed in triplicates. D. Microphotograph of HO-1 cells infected with the indicated Ad at an m.o.i. of 50 pfu/cell 4 days post-infection. The white arrows indicate the large, flattened cells that stain for SA-β-gal. E. Quantification of SA-β-gal-positive cells. At least 1,000 cells were counted for each group. The data represents mean ± S.D. of three independent experiments.

FIG. 13A-D. hPNPase^{old-35} and its RPH-containing deletion mutants inhibit cell cycle at the G₁ phase. HO-1 cells were infected with the indicated Ad at an m.o.i. of 50 pfu/cell and cell cycle was analyzed on day 1, 2 and 3 post-infection. A. Flow cytometry histogram of cells infected with the indicated Ad 3 days post-infection. B-D. Graphical representation of the percentage of cells in the G₁ (B), S(C) and G₂+M (D) phases of the cell cycle. The data represents mean ± S.D. of three independent experiments.

FIG. 14A-C. Regulation of expression of cell cycle regulatory proteins by hPNPase^{old-35} and its deletion mutants. A. HO-1 cells were infected with the indicated Ad at an m.o.i. of 50 pfu/cell for 3 days and the expression of the indicated proteins in the cell lysates were analyzed by Western blot analysis. B. HO-1 cells were treated as in A and CDK2 activity was assayed using Histone H1 as substrate as described in experimental procedures. The expression level of CDK2 in 5% of the input used for the CDK2 activity assay was determined by Western blot analysis. C. HO-1 cells were infected with Ad.hPNPase^{old-35} at an m.o.i. of 50 pfu/cell for 2 days and immunofluorescence studies were performed to analyze the expression of Ad.hPNPase^{old-35} and p27^{KIP-1} as described in materials and methods.

FIG. 15A-D. The RPH domain of hPNPase^{old-35} is required for c-myc downregulation. A. HO-1 cells were treated as in FIG. 3A and the expression of the indicated mRNAs were analyzed by Northern blot analysis. B. Graphical representation of the c-myc/GAPDH mRNA level in the indicated treatment groups. The data represents mean ± S.D. of three independent experiments. C. HO-1 cells were treated as in FIG. 14A and the expression of the indicated proteins were analyzed by Western blot analysis. D. HO-1 cells were either transfected with an empty vector or with a c-myc expression vector and 36 h later infected with the indicated Ad at an m.o.i. of 50 pfu/cell and colony formation assays were performed. The data represents the mean ± S.D. and is a representation of three independent experiments each performed in triplicates.

FIG. 16A-C. The RPH domain of hPNPase^{old-35} is required for in vitro degradation of c-myc mRNA. A. Representation of the in vitro translated products from the indicated plasmids documenting their authenticity. B. In vitro degradation assays were performed as described in experimental procedures. The expression of c-myc, GADD34 and GAPDH mRNAs were detected by Northern blot analysis. C. Quantification of c-myc/GAPDH mRNA levels in the different groups analyzed in panel B. The data represents the mean ± S.D. of three independent experiments.

FIG. 17. hPNPase^{old-35} downregulates Myc in cells in the G₁ phase of the cell cycle. HO-1 cells were transfected with either empty vector or a c-myc expression plasmid and then infected with either Ad.vec or Ad.hPNPase^{old-35} at an m.o.i. of 50 pfu/cell for 2 days. The cells were sorted as described in materials and methods and the expression levels of the indicated proteins in cells of different phases of cell cycle were analyzed by Western blot analysis. G₂ represents cells in G₂+M phases.

FIG. 18A-B. Distribution of hPNPase^{OLD-35} and its deletion mutants in mitochondria and cytoplasmic compartments. A. HO-1 cells were infected with the indicated Ad and cell fractionation was performed 36 h later. The expressions of the indicated proteins were detected by Western blot analyses. The asterisks indicate the specific bands in the corresponding lanes. B. HO-1 cells were infected with the indicated Ad and 36 h later loaded with MitoTracker and stained with anti-HA antibody and visualized using a confocal laser scanning microscope.

FIG. 19A-C. Treatment with IFN-β upregulates hPNPase^{old-35} and downregulates c-myc mRNAs and proteins. A. The various cell lines were treated with IFN-β(1000 U/ml) for the indicated periods of time and the expression of hPNPase^{old-35}, c-myc and GAPDH mRNAs were analyzed by Northern blot analysis. B. The indicated cells were treated with IFN-β (1000 U/ml) for 1 and 2 days or with 1, 10, 100 or 1000 U/ml of IFN-β for 2 days and the expression of hPNPase^{OLD-35}, Myc and EF1-α proteins were analyzed by Western blot analysis. C. 2fTGH human fibrosarcoma cells and its four variants, U1A (Tyk2-), U3A (STAT1-), U4A (JAK1-) and U5A (IFN2AR-), were treated with IFN-β (1000 U/ml) for 2 days and the expression of the indicated proteins were analyzed by Western blot analysis.

FIG. 20A-B. IFN-β treatment inhibits growth and colony formation of human melanoma cells and immortalized human melanocytes. A. The different cell types were treated with the indicated concentrations of IFN-β and cell viability was assessed by standard MTT assay on day 3 and 6 post-treatment. The data represents the mean ± S.D. of three independent experiments each performed in octaplicates. B. The different cells were plated in 6-cm dish at a density of 1,000 cells/dish and then treated with the indicated concentrations of IFN-β. Colonies were counted after 2 weeks. At least 4 dishes were used for each data point in each experiment. The data represents the mean ± S.D. of two independent experiments.

FIG. 21A-B. HO-1 clones expressing hPNPase^{old-35} siRNA are resistant to IFN-β-mediated c-myc downregulation. A. Parental HO-1 cells, HO-1 clones expressing hPNPase^{old-35} siRNA (old-35-si clone 1, clone 4 and clone 5) and HO-1 clones expressing control siRNA (control-si clone 1) were treated with IFN-β (1000 U/ml) for 2 days and the expression of hPNPase^{OLD-35}, Myc, MDA-5 and EF1-α proteins were analyzed by Western blot analysis. B. For analysis of half-life of c-myc mRNA, cells were either untreated or treated with IFN-β (1000 U/ml) for 24 h and then exposed to Actinomycin D (5 μg/ml) for 0.5, 1, 2, 4 and 8 h after which the cells were harvested for total RNA extraction and Northern blot analysis using the indicated probes.

FIG. 22A-B. HO-1 clones expressing hPNPase^{old-35} siRNA are resistant to IFN-β-mediated growth inhibition that can be reversed by c-myc siRNA. A. HO-1 cells were transfected with either control siRNA or c-myc siRNA and the expression of Myc and EF1-α proteins were analyzed by Western blot analysis. B. HO-1 cells, HO-1 clones expressing hPNPase^{old-35} siRNA (old-35-si clone 1, clone 4 and clone 5) and HO-1 clones expressing control siRNA (control-si clone 1) were either mock-transfected (control) or transfected with control siRNA or c-myc siRNA and then treated with the indicated concentrations of IFN-β and cell viability was assessed by standard MTT assay on day 3 and 6 post-treatment. The data represents the mean ± S.D. of three independent experiments each performed in octaplicates.

FIG. 23. HO-1 clones expressing hPNPase^{old-35} siRNA are resistant to IFN-β-mediated colony formation inhibition that can be reversed by c-myc siRNA. HO-1 cells, HO-1 clones expressing hPNPase^{old-35} siRNA (old-35-si clone 1, clone 4 and clone 5) and HO-1 clones expressing control siRNA (control-si clone 1) were either mock-transfected (control) or transfected with control siRNA or c-myc siRNA and then treated with the indicated concentrations of IFN-β and colony formation assay was performed. Colonies were counted after 2 weeks. At least 4 dishes were used for each data point in each experiment. The data represents the mean ± S.D. of two independent experiments.

FIG. 24. HO-1 clones expressing hPNPase^{old-35} siRNA are resistant to IFN-β-induced G₁ cell cycle arrest and apoptosis. HO-1 cells, HO-1 clones expressing hPNPase^{old-35} siRNA (old-35-si clone 1 and clone 4) and HO-1 clones expressing control siRNA (control-si clone 1) were treated with IFN-β (1000 U/ml) and cell cycle was analyzed by flow cytometry on day 1 and 3 post-treatment.

FIG. 25A-C. HO-1 clones overexpressing Myc are resistant to IFN-β-mediated growth and colony formation inhibition. A. Myc and EF1-α expressions were analyzed by Western blot analysis in control HO-1 clones (HO-1-Hygro-clone-1 and HO-1-Hygro-clone-4) and Myc overexpressing clones (HO-1-Myc-clone-1 and HO-1-Myc-clone-2) treated or not with IFN-β (1000 U/ml) for 2 days. B. Control HO-1 clones (HO-1-Hygro-clone-1 and HO-1-Hygro-clone-4) and Myc overexpressing clones (HO-1-Myc-clone-1 and HO-1-Myc-clone-2) were treated with the indicated concentrations of IFN-β and colony formation assays were performed. Colonies were counted after 2 weeks. At least 4 dishes were used for each data point in each experiment. The data represents the mean ± S.D. of two independent experiments. C. Control HO-1 clones (HO-1-Hygro-clone-1 and HO-1-Hygro-clone-4) and Myc overexpressing clones (HO-1-Myc-clone-1 and HO-1-Myc-clone-2) were treated with the indicated concentrations of IFN-β and cell viability was assessed by standard MTT assay on day 3 and 6 post-treatment. The data represents the mean ± S.D. of three independent experiments each performed in octaplicates.

FIG. 26. A schematic model of regulation of hPNPase^{old-35} and c-myc by IFN-β. Binding of IFN-α/β to the cognate receptors IFNAR1 and IFNAR2 results in cross-phosphorylation and activation of TYK2 and JAK1 with subsequent phosphorylation of STAT1 and STAT2. Phosphorylated STAT1/STAT2 heterodimer translocates to the nucleus, associates with p48 to form the ISGF3 complex that binds to the promoter of hPNPase^{old-35} and upregulates its transcription. hPNPase^{OLD-35} protein enters into cytoplasm and binds and degrades c-myc mRNA by its 3′, 5′ exoribonuclease activity. Downregulation of c-myc results in cell cycle arrest in G₁ phase with subsequent apoptosis.

FIG. 27. Table 1. HO-1, WM-35, MeWo and FM-516 cells were treated with IFN-β (1000 U/ml) and cell cycle was analyzed by flow cytometry on day 1, 2 and 3 post-treatment. Bold IFN-β-treated data points marked with asterisks indicate significant differences (p<0.01) from the control data points.

FIG. 28. Table 2. HO-1 cells, HO-1 clones expressing hPNPase^{old-35} siRNA (old-35-si clone 1, clone 4 and clone 5) and HO-1 clones expressing control siRNA (control-si clone 1) were either mock-transfected (control) or transfected with control siRNA or c-myc siRNA and then treated with IFN-β (1000 U/ml) and cell cycle was analyzed by flow cytometry on day 1, 2 and 3 post-treatment. Bold data points marked with asterisks indicate significant differences (p<0.01) from the control data points.

FIG. 29. Table 3. Control HO-1 clones (HO-1-Hygro-clone-1 and HO-1-Hygro-clone-4) and Myc overexpressing clones (HO-1-Myc-clone-1 and HO-1-Myc-clone-2) were treated with IFN-β (1000 U/ml) and cell cycle was analyzed by flow cytometry on day 1, 2 and 3 post-treatment. Bold IFN-β-treated data points marked with asterisks indicate significant differences (p<0.01) from the control data points.

### 5. DETAILED DESCRIPTION OF THE INVENTION

For clarity and not by way of limitation, the detailed description of the invention is divided into the following subsections:
(i) assay systems (which either utilize the old-35 promoter or the old-35 gene);
(ii) model systems of inflammation;
(iii) diagnostic methods and kits;
(iv) methods of treatment and anti-inflammatory compositions;
(v) active subregions of OLD-35; and
(vi) use of OLD-35 variants.

Note that the old-35 gene and corresponding nucleic acids (genomic DNA, cDNA, mRNA, antisense RNA, RNA-i, etc.) is designated by all lower case letters, the protein is designated by all capital letters (i.e., OLD-35) and the gene and protein are collectively designated by a capitalized first letter only (i.e., Old-35).

The term “antibody,” as used herein, refers to a complete imnunoglobulin molecule as well as fragments and derivatives thereof, single-chain antibodies, and any other functional equivalents. The antibody may be polyclonal or monoclonal, chimeric, and biologically or chemically produced.

5.1. Assay Systems

The present invention provides for assay methods and systems that may be used to identify agents that modulate the transcription of old-35, its translation into protein and/or its biological activity. The agents may be molecules that occur in nature (e.g., a protein that binds to the old-35 promoter, an enzyme that specifically inhibits OLD-35 activity), or, alternatively, synthetic molecules such as (but not limited to) small molecules generated in a combinatorial chemical library. In sections 5.1.1 and 5.1.2, below, the assays identify agents that antagonize Old-35, but the skilled artisan would be able, given the instant disclosure, to use methods that look for an opposite result (e.g., an increase in promoter activity, an increase in reactive oxygen species, increased NF-κB binding or translocation into the nucleus, or increased cytokine levels) to identify agents that enhance rather than decrease the inflammatory activity of Old-35.

5.1.1 Assay Systems that Use the old-35 Promoter

The present invention provides for a method for identifying an agent that inhibits inflammation, comprising exposing a test agent to a system comprising an old-35 promoter element operatively linked to a reporter gene and determining whether the exposure to the test agent increases transcription of the reporter gene, wherein a decrease in transcription of the reporter gene indicates that the test agent inhibits inflammation.

As set forth above, the test agent may be a naturally occurring molecule or substance or may be synthetic.

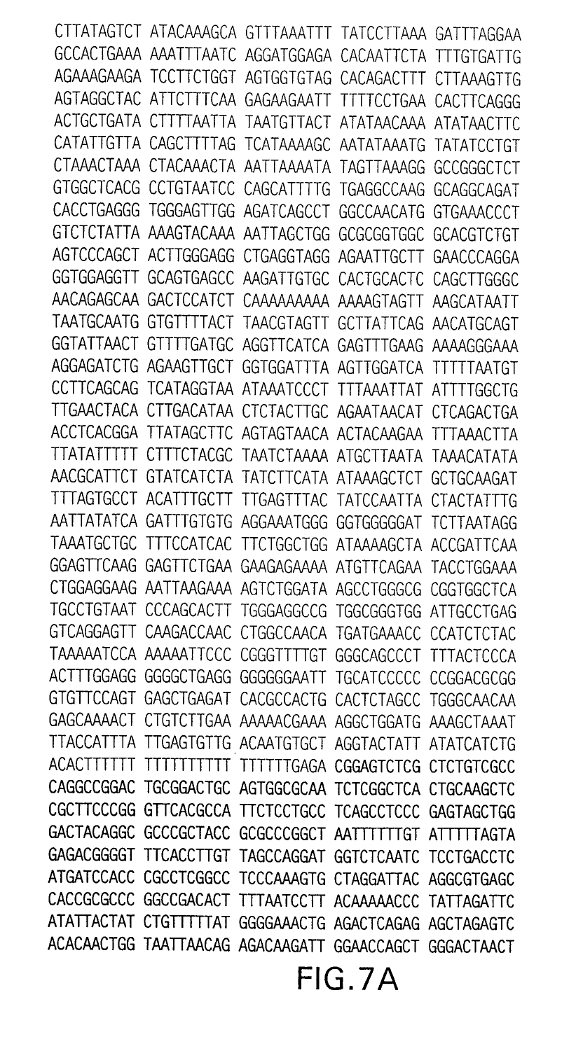

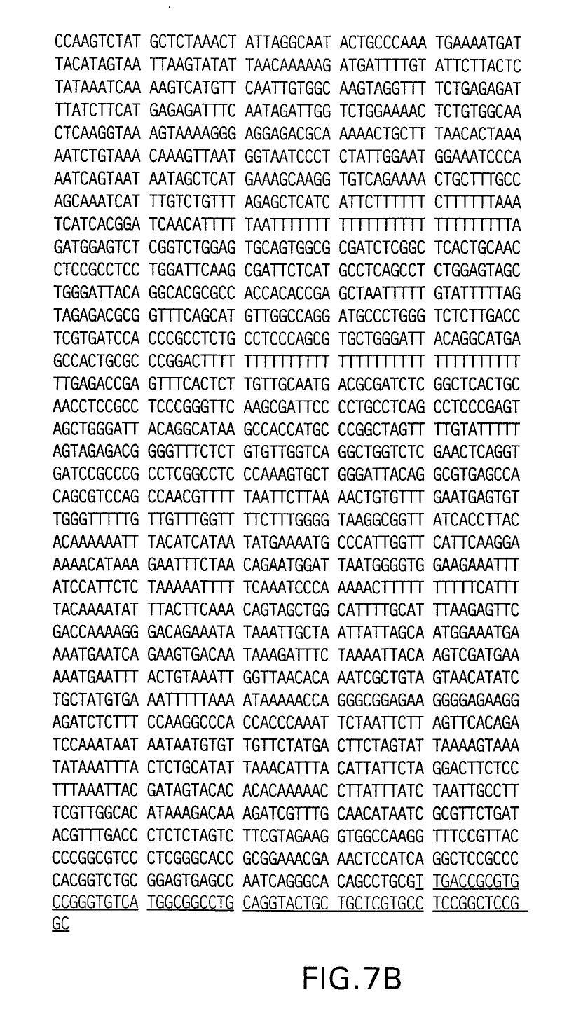

An old-35 promoter element is a promoter element that is found in nature operatively linked to an old-35 gene. The human old-35 promoter element has been cloned and characterized and is set forth in FIG. 7, as SEQ ID NO:1. In addition, the old-35 genes of mouse (GenBank Acc. No. AF465249) has been cloned and a person of ordinary skill in the art would be able to obtain the promoter element operatively linked to these non-human old-35 genes using standard laboratory techniques.

In preferred embodiments of the invention, the promoter element used is the human old-35 promoter (SEQ ID NO:1) or a variant thereof. The term “variant” includes fragments, deletion mutants, insertional mutants, point mutants, substitution mutants, nucleic acid molecules comprising one or more modified nucleic acid, etc. The wild-type old-35 promoter and variants thereof are collectively referred to as “old-35 promoters.” Preferably variants are at least 85 percent, preferably at least 90 percent homologous to a nucleic acid molecule having a sequence set forth in SEQ ID NO:1 (FIG. 7) and/or hybridize to a nucleic acid molecule having a sequence set forth in SEQ ID NO:1 (FIG. 7), or its complementary strand, under stringent conditions for detecting hybridization of nucleic acid molecules as set forth in “Current Protocols in Molecular Biology”, Volume I, Ausubel et al., eds. John Wiley: New York N.Y., pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68° C. is recommended, but the optimal temperature will depend on the length of the nucleic acid probe, its GC content, the concentration of monovalent cations and the percentage of formamide, if any, that was contained in the hybridization solution.

Deletion mutants of the old-35 promoter preferably hybridize to a nucleic acid molecule having a sequence as set forth in SEQ ID NO:1 under stringent conditions.

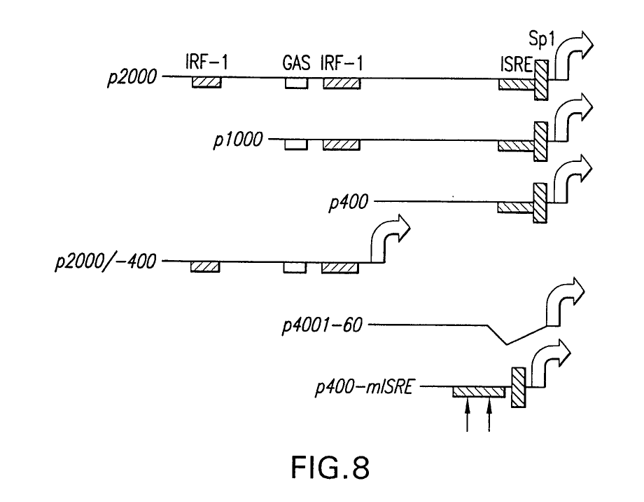

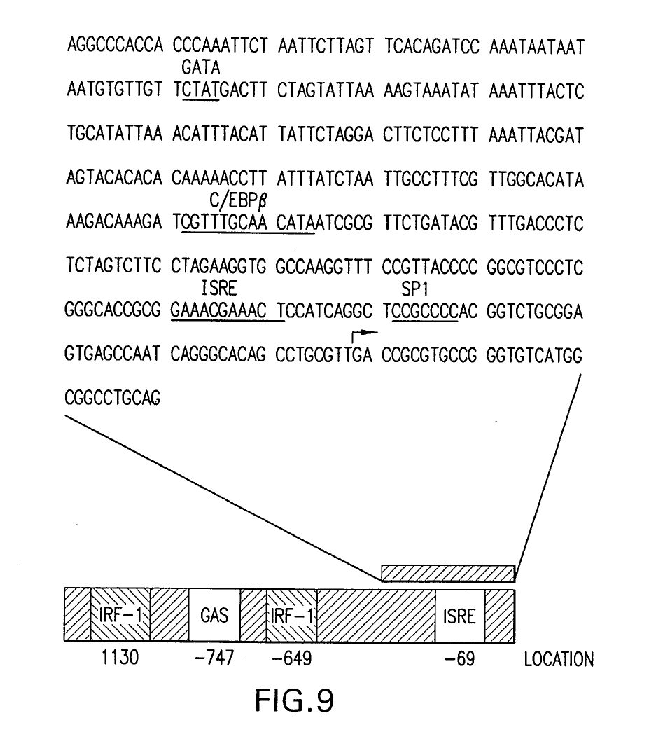

In one non-limiting embodiment of the invention, a human old-35 promoter variant is contained in p2000, as depicted in FIG. 8, and the sequence of which is set forth as bold face text in FIG. 7. Additional non-limiting examples of old-35 promoter variants include p1000, p400, p2000/-400, p400/-60 and p400-mISRE, also as depicted in FIG. 8 (with reference to FIG. 7 and SEQ ID NO:1). The nucleic acid sequence of p400 is depicted in FIG. 9 and SEQ ID NO:2.

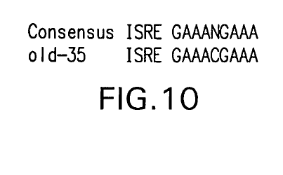

The present invention further provides for isolated nucleic acid molecules comprising subregions of an old-35 promoter, including but not limited to an old-35 Interferon-Stimulated Response Element (“ISRE”) having a sequence as set forth in SEQ ID NO:4 and depicted in FIG. 10.

Data demonstrating that IFN-β was more effective in upregulating p400 than the p2000 construct indicate that one or more repressor element(s) is present in the p2000 construct. It may be desirable to omit this one or more repressor element from constructs intended to optimize promoter activity. One non-limiting example of an old-35 promoter variant lacking the repressor is the p400 variant.

The present invention provides for an old-35 promoter operatively linked to a gene of interest which, when introduced into a suitable host cell, results in the transcription of the gene of interest and preferably in the expression of a protein encoded by the gene of interest. The gene of interest may be an old-35 gene or may be another gene (a “heterologous”) gene. Examples of non-old-35 genes of interest include but are not limited to reporter genes, such as the genes encoding green fluorescent protein (or another naturally occurring fluorescent protein or engineered variant thereof), β-glucuronidase, β-galactosidase, luciferase, and dihydrofolate reductase.

The transcriptional activity of the old-35 promoter/reporter gene construct may be evaluated in vitro (e.g., nuclear run-off assays) or in vivo. The construct may be introduced into a cell using standard techniques, including transformation, transduction, transfection, electroporation, etc. The construct, for propagation purposes or for introduction into a cell, may be incorporated into a suitable vector molecule, such as a plasmid, a phage, a phagemid or a virus. Where the vector is an expression vector, suitable expression vectors include virus-based vectors and non-virus based DNA or RNA delivery systems. Examples of appropriate virus-based gene transfer vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

A decrease in transcription of the reporter gene may be detected by measuring reporter gene mRNA, the protein product of the reporter gene, or a property of the reporter gene product (e.g., enzyme activity, fluorescence, etc.).

The decrease in transcription is preferably measured relative to a control in which the old-35 promoter/reporter gene construct is not exposed to test agent.

Preferably, the decrease in transcription is at least about 10, 30, 50, 70 or 90 percent.

5.1.2 Assay Systems that Use the Old-35 Gene

In a first set of non-limiting embodiments, the present invention provides for a method for identifying an agent that inhibits inflammation, comprising administering a test agent (that is a putative anti-inflammatory agent) to a cell comprising an old-35 gene operatively linked to a promoter element, wherein the old-35 gene is transcribed and expressed as OLD-35 protein, and determining whether the exposure to the test agent decreases the amount of reactive oxygen species in the cell. In preferred, non-limiting related embodiments of the invention, the cell is a test cell into which an old-35 gene operatively linked to a promoter element has been introduced, and the amount of reactive oxygen species in the test cell exposed to the test agent is decreased relative to the amount of reactive oxygen species in a first control cell into which the old-35 gene operatively linked to the promoter has been introduced, but which is not exposed to the test agent. Further, the amount of reactive oxygen species in the first control cell is greater than that in a second control cell which does not contain the old-35 gene operatively linked to the promoter element and which is not senescent or terminally differentiated.

Reactive oxygen species that may be measured include, but are not limited to, superoxide anions, hydroxyl radicals and hydrogen peroxide. Reactive oxygen species may be detected by methods known in the art, for example, but not by way of limitation, by staining with hydroethidine and dichlorofluorescein diacetate, followed by flow cytometry, as set forth in example section 6, below.

In a second set of non-limiting embodiments, the present invention provides for a method for identifying an agent that inhibits inflammation, comprising administering a test agent to a cell comprising an old-35 gene operatively linked to a promoter element, wherein the old-35 gene is transcribed and expressed as OLD-35 protein, and determining whether the exposure to the test agent decreases the amount of binding between NF-κB and its target sequence in the cell. In preferred non-limiting related embodiments, the cell is a test cell into which an old-35 gene operatively linked to a promoter element has been introduced, and the amount of binding between NF-κB and its target sequence in the test cell exposed to the test agent is decreased relative to the amount of binding between NF-κB and its target sequence in a first control cell into which the old-35 gene operatively linked to the promoter has been introduced, but which is not exposed to the test agent. Further; the amount of binding between NF-κB and its target sequence in the first control cell is greater than that in a second control cell which does not contain the old-35 gene operatively linked to the promoter element and which is not senescent or terminally differentiated.

Binding of NF-κB to its target sequence may be demonstrated by any method known in the art. For example, a cell, test cell or control cell may contain a transgene comprising a reporter gene, such as luciferase, operably linked to a promoter containing one or more NF-κB binding site, and the amount of reporter gene produced in the presence and absence of the test agent may be monitored. Alternatively, electrophoretic mobility shift assays may be performed using nuclear extracts. See, for example, section 6 below.

In a third set of non-limiting embodiments, the present invention provides for a method for identifying an agent that inhibits inflammation, comprising administering a test agent to a cell comprising an old-35 gene operatively linked to a promoter element, wherein the old-35 gene is transcribed and expressed as OLD-35 protein, and determining whether the exposure to the test agent decreases the amount of translocation of a NF-κB protein from the cytoplasm into the nucleus of the cell. In preferred non-limiting related embodiments, the cell is a test cell into which an old-35 gene operatively linked to a promoter element has been introduced, and the amount of translocation of a NF-κB protein from the cytoplasm into the nucleus in the test cell exposed to the test agent is decreased relative to the amount of translocation of a NF-κB protein from the cytoplasm into the nucleus in a first control cell into which the old-35 gene operatively linked to the promoter has been introduced, but which is not exposed to the test agent. Further, the amount of translocation of a NF-κB protein from the cytoplasm into the nucleus in the first control cell is greater than that in a second control cell which does not contain the old-35 gene operatively linked to the promoter element and which is not senescent or terminally differentiated.

The translocation of a NF-κB protein from the cytoplasm into the nucleus may be detected and measured using standard laboratory techniques, for example, by cell fractionation into nuclear and cytoplasmic fractions followed by Western blot analysis using NF-κB specific antibodies (see example section 6, below).

In a fourth set of non-limiting embodiments, the present invention provides for a method for identifying an agent that inhibits inflammation, comprising administering a test agent to a cell comprising an old-35 gene operatively linked to a promoter element, wherein the old-35 gene is transcribed and expressed as OLD-35 protein, and determining whether the exposure to the test agent decreases the amount of a cytokine selected from the group consisting of interleukin-6, interleukin-8, TNFR1, RANTES and MMP-3 in the cell. In preferred related, non-limiting embodiments, the cell is a test cell into which an old-35 gene operatively linked to a promoter element has been introduced, and the amount of a cytokine selected from the group consisting of interleukin-6, interleukin-8, TNFR1, RANTES and MMP-3 in the test cell exposed to the test agent is decreased relative to the amount of a cytokine selected from the group consisting of interleukin-6, interleukin-8, TNFR1, RANTES and MMP-3 in a first control cell into which the old-35 gene operatively linked to the promoter has been introduced, but which is not exposed to the test agent. Further; the amount of a cytokine selected from the group consisting of interleukin-6, interleukin-8, TNFR1, RANTES and MMP-3 in the first control cell is greater than that in a second control cell which does not contain the old-35 gene operatively linked to the promoter element and which is not senescent or terminally differentiated. Cytokines may be measured using methods known in the art, including but not limited to ELISA analysis, Western blot, or antibody array using anti-cytokine antibodies (see example section 6, below) or Northern analysis of cellular RNA using cytokine specific nucleic acid probes.

In the foregoing four sets of embodiments, where a promoter/old-35 construct is not introduced, the cell may naturally express the old-35 gene, and may be identified as expressing the old-35 gene at a level that is at least equal to the level expressed in neonatal human fibroblasts. Examples of such cells include senescent cells, including cells of progeria patients, and terminally differentiated cells, such as melanoma cells exposed to interferon beta and mezerein (see United States Patent Application Publication No. 20030099660). The cell or test cell may be any cell that permits expression of OLD-35. Where a promoter/old-35 construct is introduced into the test cell, the test cell absent the construct preferably (but not necessarily) produces undetectable or low levels of OLD-35, to minimize background.

Any promoter that is active or inducible in the cell, test cell and/or first control cell may be used. Non-limiting examples of such promoters include the cytomegalovirus immediate early promoter, the Rous sarcoma virus long terminal repeat promoter, the human elongation factor lea promoter, the human ubiquitin c promoter, etc. Non-limiting examples of inducible promoters include the murine mammary tumor virus promoter (inducible with dexamethasone); commercially available tetracycline-responsive or ecdysone-inducible promoters, etc.

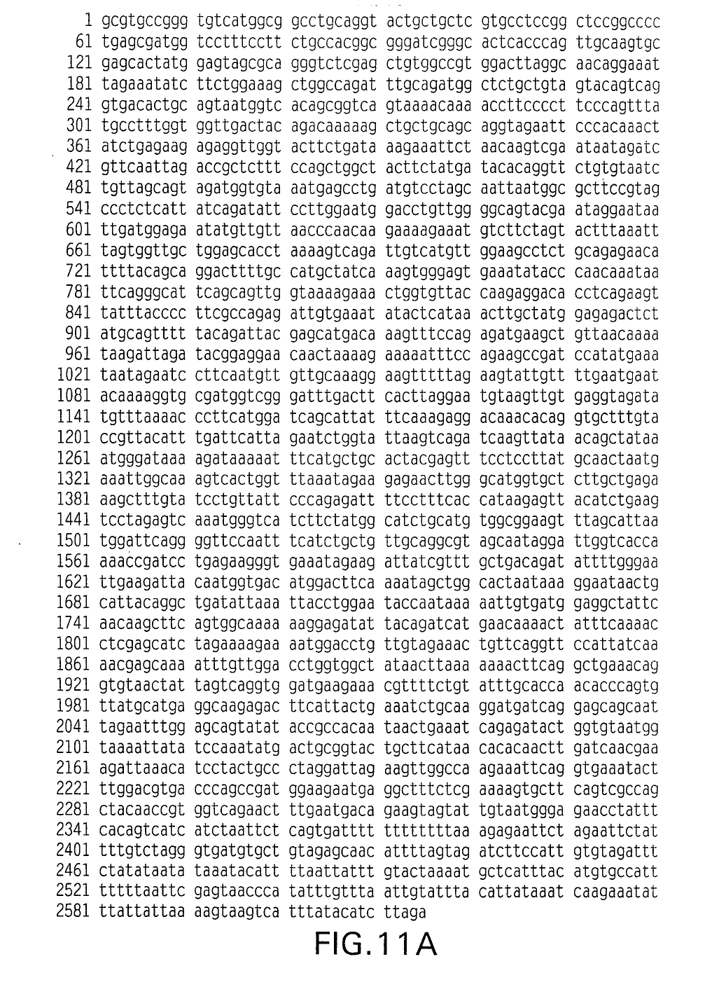

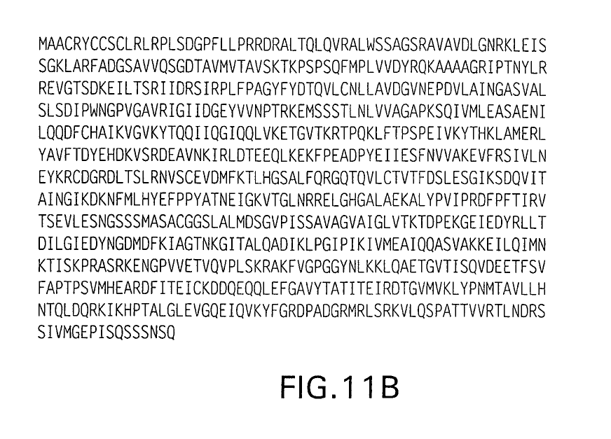

Old-35 genes which may be used according to the invention include the human old-35 gene, having a nucleic acid sequence as deposited in GenBank Accession No. AY027528 SEQ ID NO:5, or a nucleic acid that hybridizes thereto under stringent conditions (see section 5.1.1, above). In addition, a nucleic acid comprising an old-35 gene may encode human OLD-35 protein having a sequence as set forth in FIG. 11, GenBank Accession No. AY027528 and SEQ ID NO: 6. The present invention further comprises OLD-35 fusion proteins and nucleic acids encoding the same.

In addition, non-human forms of old-35 may also be used according to the invention. For example, where murine assay systems are used, murine old-35 homolog, GenBank Acc. No. AF465249, may optionally be used.

The promoter/old-35 construct may be introduced into a cell using standard techniques, including transformation, transfection, transduction, electroporation, etc. For propagation or for introduction into a host cell, the construct may be comprised in a vector molecule, such as a plasmid, phage, phagemid, or virus (see section 5.1.1, above). In a preferred embodiment, the construct may be comprised in a recombinant adenovirus vector wherein transcription of the old-35 cDNA is driven by the cytomegalovirus immediate early (CMV) promoter.

5.2 Model Systems of Inflammation

The present invention provides for cells and animals engineered to overexpress (in the case of animals, in at least some cells and tissues) an old-35 gene, and thereby serve as model systems for the study of inflammation and for the evaluation of agents that modify inflammation (for example, for the discovery of novel anti-inflammatory compounds).

Suitable transgenic animals include, but are not limited to, mice, rats, goats, sheep, pigs, cows, etc.

Old-35 may be over expressed in some or all cells of said animals. Overexpression may be limited to certain tissues to provide model systems of particular inflammatory conditions. A number of non-limiting examples of such systems follow.

In one set of non-limiting embodiments, the present invention provides for a model system of arthritis, comprising a non-human animal carrying a transgene comprising an old-35 gene operatively linked to a promoter element that is selectively active in cells comprised in a joint of the animal. Examples of such promoters include but are not limited to the chondromodulin-1 (ChM-I) gene promoter (Aoyama et al., J Biol Chem. 2004, 279:28789-28797), chicken collagen X regulatory sequences (Campbell et al., Am J Pathol. 2004, 164:487-499, connective tissue growth factor (CTGF/Hcs24) gene promoter (Kubota et al., Bone. 2003; 33:694-702), the 4-kb murine Col10a1 promoter of the alpha1 (X) collagen gene (Zheng et al., J. Cell Biol. 2003; 162:833-842), cartilage oligomeric matrix protein gene (COMP) promoter (Issack et al., J Orthop Res. 2004, 22:751-758).

In another set of non-limiting embodiments, the present invention provides for a model system for atherosclerosis, comprising a non-human animal carrying a transgene comprising an old-35 gene operatively linked to a promoter element that is selectively active in cells of the vascular system. Examples of such promoters include but are not limited to the Angiopoietin-2 (Ang-2) gene promoter (Hegen et al., Arterioscler Thromb Vasc Biol. 2004 Jul. 29 [Epub ahead of print]), endothelial nitric-oxide synthase (eNOS) gene promoter (Chan et al., J Biol Chem. 2004, 279:35087-35100), Cysteine-rich protein (CRP)2 gene promoter (Chang et al., Am J Physiol Heart Circ Physiol. 2003 285:H1675-1683). intercellular adhesion molecule 2 (ICAM-2), platelet endothelial cell adhesion molecule 1 (PECAM-1) and endoglin. gene promoters (Cowan et al., Xenotransplantation. 2003; 10:223-231).

In yet another set of non-limiting embodiments, the present invention provides for a model system for Alzheimer's disease, comprising a non-human animal carrying a transgene comprising an old-35 gene operatively linked to a promoter element that is selectively active in cells of the central nervous system. Examples of such promoters include the neuron-specific enolase gene promoter (Tanaka et al., 2001, Anticancer Res. 21:291-294) and the excitatory amino acid transporter-2 promoter, as set forth in GenBank Acc. No. AF510107.

To study or monitor inflammation in said cells or animals, the present invention provides for a method for evaluating inflammation in a transgenic non-human animal carrying a transgene comprising an old-35 gene operatively linked to a promoter element, comprising determining, in a cell, tissue, or fluid of the animal, whether the amount of reactive oxygen species is increased, whether the amount of binding of a NF-κB protein to its target sequence is increased, whether the amount of a NF-κB protein translocated into the nucleus is increased, or whether the amount of a cytokine selected from the group consisting of interleukin-6, interleukin-8, TNFR1, RANTES and MMP-3 is increased. Such determinations may be made using methods known in the art (see section 5.1.2 above and example section 6 below).

The present invention also provides for administering a test agent to a transgenic animal carrying a transgene comprising an old-35 gene, and then determining whether inflammation is increased or decreased in said animal. Such transgenic animals may be used to discover new anti-inflammatory agents or to demonstrate anti-inflammatory activity of a putative or known anti-inflammatory agent.

Such transgenic animals may provide the advantage of creating a model system of inflammation as it occurs with aging.

5.3 Diagnostic Methods and Kits

The present invention provides for a method of detecting inflammation in a subject, comprising determining whether there is an increase in the expression of an old-35 gene in a cell of the subject relative to a control cell. Such an increase may be determined by showing an increase in old-35 mRNA (for example by Northern blot or dot-blot analysis) using a suitable old-35 nucleic acid probe, or by showing an increase in OLD-35 protein using, for example, an antibody directed toward OLD-35. The method may be practiced in vivo or preferably in vitro.

Inflammation may be detected so as to demonstrate acute or chronic inflammation. Detection of increased expression of Old-35 (nucleic acid and/or protein) may support a diagnosis of a chronic inflammatory condition such as, but not limited to, arthritis, atherosclerosis, periodontal disease, Alzheimer's disease or chronic obstructive pulmonary disease.

In related embodiments, the present invention provides for a kit for detecting inflammation in a subject, comprising a probe that binds to an old-35 gene product selected from the group consisting of old-35 mRNA (in which case the probe is a nucleic acid) and OLD-35 protein (in which case the probe is an antibody) and an antibody that binds to a cytokine selected from the group consisting of interleukin-6, interleukin-8, TNFR1, RANTES and MMP-3. Alternatively, the kit may comprise a nucleic acid probe that may be used to detect cytokine mRNA.

5.4 Methods of Treatment and Anti-Inflammatory Compositions

The present invention provides for a method of inhibiting inflammation in a subject in need of such treatment, comprising administering, to the subject, an effective amount of an agent that antagonizes the expression and/or activity of OLD-35, including, but not limited to, an effective amount of an antibody that binds to OLD-35 protein, an old-35 RNA-I, or an antisense old-35 nucleic acid.

As one non-limiting example, one of the foregoing Old-35 antagonist agents may be administered into the synovial fluid of an inflamed joint.

In non-limiting embodiments, the amount of antagonist may be administered to achieve a concentration of, for an OLD-35 antibody (10 μg-1 mg/ml), for an old-35 RNA-i or antisense RNA (25-100 nM).

The present invention further provides for a method of inhibiting inflammation in a subject in need of such treatment, comprising introducing, into cells of the subject, a nucleic acid comprising an old-35 promoter element operatively linked to a gene that inhibits inflammation. Non-limiting examples of such genes include antisense NFkB p65 subunit, dominant negative versions of IkB or NFkB p65 subunit etc.

The present invention still further provides for an anti-inflammatory composition, comprising an agent that antagonizes old-35 activity selected from the group consisting of an old-35 RNA-i, an old-35 antisense RNA, and an antibody directed toward OLD-35, and a second anti-inflammatory agent. Non-limiting examples of second anti-inflammatory agents include a steroid compound or a non-steroidal anti-inflammatory agent such as aspirin, ibuprofen, naprosyn, celecoxib, valdecoxib, diclofenac, and anti-inflammatory antibodies (or their fragments) such as etanercept, infliximab or anakinra.

5.5 Active Subregions of OLD-35

5.5.1 Domains of OLD-35

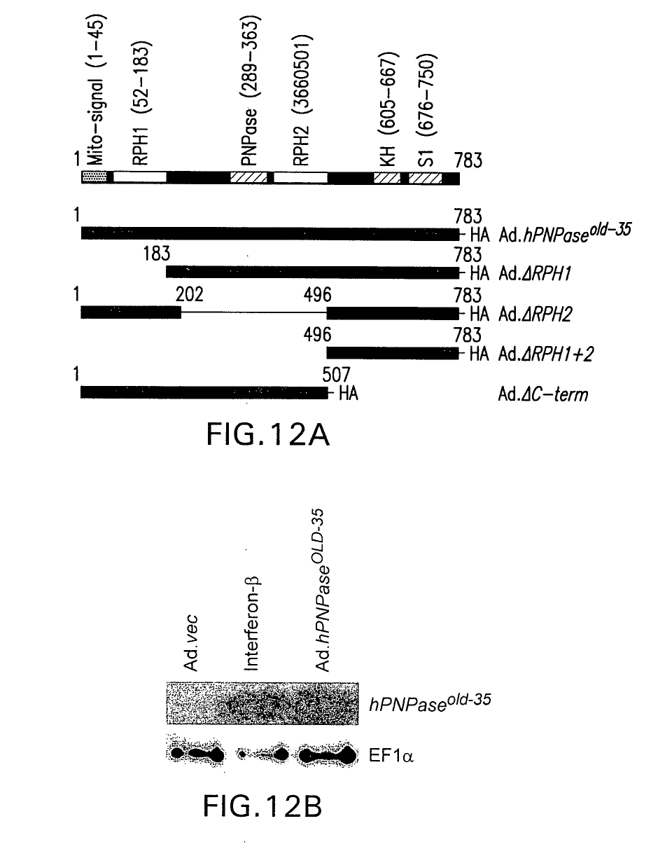

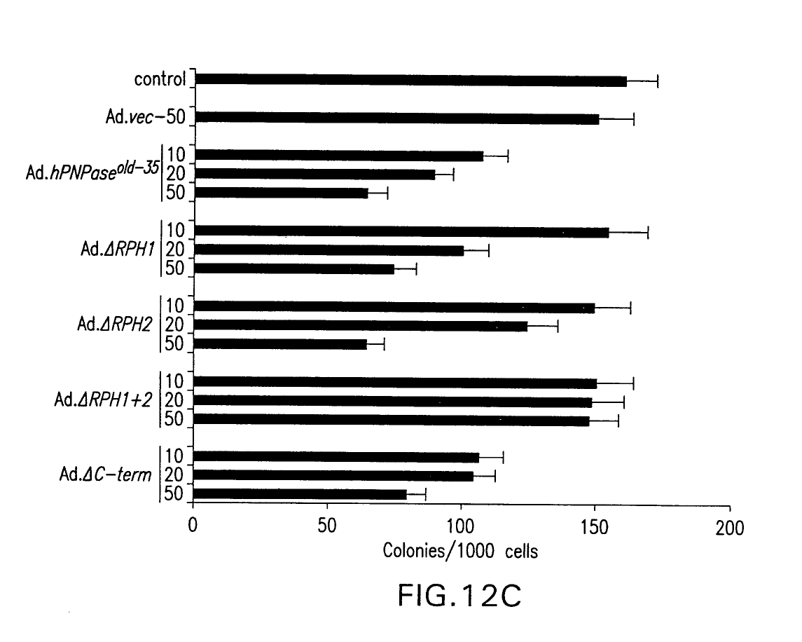

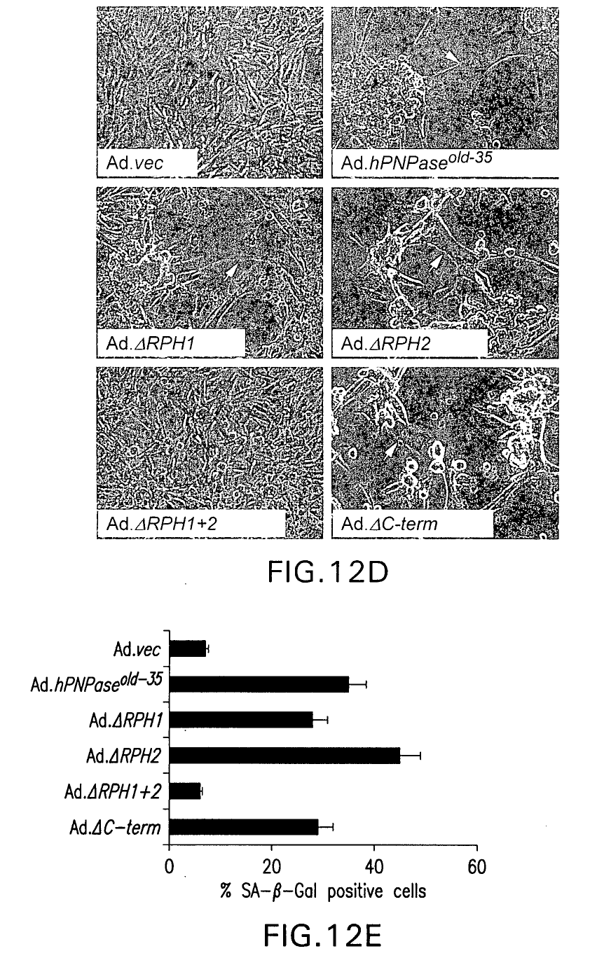

The present invention provides for a group of proteins collectively referred to as “OLD-35 variants” comprising active subregions of the OLD-35 protein. “Active” refers to anti-proliferative activity, inflammatory activity, PNPase activity, RNA degradation activity, cell-cycle arresting/slowing activity, and/or senescence inducing activity. Old-35 belongs to an RNA processing enzyme family comprising the polynucleotide phosphorylases (PNPases). PNPases typically contain RNAse PH (RPH) domains and RNA binding domains. Native full length OLD-35 protein contains 783 amino acid residues (SEQ ID NO:6, FIG. 11B) comprising the following domains: (i) a mitochondrial localization signal at amino acids 1-45 (SEQ ID NO:20, FIG. 12); (ii) two RPH domains, involved in RNA degradation, one at amino acids 52-183 (SEQ ID NO:15, FIG. 12), the other at amino acids 366-501 (SEQ ID NO:16, FIG. 12); (iii) an α-helix PNPase domain at amino acids 289-363 (SEQ ID NO:21, FIG. 12) involved in RNA binding; (iv) a RNA binding KR domain at amino acids 605-667 (SEQ ID NO:22, FIG. 12); and (v) a RNA binding S1 domain at amino acids 676-750 (SEQ ID NO:23, FIG. 12).

In one embodiment of the invention an OLD-35 variant is a protein that is not the amino acid sequence set forth as SEQ ID NO:6, which encodes the full length OLD-35 protein consisting of 783 amino acids, but that comprises a RPH domain as set forth in SEQ ID NOS: 15-19 (FIG. 12), or that is at least about 85, at least about 90, or at least about 95 percent homologous thereto (where “homology” as that term is used herein, may be determined using standard homology search software such as BLAST or FASTA). The invention also provides for a nucleic acid encoding an OLD-35 variant protein as set forth in SEQ ID NOS:15-19, comprising coding regions in the sequence set forth in SEQ ID NOS:24-28, or another sequence which, when translated, produces a protein having essentially the same amino acid sequence as SEQ ID NOS: 15-19. Preferably nucleic acids encoding Old-35 variants are at least 85 percent, preferably at least 90 to 95 and most preferably 100 percent homologous to the nucleic acid molecules having a sequence set forth in SEQ ID NOS:24-28 and/or hybridize to a nucleic acid molecule having a sequence set forth in SEQ ID NOS:24-28, or its complementary strand, under stringent conditions for detecting hybridization of nucleic acid molecules as set forth in “Current Protocols in Molecular Biology”, Volume I, Ausubel et al., eds. John Wiley: New York N.Y., pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68° C. is recommended, but the optimal temperature will depend on the length of the nucleic acid probe, its GC content, the concentration of monovalent cations and the percentage of formamide, if any, that was contained in the hybridization solution.

As set forth in SEQ ID NOS: 17-19, an Old-35 variant may comprise of either one or both RPH domains and any additional domain such as the mitochondrial localization signal (SEQ ID NO:20), the PNPase domain (SEQ ID NO:21), the KH domain (SEQ ID NO:23) or the S1 domain (SEQ ID NO:24) or that is at least about 85, at least about 90, or at least about 95 percent homologous thereto. In an additional non-limiting embodiment, an Old-35 variant need not have a contiguous arrangement of various its domains in the same linear order as present in the native OLD-35 protein.

In particular, non-limiting embodiments, the present invention provides for an OLD-35 variant protein comprising one, but not two, RPH domain and having an activity selected from the group consisting of anti-proliferative activity, PNPase activity, RNA degradation activity, cell-cycle arrest/slowing activity, senescence-inducing activity and a combination thereof. In an alternative non-limiting embodiment, an OLD-35 variant protein may comprise one, but not two, RPH domain which is immunogenic in a mammal. The invention also provides for an OLD-35 variant possessing anti-proliferative activity, PNPase activity, RNA degradation activity, cell-cycle slowing activity, senescence-inducing activity, a combination thereof and which is immunogenic in mammals, comprising amino acid residues 52-183 (SEQUENCE ID NO:15) or amino acid residues 366-501 (SEQUENCE ID NO:16) of native OLD-35 protein, or a sequence that is at least 90 percent, preferably at least 95 percent homologous to residues 52-183 or residues 366-501 respectively. The invention also provides for an OLD-35 variant possessing anti-proliferative activity, PNPase activity, RNA degradation activity, cell-cycle slowing activity, senescence-inducing activity and a combination thereof and which is immunogenic in mammals, comprising amino acid residues 289-363 (SEQUENCE ID NO:21) of native OLD-35 protein, or a sequence that is at least 90 percent, preferably at least 95 percent homologous to residues 289-363.

Accordingly, in specific, nonlimiting embodiments, the present invention provides for Old-35 variants as follows, where the residues identified are as set forth in SEQ ID NOS:

(i) lacking residues 676-750, and comprising residues 52-183 and residues 299-363, operably joined, optionally by a linker sequence, and having PNPase activity;

(ii) lacking residues 676-750, comprising residues 366-501 and residues 289-363, operably joined, optionally by a linker sequence, and having PNPase activity;

(iii) lacking residues 1-45, and comprising residues 52-183 and having anti-proliferative activity;

(iv) lacking residues 1-45, and comprising residues 366-501, and having antiproliferative activity;

(v) not the complete native OLD-35 sequence, comprising residues 289-363, and immunogenic in a mammal;

(vi) not the complete native OLD-35 sequence, comprising residues 366-501, and immunogenic in a mammal.

(vii) not the complete native OLD-35 sequence, comprising residues 607-667, and immunogenic in a mammal;

(viii) not the complete native OLD-35 sequence, comprising residues 676-750, and immunogenic in a mammal;

5.5.2 Expression Systems

The Old-35 variants of the invention may be produced by any method known in the art. Such methods include but are not limited to chemical synthesis and recombinant DNA techniques.

With regard to production of Old-35 variants using recombinant DNA techniques, the present invention provides for nucleic acids encoding said variants. Such nucleic acids may either be nucleic acid fragments of the aforelisted Old-35 nucleic acids derived from SEQ ID NO:5- and include SEQ ID NOS:24-28, encoding the variants, or may be nucleic acids designed, using the genetic code, to encode such variants, wherein an alternate or optimized codon usage provides the basis for conservative codon or amino acid substitutions.

A nucleic acid encoding a Old-35 variant of the invention may be comprised in a suitable vector molecule, and may optionally be operatively linked to a suitable promoter element, for example, but not limited to, the cytomegalovirus immediate early promoter, the Rous sarcoma virus long terminal repeat promoter, the human elongation factor 1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In specific non-limiting embodiments of the invention, the promoter may be selectively active in cancer cells; one example of such a promoter is the PEG-3 promoter, as described in International Patent Application No. PCT/US99/07199, Publication No. WO 99/49898 by Fisher et al., published on Oct. 7, 1999; other non-limiting examples include the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takakuwa et al., 1997, Jpn. J. Cancer Res. 88:166-175), the human carcinoembryonic antigen gene promoter (Lan et al., 1996,Gastroenterol. 111:1241-1251), the gastrin-releasing peptide gene promoter (Inase et al., 2000, Int. J. Cancer 85:716-719). the human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the midkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (Genbank Accession No. AF241535), which is particularly active in pancreatic cancer cells (Perrais et al., J Biol Chem. 2001, 276:30923-30933).

Suitable expression vectors include virus-based vectors and non-virus based DNA or RNA delivery systems. Examples of appropriate virus-based gene transfer vectors include, but are not limited to, pCEP4 and pREP4 vectors from Invitrogen, and, more generally, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6:113-138; Connelly, 1999, Curr. Opin. Mol. Ther. 1:565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996,Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 198:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

Old-35 variant protein may also be produced by yeast or bacterial expression systems. For example, bacterial expression may be achieved using plasmids such as pGEX expression system (Amersham Biosciences, Piscataway, N.J.), pQE His-tagged expression system (Qiagen, Valencia, Calif.), pET His-tagged expression system (EMD Biosciences, Inc., La Jolla, Calif.), or IMPACT expression system (New England Biolabs, Beverly, Mass.).

In a specific, non-limiting embodiment of the invention, a nucleic acid encoding an Old-35 variant, in expressible form, may be in vitro translated to produce OLD-35 variant protein. In vitro translation may be performed using an appropriate system such as the TNT® coupled Reticulocyte Lysate Systems (Promega) or TNT® Coupled Wheat Germ Extract Systems. The expressed protein may include an affinity tag so that an Old-35 variant protein may be purified and recovered from the extracts for further use.

5.5.3 Delivery Systems

Depending on the expression system used, nucleic acid may be introduced by any standard technique, including transfection, transduction, electroporation, bioballistics, microinjection, etc.

In preferred, non-limiting embodiments of the invention, the expression vector is an E1-deleted human adenovirus vector of serotype 5. To prepare such a vector, an expression cassette comprising a transcriptional promoter element operatively linked to a Old-35 variant coding region and a polyadenylation signal sequence may be inserted into the multiple cloning region of an adenovirus vector shuttle plasmid, for example pXCJL.1 (Berkner, 1988, Biotechniques 6:616-624). In the context of this plasmid, the expression cassette may be inserted into the DNA sequence homologous to the 5′ end of the genome of the human serotype 5 adenovirus, disrupting the adenovirus E1 gene region. Transfection of this shuttle plasmid into the E1-transcomplementing 293 cell line (Graham et al., 1977, J. General Virology 36:59-74), or another suitable cell line known in the art, in combination with either an adenovirus vector helper plasmid such as pJM17 (Berkner, 1988, Biotechniques 6:616-624; McGrory et al., 1988, Virology 163:614-617) or pBHG10 (Bett et al., 1994, Proc. Natl. Acad. Sci. U.S.A. 91: 8802-8806) or a ClaI-digested fragment isolated from the adenovirus 5 genome (Berkner, 1988, Biotechniques 6:616-624), allows recombination to occur between homologous adenovirus sequences contained in the adenovirus shuttle plasmid and either the helper plasmid or the adenovirus genomic fragment. This recombination event gives rise to a recombinant adenovirus genome in which the cassette for the expression of the foreign gene has been inserted in place of a functional E1 gene. When transcomplemented by the protein products of the human adenovirus type 5 E1 gene (for example, as expressed in 293 cells), these recombinant adenovirus vector genomes can replicate and be packaged into fully-infectious adenovirus particles. The recombinant vector can then be isolated from contaminating virus particles by one or more rounds of plaque purification (Berkner, 1988, Biotechniques 6:616-624), and the vector can be further purified and concentrated by density ultracentrifugation.

In a specific, non-limiting embodiment of the invention, a nucleic acid encoding an Old-35 variant, in expressible form, may be inserted into the modified Ad expression vector pAd.CMV (Falck-Pedersen et al., 1994, Mol. Pharmacol. 45:684-689).

This vector contains, in order, the first 355 base pairs from the left end of the adenoviris genome, the cytomegalovirus immediate early promoter, DNA encoding splice donor and acceptor sites, a cloning site for the Old-35 variant gene, DNA encoding a polyadenylation signal sequence from the globin gene, and approximately three kilobase pairs of adenovirus sequence extending from within the EIB coding region. This construct may then be introduced into 293 cells (Graham et al., 1977, J. Gen. Virol. 36:59-72) together with plasmid JM17 (above), such that, as explained above, homologous recombination can generate a replication defective adenovirus containing Old-35 variant encoding nucleic acid.

5.6 Use of OLD-35 Variants

5.6.1 Use of OLD-35 Variants as Antiproliferative Agents

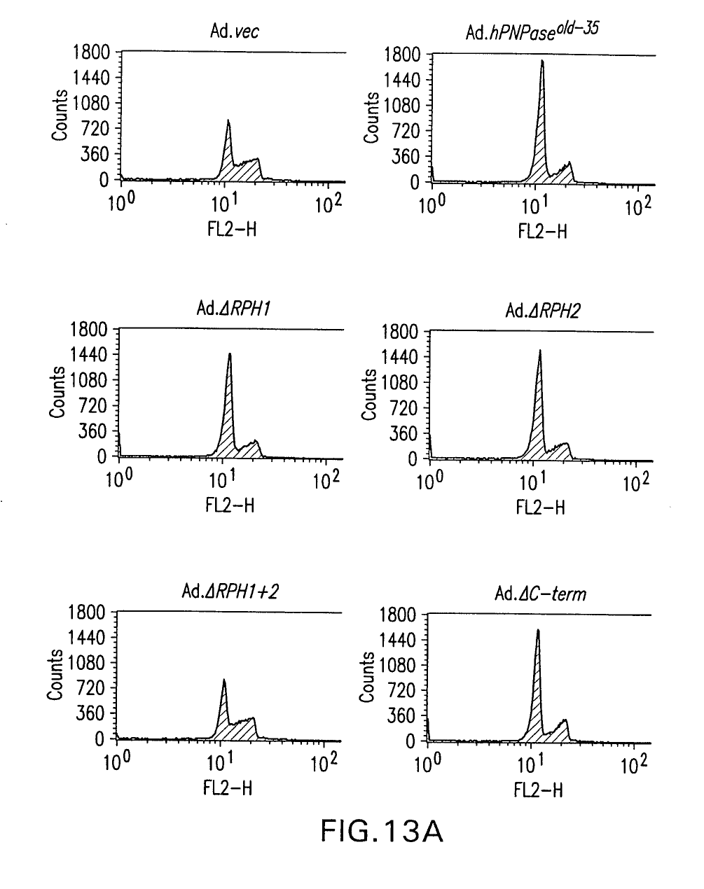

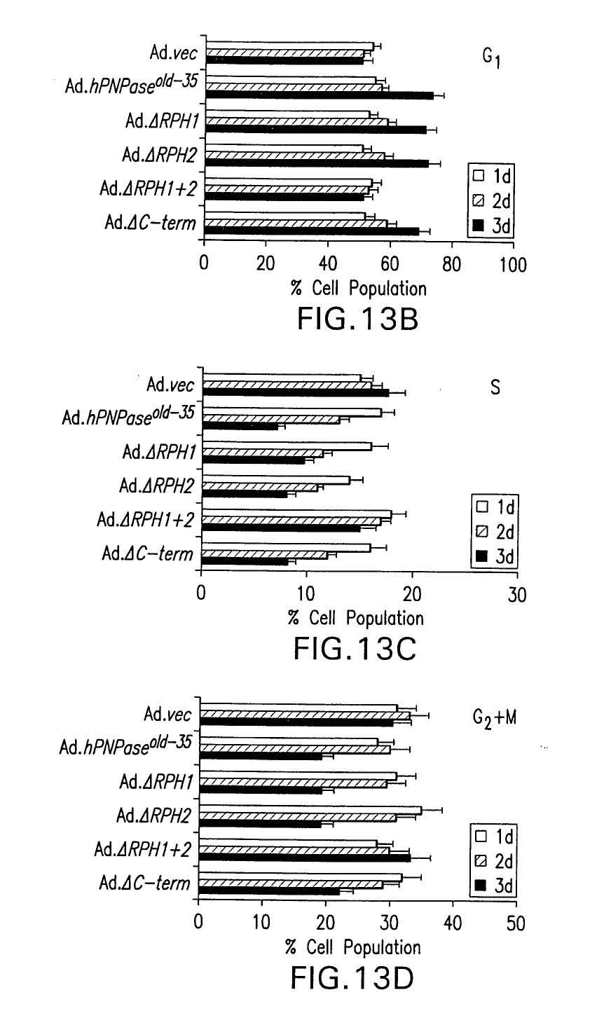

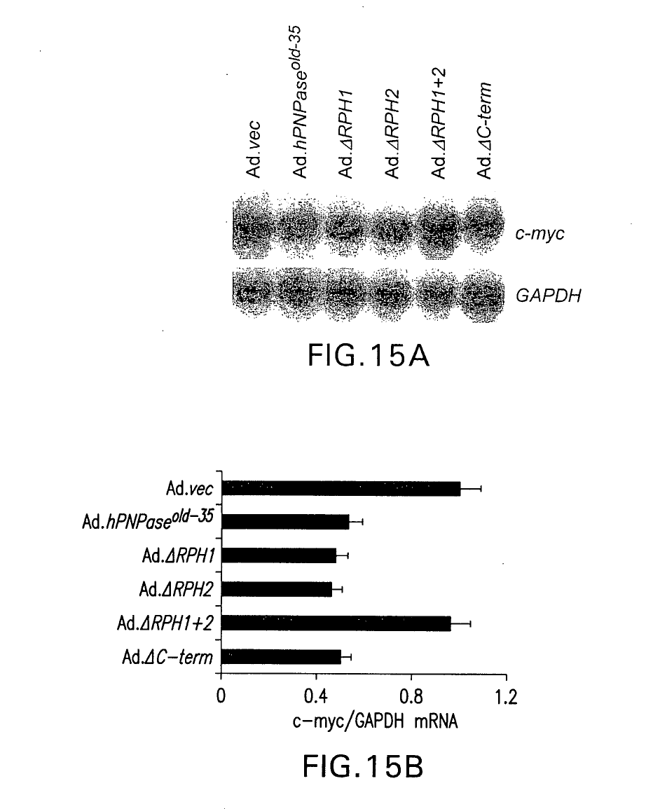

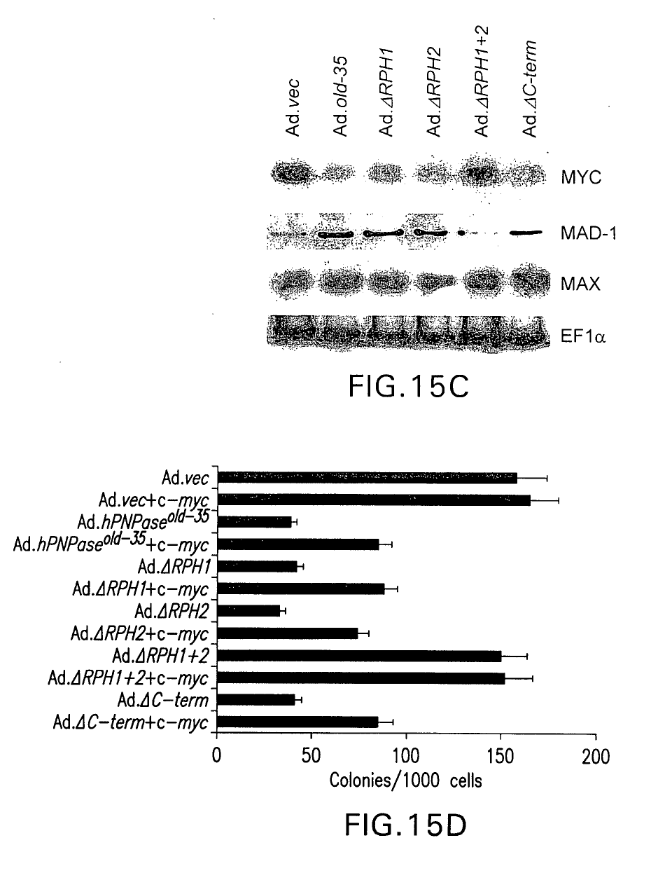

Old-35 variants are as effective as full length OLD-35 protein in inducing senescence or differentiation (FIG. 12), inhibiting colony formation (FIG. 12), causing cell cycle arrest (FIG. 13) and inhibiting the expression of c-myc protein by degrading c-myc mRNA (FIG. 15-17). In specific non-limiting embodiments an OLD-35 variant may be used to modulate cell proliferation in a subject, wherein a nucleic acid encoding the variant, in expressible form, may be introduced into a cell of the subject.

In preferred, non-limiting embodiments, the nucleic acid encoding the OLD-35 variant may be contained in a viral vector, operably linked to a promoter element that is inducible or constitutively active in the target cell. In preferred, non-limiting embodiments, the viral vector is a replication-defective adenovirus (as described in section 5.5.3 above).

In a specific, non-limiting embodiment of the invention, a viral vector containing a nucleic acid encoding a OLD-35 variant, such as a single RPH or two RPH domain containing proteins operably linked to a suitable promoter element, may be administered to a population of target cells at a multiplicity of infection (MOI) ranging from 10-100 MOI.

In another specific, non-limiting embodiment, the amount of a viral vector administered to a subject may be 1×10⁹ pfu to 1×10¹² pfu.

In specific, non-limiting embodiments, a nucleic acid encoding an OLD-35 variant comprised in a vector or otherwise, may be introduced into a cell ex vivo and then the cell may be introduced into a subject. For example, a nucleic acid encoding a OLD-35 variant may be introduced into a cell of a subject (for example; an irradiated tumor cell, glial cell or fibroblast) ex vivo and then the cell containing the nucleic acid may be optionally propagated and then (with its progeny) introduced into the subject.

5.6.2 Use of OLD-35 Variants as an Anti-Inflammatory Vaccine

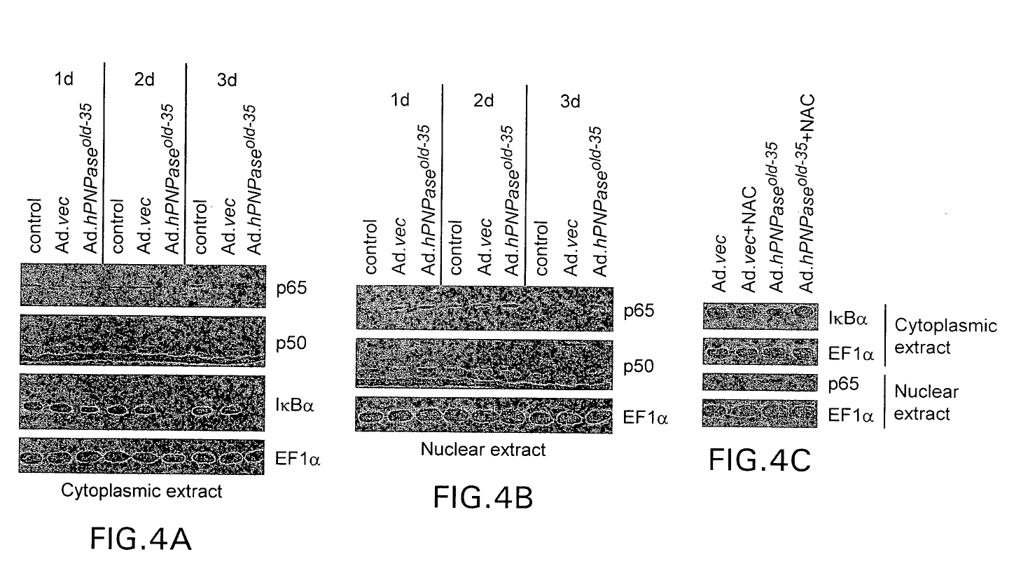

The present invention also provides for administering an OLD-35 variant in protein or peptide form in a subject in need of treatment for an acute or chronic inflammatory condition. Enhanced levels and activity of full length OLD-35 protein is associated with enhancing the levels of reactive oxygen species, inducing activation of the NF-κB pathway and enhancing the level of proinflammatory cytokines (FIGS. 4-6). Thus, in a specific non-limiting embodiment the present invention provides for delivery of an OLD-35 variant peptide to a subject so as to elicit an immune reaction against the injected peptide. Antibodies raised in a subject against an OLD-35 variant will also be active in binding to an endogenously expressed wild type OLD-35 protein. Thus, injection of an OLD-35 variant will act as a vaccine against an inflammatory response by reducing effective amounts of inflammation causing OLD-35 protein in a subject.

As such, the OLD-35 variant of the invention may be prepared by chemical synthesis or recombinant DNA techniques, purified by methods known in the art, and then administered to a subject in need of such treatment. An OLD-35 variant may be comprised, for example, in solution, in suspension, and/or in a carrier particle such as microparticles, liposomes, or other protein-stabilizing formulations known in the art In a non-limiting specific example, formulations of OLD-35 variant peptides may stabilized by addition of zinc and/or protamine stabilizers as in the case of certain types of insulin formulations. Alternatively, in specific non-limiting embodiments, an OLD-35 variant may be linked covalently or non-covalently, to a carrier protein which is preferably non-immunogenic.

In preferred, non-limiting embodiments, an OLD-35 variant protein/peptide is administered in an amount which achieves a local concentration in the range of 18 to 50 ng per microliter. For example, a subject may be administered a range of 50-100 mg per kilogram. For a human subject, the dose range may be between 1000-2500 mg/day.

### 6. EXAMPLE

### OLD-35 is an Agent of Inflammation

6.1 Materials and Methods

Cell Lines, Reagents and Virus Infection protocol. The human cervical carcinoma cell line HeLa was cultured in Dulbecco's Modified Eagle Medium (DMEM) supplemented with 10% fetal calf serum, penicillin (50 units/ml) and streptomycin (50 μg/ml) at 37° C. in 5% CO₂ and 100% relative humidity. N-acetyl-L-cysteine and Tiron were obtained from Sigma (St. Louis, Mo.). The recombinant replication-incompetent adenovirus expressing old-35 (Ad.hPNPase) was created in two steps as described previously and plaque purified by standard procedures (1, 28). Cells were infected with a multiplicity of infection (m.o.i.) of 1 to 50 plaque forming units (pfu)/cell of Ad.vec (control replication-incompetent adenovirus) or Ad.hPNPase as described (29).

Transient Transfection and Luciferase Assay. Cells (5×10³/well in 12-well plates) were either uninfected or infected with either Ad.vec or Ad. hPNPase at an m.o.i. of 50 p.f.u./cell. Transient transfection was conducted 12 hr post-infection using Lipofectamine-2000 transfection reagent (Invitrogen, Carlsbad, Calif.) and 1.2 μg of plasmid DNA per well that included 1 μg of pGL3Basic, 3 KB-Luc or 3 κBmut-Luc plasmids (30) and 0.2 μg of β-galactosidase-expression plasmid (pSV-β-gal; Promega, Madison, Wis.). For inhibition experiments, the cells were pre-treated with different inhibitors for 2 hr before transfection. Luciferase assays were performed 48 hr post-transfection using a Luciferase Reporter Gene Assay kit (Promega) according to the manufacturer's protocol. The β-galactosidase activity was determined using the Galacto-Light Plus kit (Tropix). Luciferase activity was normalized by β-galactosidase activity and the data from triplicate determinations were expressed as mean ± S.D.

Generation of Anti-OLD-35 Antibody. A C-terminal His-tagged old-35 protein was produced in a baculovirus expression system (Pharmingen, San Diego, Calif.) according to the manufacturer's instructions. The protein was purified by Ni-NTA agarose column and subsequently ion exchange chromatography. The purified protein was used to immunize chickens to generate anti-OLD-35 antibody (Genetel Laboratories, Madison, Wis.).

Cell Fractionation. Cells were harvested and the cytoplasm and nucleus were fractionated by the modified Schreiber's method as described (31). Briefly, the cells were washed with PBS and lysed for 10 min in Buffer A (10 mM HEPES pH 7.9, 10 mM KCl, 1.5 mM MgCl₂, 1 mM EDTA, 1 mM dithiothreitol, 0.1% Nonidet P-40) containing protease inhibitor cocktail (Roche, Mannheim, Germany). After centrifugation, the supernatant was saved as the cytoplasmic fraction and the pellet containing nuclei was lysed in Buffer B (20 mM HEPES pH 7.9, 400 mM KCl, 20% glycerol, 1 mM EDTA, 1 mM DTT) containing protease inhibitor cocktail (Roche) with one freeze-thaw cycle. After centrifugation the supernatant was collected as the nuclear fraction. Protein concentration was determined by Bio-Rad protein assay kit (Bio-Rad laboratories, Hercules, Calif.).

Electrophoretic Mobility Shift Assay (EMSA). EMSA was performed as described (31). The sequences of the consensus and mutated NF-κB probes are 5′-AGTTGAGGGGACTTTCCCAGGC-3′ (SEQ ID NO:7) and 5′-AGTTGAGGCGACTTTCCCAGGC-3′ (SEQ ID NO:8), respectively (Santa Cruz Biotechnology, Santa Cruz, Calif.). The probes were labeled using T4 polynucleotide kinase (Promega) and [γ³²P]ATP. Ten (10) μg of nuclear extract was incubated in a final volume of 10 μl containing gel shift binding buffer (Promega) for 10 min at room temperature and 20,000 cpm of labeled probed was added and incubated for another 20 min at room temperature. For competition studies, unlabeled probes were added 15 min before the labeled probe. For supershift analysis, the reaction mixture was incubated for 1 hr with 1 μl of antibody before the addition of the radiolabeled probe. The antibodies used were anti-p50, anti-p65, anti-p52 and anti-cRel (Santa Cruz; rabbit polyclonal). Free and bound DNA was separated on a 4% non-denaturating polyacrylamide gel in 0.5% Tris-borate-EDTA at a constant voltage of 165v. The gel was dried on filter paper and autoradiographed.

Preparation of Whole Cell Lysates and Western Blot Analysis. Whole cell lysates were prepared and Western blotting was performed as described (4). Briefly, cells were harvested in RIPA buffer containing protease inhibitor cocktail (Roche, Mannheim, Germany), 1 mM Na₃VO₄ and 50 mM NaF and centrifuged at 12,000 rpm for 10 min at 4° C. The supernatant was used as total cell lysate. Thirty μg of total cell lysate were used for SDS-PAGE and transferred to a nitrocellulose membrane. The primary antibodies used were anti-p65, anti-p50, IκB-α (Santa Cruz; rabbit polyclonal; 1:250), anti-EF1α (Upstate Biotechnology; mouse monoclonal; 1:1000), and anti-OLD-35⁵ (chicken; 1:10,000).

RNA Extraction and RT-PCR. Total RNA was extracted from the cells using Qiagen RNeasy mini kit (Qiagen, Hilden, Germany) according to the manufacturer's protocol. Two μg of total RNA was used for RT-PCR according to standard methods. The primers used were: IL-6 sense: 5′ CCACACAGACAGCCACTCACC 3′; (SEQ ID NO:9) IL-6 antisense: 5′ TGGCATTTGTGGTTGGGTCAG 3′; (SEQ ID NO:10) IL-8 sense: 5′ GGTGCAGAGGGTTGTGGAGAA 3′; (SEQ ID NO:11) IL-8 antisense: 5′ GCAGACTAGGGTTGCCAGATT 3′; (SEQ ID NO:12) GAPDH sense: 5′ ATGGGGAAGGTGAAGGTCGGAGTC 3′; (SEQ ID NO:13) GAPDH antisense; 5′ GCTGATGATCTTGAGGCTGTTGTC 3′ (SEQ ID NO:14).

Staining for Mitochondria and OLD-35. Live cells were loaded with 200 nM MitoTracker (Molecular Probes, Eugene, Oreg.) for 30 min according to the manufacturer's protocol, fixed in 3.7% formaldehyde in PBS for 15 min and permeabilized in 0.1% Triton X-100 in PBS. Immunocytochemistry was then performed by standard methods using chicken anti-old-35 antibody (1:1000) and FITC-conjugated anti-chicken secondary anibody (Genetel). The cells were visualized and the images were analyzed using a Zeiss confocal laser scanning microscope (LSM510) with a ×100 objective.

Monitoring ROS Production. Cells were stained with 2.5 μM hydroethidine (HE) and 5 μM 5,6-carboxy-2′,7′-dichlorofluorescein diacetate (DCFH-DA) in PBS for 30 min in the dark (32). Immediately after staining the cells were analyzed by flow cytometry (FACScan; Becton Dickinson, Mountain View, Calif.) and data were analyzed using CellQuest software, version 3.1 (Becton Dickinson). The cells were gated to exclude cell debris. For inhibition experiments, NAC was added 2 hr post-infection.

Human IL-6 and IL-8 ELISA. The levels of IL-6 and IL-8 were quantified by ELISA in culture supernatants using human IL-6 ELISA kit (Pierce Biotechnology, Rockford, Ill.) and human IL-8 ELISA kit (Pierce), respectively, according to the manufacturer's protocol.

Human Cytokine Arrays. The expression levels of 36 cytokines following Ad.vec and Ad. hPNPase^{old-}35 infection were analyzed in culture supernatants using the TranSignal™ human cytokine antibody array (Panomics, Redwood city, CA) according to the manufacturer's protocol.

Statistical analysis. Statistical analysis was performed using one-way analysis of variance (ANOVA), followed by Fisher's protected least significant difference analysis. A p value of <0.05 was considered as significant.

6.2 Results

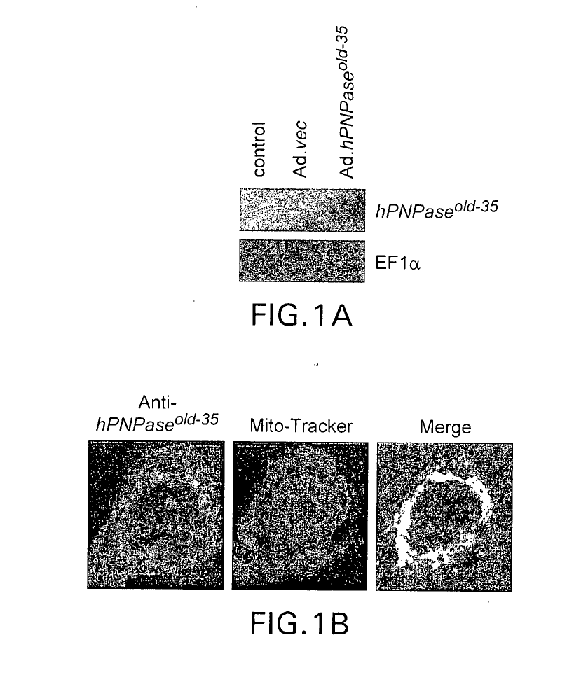

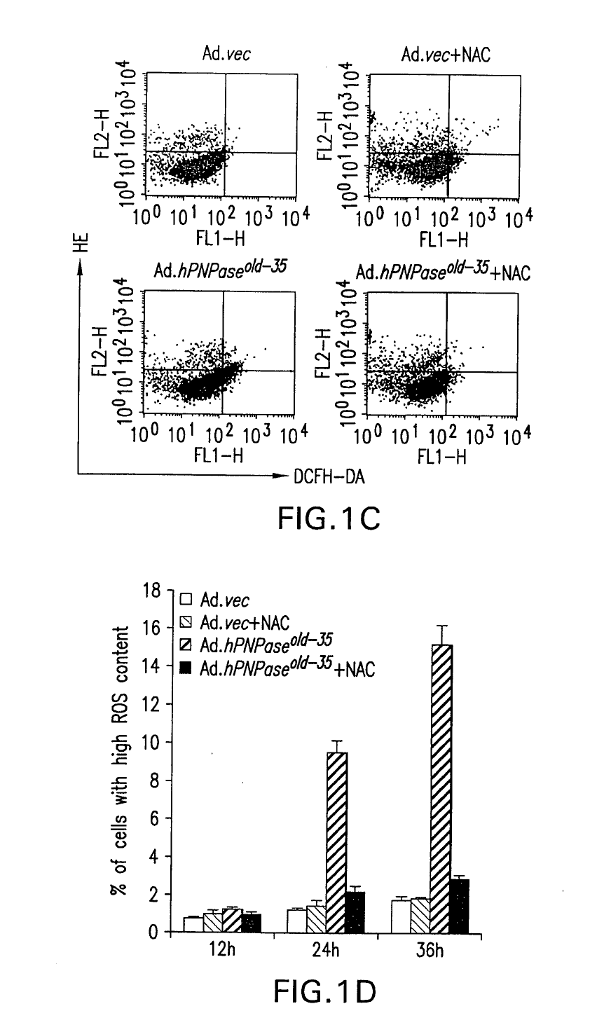

Ad.hPNPase^{old-35} Infection Generates OLD-35 Protein that Localizes to Mitochondria. The expression of OLD-35 in HeLa cells following Ad.hPNPase^{old-35} infection was analyzed two days post-infection by Western blot analysis. As shown in FIG. 1A, OLD-35 expression was detected only in Ad.hPNPase^{old-35}-infected cells indicating that Ad.hPNPase^{old-35} infection generates functional protein.

To confirm the subcellular localization of Ad.hPNPase^{old-35}-generated protein, cells were loaded with the mitochondria-specific dye MitoTracker and immunostained for OLD-35. Image analysis revealed that merging the signals generated from OLD-35 (green) and mitochondria (red) resulted in intense yellow color indicating overlapping distribution of the two signals (FIG. 1B). These findings strongly suggest that Ad.hPNPase^{old-35}-generated protein also localizes to mitochondria.

Infection with Ad.hPNPase^{old-35} Induces ROS. The levels of intracellular ROS following Ad.hPNPase^{old-35} infection were determined using two dyes, DCFH-DA and HE. Nonfluorescent DCFH-DA diffuses into cells where it is deacetylated to DCF that fluoresces on reaction with hydrogen peroxide or nitrous oxide (32). HE enters the cells and can be oxidized by superoxide or free hydroxyl radicals to yield fluorescent ethidium (32). Infection with Ad.hPNPase^{old-35} induced both DCFH-DA and HE fluorescence. Flow cytometry analysis of cellular fluorescence revealed a time-dependent increase in ROS production following Ad.hPNPaseold-35 infection in comparison to Ad.vec-infected cells (FIGS. 1C and 1D). The generation of ROS by Ad.hPNPaseold-35 was inhibited by treatment with a non-cytotoxic dose (20 mM) of a general antioxidant NAC which reduced the percentage of high ROS containing cells from ˜9% and ˜15% to ˜2% and ˜3% at 24 and 36 hr, respectively (FIG. 1D).

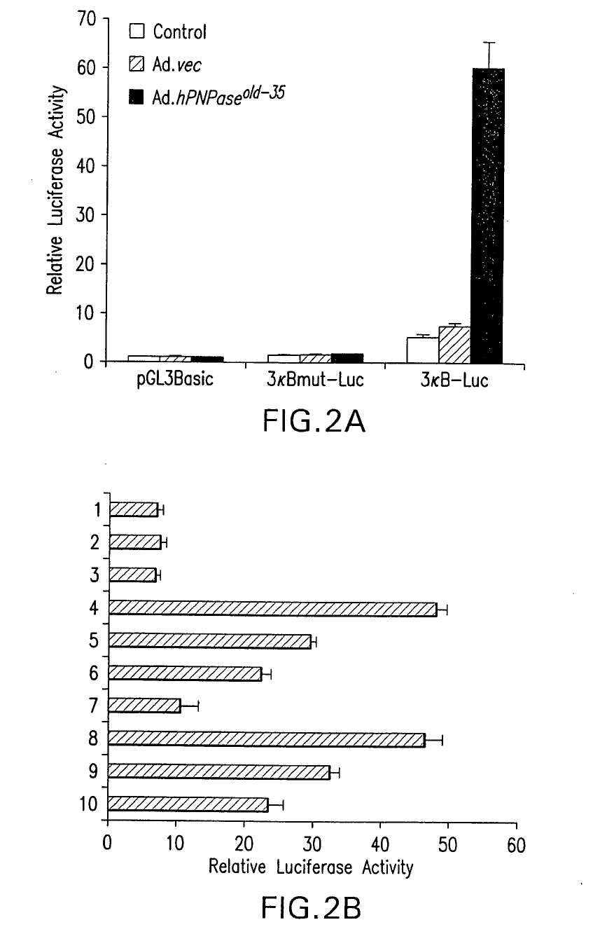

ROS Mediates Activation of the NF-κB Pathway following Ad.hPNPase^{old-35} Infection. To test whether Ad.hPNPase^{old-35} infection activates the NF-κB pathway a luciferase-based reporter gene assay was employed. HeLa cells were either uninfected or infected with Ad.vec or Ad.hPNPase^{old-35} and then transfected with either empty vector (pGL3Basic), 3κB-Luc containing 3 tandem NF-κB binding sites upstream of the luciferase gene or 3κBmut-Luc containing mutated NF-κB binding sites. As shown in FIG. 2A, cells transfected with either pGL3Basic or 3κBmut-Luc showed only basal luciferase activity under any experimental condition. In control and Ad.vec-infected cells transfection of 3κB-Luc increased basal activity over transfection of either pGL3Basic or 3κBmut-Luc, which is most likely a consequence of constitutive NF-κB DNA binding activity in HeLa cells. However, infection with Ad.hPNPase^{old-35} resulted in a 10-12-fold induction in relative luciferase activity in comparison to control or Ad.vec-infected cells indicating that Ad.hPNPase^{old-35} infection results in inducible activation of the NF-κB pathway (FIG. 2A). To evaluate the role of ROS in Ad.hPNPase^{old-35}-mediated NF-κB activation, HeLa cells were infected with Ad.hPNPase^{old-35}, then transfected with 3κB-Luc and treated with various doses of either NAC or Tiron. Luciferase activity was measured 48 hr post-transfection. As shown in FIG. 2B, treatment with increasing doses of either NAC or Tiron significantly decreased Ad.hPNPase^{old-35}-mediated NF-κB luciferase activity. NAC was much more potent than Tiron and in further studies NAC was used. These findings indicate that the induction of NF-κB upon Ad.hPNPase^{old-35} infection is mediated by the generation of ROS.

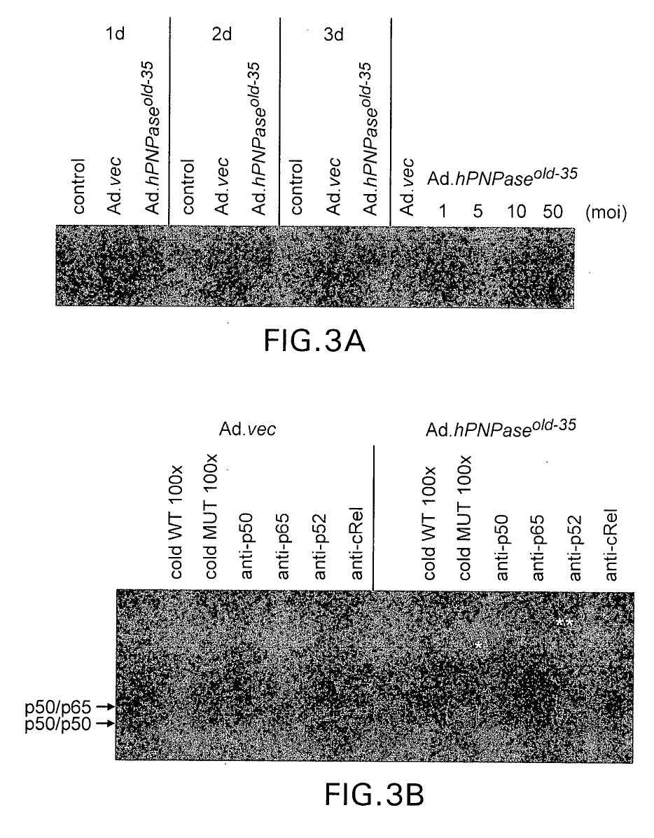

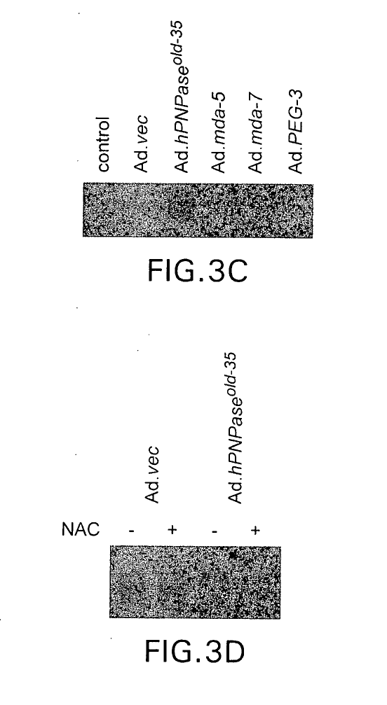

The activation of NF-κB upon Ad.hPNPase^{old-35} infection was further analyzed by EMSA using radiolabeled consensus NF-κB binding site as a probe and nuclear extracts from HeLa cells. As shown in FIG. 3A, in un-infected and Ad.vec-infected cells two shifted bands were observed at all time points. Following Ad.hPNPase^{old-35} infection, the fast migrating band started to disappear with a significant increase in the intensity of the slow migrating band. By 2 and 3 days post-Ad.hPNPase^{old-35} infection only the slow migrating band was detected and the intensity of the band was markedly higher in comparison to that in control and Ad.vec-infected cells. This change in DNA-binding pattern could be observed with as little as 5 m.o.i. of Ad.hPNPase^{old-35} infection and at 50 m.o.i., the intensity of the shifted band was significantly enhanced.

To characterize the shifted bands, competition assays using excess amounts of cold wild type and mutated probes and supershift assays using antibodies against different subunits of NF-κB were employed. As shown in FIG. 3B, all the shifted bands were completely eliminated by a 100-fold excess of cold wild type probe but not by cold mutated probe indicating that these shifted bands are specific for NF-κB. The anti-p50 antibody supershifted both the slow and fast migrating bands while anti-p65 antibody supershifted only the fast migrating band in Ad.vec-infected cells. The intense slow migrating band in Ad.hPNPase^{old-35}-infected samples was supershifted by both anti-p50 and anti-p65 antibodies, but not by anti-p52 or anti-cRel antibodies. These findings demonstrate that under basal condition, both p50/p50 homodimers and p50/p65 heterodimers bind to the NF-κB probe. However, upon Ad.hPNPase^{old-35} infection, the binding pattern changed with the p50/p50 homodimer disappearing and the binding of the p50/p65 heterodimer increasing markedly. These findings also indicate that OLD-35 is a potent activator of NF-κB, since it promotes increased binding of the potent transcriptional activator p50/p65.

To confirm that the induction of NF-κB by Ad.hPNPase^{old-35} is specific and not mediated by non-specific events such as protein overload or additional adenoviral proteins, that might activate NF-κB, HeLa cells were infected with three additional replication incompetent adenovirus constructs expressing functionally different genes and NF-κB binding was analyzed by EMSA. The constructs included Ad.mda-5 that expresses mda-5, an interferon-inducible putative RNA helicase (33), Ad.mda-7 that expresses the apoptosis-inducing cytokine mda-7/IL-24 (34) and Ad.PEG-3, that express PEG-3 which is involved in tumor progression (35). As shown in FIG. 3C, infection with only Ad.hPNPase^{old-35} and not with any other adenoviral construct resulted in a significant increase in the binding of NF-κB confirming the specificity of this induction.

The involvement of ROS in mediating increased NF-κB DNA binding by Ad.hPNPase^{old-35} was also evaluated by EMSA. As shown in FIG. 3D, treatment with NAC significantly reduced the intensity of the shifted band induced by Ad.hPNPase^{old-35} infection, which further strengthens the conclusion that ROS acts as the second messenger to activate NF-κB following Ad.hPNPase^{old-35} infection.

The levels of p50 and p65 subunits of NF-κB and its inhibitor IκBcc were analyzed in cytoplasmic and nuclear extracts following Ad.hPNPase^{old-35} infection. As shown in FIG. 4A, the levels of both p65 and p50 proteins began decreasing in the cytoplasmic extract of cells 2 days post-Ad.hPNPase^{old-31} infection, whereas the level of p65 protein started increasing in the nuclear extract of Ad.hPNPase^{old-35}-infected cells 2 days post-infection. This effect was not apparent in control or Ad.vec-infected cells (FIG. 4B), indicating that Ad.hPNPase^{old-35} infection resulted in translocation of p65 from the cytoplasm to the nucleus. The basal p50 protein level in the nucleus was quite high and this level was not modulated significantly following Ad.hPNPase^{old-35} infection. The level of IκBα significantly decreased in the cytoplasmic extract with Ad.hPNPase^{old-35} infection, but not in control or after Ad.vec infection (FIG. 4A). Both the degradation of IκBα and nuclear translocation of p65 by Ad.hPNPase^{old-35} infection were inhibited by treatment with NAC (FIG. 4C) indicating that hPNPase^{old-35}-induced generation of ROS plays a pivotal role in activating NF-κB.

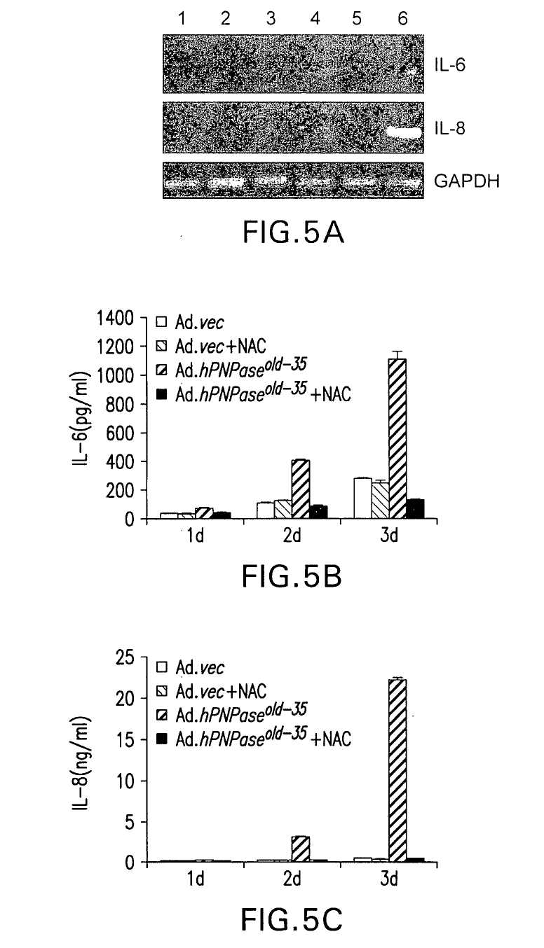

IL-6 and IL-8 are Induced by Ad.hPNPase^{old-35} Infection. Expressions of mRNAs of IL-6 and IL-8, two NF-κB target genes, were analyzed by RT-PCR following Ad.hPNPase^{old-35} infection. A time-dependent increase in the expressions of both IL-6 and IL-8 mRNAs occurred from two days post-infection with Ad.hPNPase^{old-35}, but not with Ad.vec infection (FIG. 5A). The expression of the mRNA of the housekeeping gene GAPDH did not change under any experimental condition.

ELISA assays quantified secretion of IL-6 and IL-8 protein following Ad.hPNPase^{old-35} infection (FIG. 5B, 5C). There was a significant time-dependent increase in secreted IL-6 and IL-8, the latter markedly more robust, in the culture supernatant from two days post-infection with Ad.hPNPase^{old-35} as compared to that in control or Ad.vec-infected cells. Treatment with NAC markedly inhibited Ad.hPNPase^{old-35}-induced secretion of both IL-6 and IL-8 (FIG. 5B, 5C).

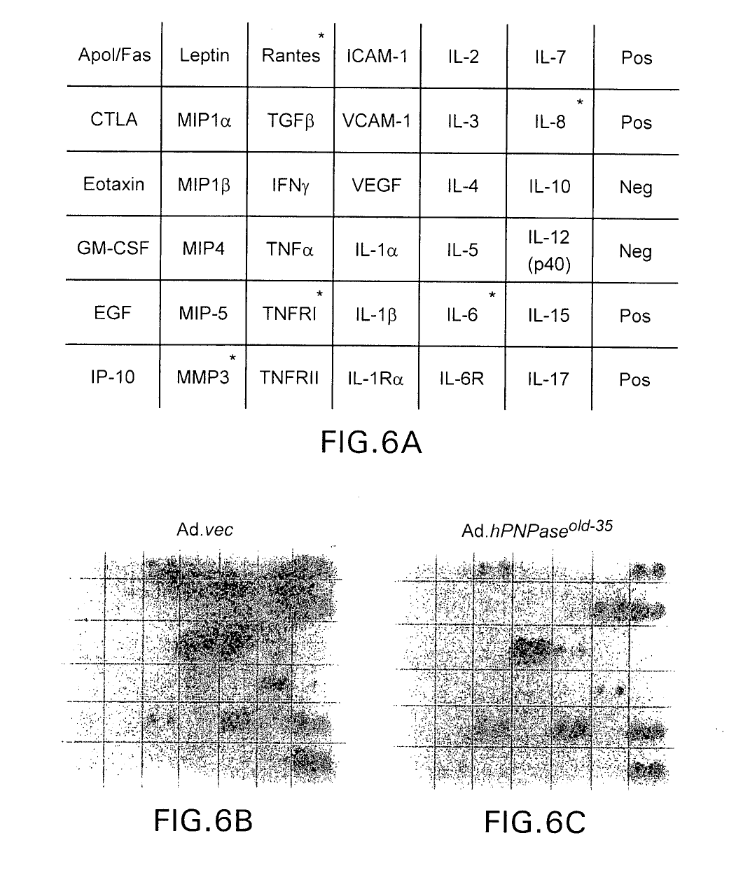

Analysis of Cytokine Expression Profiles following Ad.hPNPase^{old-35} Infection. Since Ad.hPNPase^{old-35} infection induced two potently pro-inflammatory cytokines, IL-6 and IL-8, the induction of other cytokines in culture supernatants two days post-infection were also tested using a human cytokine antibody array that analyzes the expression levels of 36 cytokines. Ad.hPNPase^{old-35} infection had a very specific cytokine induction profile resulting in marked upregulation of IL-8, moderate elevation of IL-6 and TNFR1 and upregulation of RANTES and MMP-3 to a lesser extent (FIG. 6).

6.3 Discussion

The foregoing demonstrates that OLD-35 activates the NF-κB pathway via the generation of ROS in HeLa cells. ROS is involved in the induction of a senescent phenotype characterized by irreversible growth arrest (10). Overexpression of Old-35 induces a senescence-like growth arrest and also generates ROS. Moreover, inhibition of ROS impedes the induction of NF-κB-responsive genes.

Contrasting results have been obtained regarding NF-κB binding activity during aging. No difference was observed in NF-κB binding between senescent and pre-senescent human fibroblasts as a function of in vitro replicative senescence (36). A reduced NF-κB activation in T cells from aged humans and mice has been reported (37). On the other hand, an increase in constitutive NF-κB DNA binding in older animals over young animals has been demonstrated in multiple studies (14-21). A gradual rise in ROS was evident in kidneys from Fischer rats from 6 to 24 months of age and this increase correlated with an age-dependent augmentation in binding of p50/p65 NF-κB, IκBot degradation, p65 nuclear translocation and elevated expression of cyclogenase-2 (COX-2), an NF-κB-responsive enzyme involved in pro-inflaimmatory prostanoid synthesis (16). Vascular smooth muscle cells from 18-month old rats showed considerably higher p50/p65 NF-κB DNA binding than that from new-born rats which correlated with increased expression of inducible nitric oxide synthase and intracellular adhesion molecule-1, two pro-inflammatory molecules, in old smooth muscle cells upon inflaimmatory stimulation (17). A similar age-dependent elevation in NF-κB DNA binding has been reported in mouse and rat liver and heart and in rat brain (14, 19). In these tissues an age-dependent rise in the levels of NF-κB subunits, such as p50, p52 and p65, could be observed. However, no change in the level of IκBr was detected. From these studies it might be inferred that tissue-specific regulatory mechanisms may be involved in NF-κB activation during senescence. Although NF-κB activation requires degradation of IκBα, IκBα itself is an NF-κB-responsive gene (22, 38). During acute activation of NF-κB by TNFα or related stimuli, there is an initial decrease in the cytoplasmic IκBα level followed by gradual restoration because of NF-κB-mediated transcription (38). Infection with Ad.hPNPase^{old-35} resulted in a persistent decrease in the cytoplasmic IκBcc level, indicating that even though NF-κB is activated by OLD-35 there might be an additional regulatory mechanism of IκBc transcription during senescence as compared to acute stimuli. A recent study has shown the lack of involvement of ROS in NF-κB activation by acute stimuli such as TNFα. (39). It is possible that in a state of chronic oxidative stress, such as senescence, ROS plays a role in activating NF-κB. Another intriguing observation is the selective induction of NF-κB-responsive genes by OLD-35 (FIG. 6) indicating that in addition to the primary transactivation of NF-κB by OLD-35 there might be a secondary level of regulation that targets the transactivation of specific NF-κB-target genes.

What is the significance of induction of NF-κB-responsive genes by OLD-35 in the context of senescence? Ad.hPNPase^{old-35} infection results in the upregulation of pro-inflammatory cytokines via activation of NF-κB. By turning on pro-inflammatory cytokines, NF-κB functions as a central transcription factor for the development of chronic inflammatory diseases (40). Gene expression analysis by microarray in human hepatic stellate cells confirms that replicative senescence in these cells is associated with a pronounced inflammatory phenotype characterized by upregulation of pro-inflammatory cytokines, including IL-6 and IL-8 (41). An aging-induced pro-inflammatory shift in cytokine expression profile has been observed in rat coronary arteries (42). Several studies have documented increased blood level of pro-inflammatory cytokines such as IL-1, IL-6, TNFα and IL-8 in aged individuals as compared to young individuals (43). The onset and course of a spectrum of age-associated diseases, such as cardiovascular disease, osteoporosis, arthritis, type 2 diabetes, Alzheimer's disease, certain cancers, periodontal disease, frailty and functional decline, might be associated with the production of pro-inflammatory cytokines (44, 45). Multiple studies have established an association between elevated levels of IL-6 and diseases of old age. IL-6 induces the production of C-reactive protein (CRP), an important risk factor for myocardial infarction (45). High concentrations of CRP predict the risk of future cardiovascular disease in apparently healthy men (45). IL-8 plays a crucial role in initiating atherosclerosis by recruiting monocytes/macrophages to the vessel wall, which promotes atherosclerotic lesions and plaque vulnerability (46). Elevated levels of IL-6 and CRP predict the development of type 2 diabetes in healthy women (47). In another study, elevated serum IL-6 levels predicted future disability in older adults especially by inducing muscle atrophy (48). IL-6 and CRP also play a pathogenic role in several diseases such as osteoporosis, arthritis and congestive heart failure all of which have increasing incidence with age (48). Moreover, increased serum levels of IL-6 and IL-8 have been detected in patients with chronic obstructive pulmonary diseases and chemokines such as IL-8 and RANTES play important roles in the pathogenesis of these diseases (49, 50). Various inflammatory mediators, such as IL-1, TNF-α, IL-6, IL-8, RANTES, MMP-3 are responsible for chronic inflammatory rheumatoid diseases, such as osteoarthritis and rheumatoid arthritis both of which occur during aging (51). The observation that the senescence-associated molecule OLD-35 induces pro-inflammatory cytokines which are intimately involved in the development of aging-associated diseases are consistent with involvement of OLD-35 in these pathological processes.

### 7. EXAMPLE

### Defining the Domains of OLD-35

7.1 Materials and Methods

Cell lines and culture conditions: The human metastatic melanoma cell line HO-1 and the human embryonic kidney cell line HEK-293 were cultured in Dulbecco's Modified Eagle Medium (DMEM) supplemented with 10% fetal bovine serum, 50 units/ml penicillin G and 50 μg/ml streptomycin at 37° C. in 5% CO₂ and 100% relative humidity. HO-1 cells were cultured with IFN-β at a dose of 1000 OLD-35 units/ml for 2 days to detect hPNPase protein.

Plasmid Construction: All plasmids were constructed in a backbone of pcDNA3.1+(Hygro) (Invitrogen, Carlsbad, Calif.) into the NheI and BamHI sites and all the constructs contained a COOH-terminal Hemagglutinin (HA)-tag. phPNPase^{old-35}, expressing the full-length hPNPase^{OLD-}35 protein, was amplified by PCR using the cloned hPNPase^{old-35} cDNA as a template (75) and primers sense (1) 5′-GCTAGCATGGCGGCCTGCAGGTAC-3′ (SEQ ID NO:33) and anti-sense (1) 5′-GGATCCTCAAGCGTAATCTGGAACATCGTATGGGTACTGAGAATTAGATG ATGA-3′ (SEQ ID NO:34). pΔRPH1 was created by PCR using primers sense (2) 5′-GCTAGCATGCCTTGGAATGGACCTGTTGGG-3′ (SEQ ID NO:35) and antisense (1). pΔRPH2 was cloned in two steps. First, a 3′ PCR fragment was amplified by PCR using primers sense (3) 5′-GTTAACATGGATTCAGGGGTTCCAATT-3′ (SEQ ID NO:36) and antisense (1) and ligated to pGEMT-easy vector (Promega, Madison, Wis.) by TA-cloning. This fragment was digested with HpaI and BamHI and ligated into HpaI and BamHI-digested phPNPase^{old-35}. pΔRPH1+2 was generated by PCR using primer sense (4) 5′-GCTAGCATGGATTCAGGGGTTCCAATT-3′ (SEQ ID NO:37) and antisense (1). pΔC-term was generated by PCR using primers sense (1) and antisense (2) 5′-GGATCCTCAAGCGTAATCTGGAACATCGTATGGGTACTGCAACAGCAGAT GAAATTGG-3′ (SEQ ID NO:38). The authenticity of all the constructs was confirmed by sequencing. The PCR fragments were first cloned into the vector pGEMT-easy by TA-cloning and then transferred to pcDNA3.1+(Hygro). The c-myc expression plasmid [p290-myc(2,3)] was provided by Dr. Riccardo Dalla-Favera (Columbia University Medical Center, N.Y.).

Virus Construction and Infection Protocol: The construction of hPNPase^{old-35} expressing replication-defective Ad.hPNPase^{old-35} was performed by cloning the transgene into a shuttle vector (p0TgCMV) and then performing homologous recombination of the shuttle vector with E1 and E3 region deleted parental adenoviral vector in E. coli as described previously (69, 75). A similar method was employed to generate Ad.ΔRPH1, Ad.ΔRPH2, Ad.ΔRPH1+2 and Ad.ΔC-term. The transgene was digested from the pGEMT-easy vector with NotI and ligated into the NotI site of p0TgCMV. The direction of the cloning was confirmed by restriction enzyme digestion and sequencing. The empty adenoviral vector (Ad.vec) was used as a control. The Ad was propagated in HEK293 cells (69), purified by BD AdenoX™ Virus Purification Kit (BD Biosciences, Palo Alto, Calif.) and viral titer was determined by measuring O.D. at 260 nm and using BD AdenoX™ rapid titer kit (BD Biosciences). Ad infection was performed 24 h after cell plating in one-fifth the volume of the original culture medium in a serum-free condition for 2 h with rocking the plates several times (69).

Colony formation assays: HO-1 cells were plated at a density of 3×10⁵ cells per 6-cm dish and 24 h later were infected with different Ad at an m.o.i. of 10, 20 and 50 pfu/cell. For colony formation assays with c-myc overexpression, HO-1 cells were plated at a density of 3×10⁵ cells per 6-cm dish and 24 h later were transfected with 5 μg of either empty vector or p290-myc(2,3) using Superfect® (Qiagen, Hilden, Germany) transfection reagent according to the manufacturer's protocol. After 36 h, the cells were infected with different Ad at an m.o.i. of 50 pfu/cell. Six h after infection, the cells were trypsinized, counted and 1 cells were plated in 6-cm dishes. Colonies>50 cells were counted after 2 weeks.

Western Blot Analysis. Western blotting was performed as previously described (84). Briefly, cells were harvested in RIPA buffer (1×PBS, 1% NP-40, 0.5% sodium deoxycholate, 0.1% SDS) containing protease inhibitor cocktail (Roche, Mannheim, Germany), 1 mM Na₃VO₄ and 50 mM NaF and centrifuged at 12,000 rpm for 10 min at 4° C. The supernatant was used as total cell lysate. Thirty μg of total cell lysate were used for SDS-PAGE and transferred to a nitrocellulose membrane. The primary antibodies included: from Santa Cruz Biotechnology, Santa Cruz, Calif.: Myc (1:200; mouse monoclonal), Max (1:200; rabbit polyclonal), Mad1 (1:200; rabbit polyclonal), p21 (1:200; rabbit polyclonal), p27 (1:200; rabbit polyclonal), CDK2 (1:250; rabbit polyclonal); from BD Biosciences: p16 (1:500; mouse monoclonal), Rb (1:500, mouse monoclonal), actin (1:1000, mouse monoclonal), cytochrome c (1:1000, mouse monoclonal), cyclin B1 (1:5000; mouse monoclonal); anti-hPNPase^{OLD-35} (1:10,000; chicken polyclonal) (82); anti-HA (1:1000; mouse monoclonal; Covance Research Products, Inc, Berkeley, Calif.); and EF1α(1:1000; mouse monoclonal; Upstate Biotechnology, Waltham, Mass.).

Immunofluorescence analysis: HO-1 cells were plated on chamber slides (Falcon 4102; Becton Dickinson; Franklin Lakes, N.J.) and infected with different Ad. After 36 h, the cells were loaded with 250 mM MitoTracker (Molecular Probes, Eugene, Oreg.) for 30 min at 37° C., fixed with 3.7% formaldehyde for 15 min at 37° C. and permeabilized with 0.1% Triton-X-100 in PBS for 5 min at room temperature (RT). The cells were blocked with PBS containing 10% normal rabbit serum for 2 h at RT and incubated in the blocking solution containing anti-HA antibody (1:200) overnight at 4° C. After washing in PBS, the cells were incubated in the blocking solution containing anti-mouse-FITC (1:200) for 2 hi at RT, washed again in PBS, mounted and visualized using a Zeiss confocal laser scanning microscope (LSM510) and a 40× objective. In case of double immunofluorescence studies for hPNPase^{OLD-35} and p27^{KIP-1}, HO-1 cells were plated on chamber slides, infected with Ad.hPNPase^{old-35} and 48 h later they were fixed, permeabilized and blocked with PBS containing 5% normal goat serum. The cells were incubated first with anti-p27 antibody and Alexa Fluor 594 (red) goat anti-rabbit IgG (Molecular probes) followed by anti-hPNPase^{OLD-35} antibody and rabbit anti-chicken-FITC (Genetel Laboratories, Madison, Wis.). Image analysis was performed using a Zeiss confocal laser scanning microscope (LSM510) and a 40× objective.

Cell Fractionation: 2×10⁷ cells were harvested by trypsinization and mitochondrial and cytoplasmic fractions were separated using a Mitochondria Isolation kit (Pierce, Rockford, Ill.) according to the manufacturer's protocol.

Assay for Senescence Associated β-galactosidase (SA-β-gal) activity: SA-β-gal activity was assayed 4 days after infection with different Ad at an m.o.i. of 50 pfu/cell (61). The cells were fixed with 2% formaldehyde+0.2% glutaraldehyde and then stained with X-gal (1 mg/ml) in 40 mM citric acid/Na phosphate buffer (pH 6.0) containing 5 mM potassium ferrocyanide, 5 mM potassium ferricyanide, 150 mM NaCl and 2 mM MgCl₂. After the development of color the cells were washed with PBS and methanol, air-dried and micro-photographed. Quantification of SA-β-gal positive cells was determined by counting at least 1,000 cells for each group.

Cell cycle analysis: Cell cycle was analyzed at 1, 2 and 3 days post-infection. Cells were harvested, washed in PBS and fixed overnight at −20° C. in 70% ethanol. Cells were treated with RNase A (1 mg/ml) at 37° C. for 30 min and then with propidium iodide (50 μg/ml). Cell cycle was analyzed using a FACS Calibur flow cytometer, and data was analyzed using CellQuest software (Becton Dickinson, San Jose, Calif.).

Cell sorting analysis: HO-1 cells were transfected with either empty vector or c-myc expression plasmid and infected the next day with either Ad.vec or Ad.hPNPaseold-35 at an m.o.i. of 50 pfu/cell. Two days after infection, live cells were incubated with 5 μg/ml of Hoechst 33342 (Molecular Probes) for 1 h in the dark. After trypsinization, cells were resuspended at a concentration of 1×10⁷ cells/ml for sorting. Cells were sorted based on the amount of DNA by defining three regions for sorting: one for G₁, one for S and one for G₂+M using BDFACSAria (BD Biosciences) equipped with a UV laser required for Hoechst 33342 excitation. The separated cells (at least 1×10⁶ cells from each sorted population) were collected, protein was extracted and Western blot analysis was performed.

RNA Isolation and Northern Blot Analysis: Total RNA was extracted from the cells using Qiagen RNeasy mini kit (Qiagen) according to the manufacturer's protocol and Northern blotting was performed as described (84). The cDNA probes used were a 500-bp fragment from human c-myc, full-length human GADD34 and full-length human GAPDH.

In vitro Translation and in vitro mRNA Degradation Assays: In vitro translation was performed using the TNT coupled Reticulocyte Lysate Systems (Promega) using the plasmids pcDNA3.1+(Hygro) as a control, GADD153 expression plasmid, phPNPase^{old-35}, pΔRPH1, pΔRPH2, pΔRPH1+2 and pΔC-term according to the manufacturer's protocol. Five fig of total RNA from HO-1 cells were incubated with 5 μl of each in vitro translated protein at 37° C. from 0.5 to 2 h. The RNA was repurified using the Qiagen RNeasy mini kit (Qiagen) and Northern blotting was performed.

CDK Activity Assay: Cells were harvested in RIPA buffer and 500 μg of protein were incubated overnight at 4° C. with anti-CDK2 antibody and then with protein A agarose for 1 h at 4° C. The agarose beads were spun down at 10,000 g for 5 min, washed three times in RIPA buffer and once in kinase buffer. The immunoprecipitated material was employed for kinase assays using Histone H1 (Upstate Biotechnology) as substrate and [γ-³²P]ATP (Amersham, Piscataway, N.J.) in a kinase buffer containing 25 mM Tris-HCl pH 7.5, 150 mM NaCl, 18 mM MgCl₂ and 1 mM DTT for 30 min at 30° C. Following the reaction the samples were subjected to 15% SDS-PAGE, the gel was dried, exposed to x-ray film and densitometric analysis was performed.

Statistical analysis: Statistical analysis was performed using one-way analysis of variance (ANOVA), followed by Fisher's protected least significant difference analysis.

7.3 Results

Polynucleotide phosphorylases are highly conserved across species ranging from bacteria, plants to mammals (74). They have a conventional structure containing RNAse PH domains and RNA binding domains (88, 89). FIG. 12A (top panel) shows the different domains of hPNPase^{OLD-35}. The hPNPase^{OLD-35} protein contains 783 amino acid residues. The first 45 a.a. contains a mitochondrial localization signal. Interestingly, the bacterial PNPase does not contain such a signal, whereas the plant PNPase contains a chloroplast localization signal suggesting possible evolutionary divergence of this gene in eukaryotes. hPNPase^{OLD-35} contains two RNAse PH(RPH) domains, involved in RNA degradation, one at a.a. 52-183, the other at a.a. 366-501. Between the two RPH domains there is an α-helix at a.a. 289-363 that is unique for PNPase and is involved in RNA binding. There are two RNA binding domains at the COOH-terminal of the molecule, the KH domain is at a.a. 605-667 and the S1 domain is at a.a. 676-750.

In order to comprehend the involvement of these domains in mediating the hPNPase^{old-35}-induced senescent phenotype, a number of deletion mutants were created and replication-incompetent adenoviruses (Ad) expressing these deletion mutants were generated (FIG. 12A). All the constructs were tagged with a C-terminal Hemagglutinin (HA)-epitope (YPYDVPDYA) (SEQ ID NO:39) for monitoring the site and level of expression of the proteins. Ad.hPNPase^{old-35} contains the complete ORF of hPNPase^{old-35}, Ad.ΔRPH1 contains a.a. 183-783 lacking the mitochondrial localization signal and RPH1 domain, Ad.ΔRPH2 contains a.a. 1-202 and 496-783 lacking the PNPase and RPH2 domains, Ad.ΔRPH1+2 contains a.a. 496-783 lacking the mitochondrial localization signal, both RPH domains and PNPase domain and Ad.ΔC-term contains a.a. 1-507 lacking the KH and S1 RNA binding domains. The predicted molecular weight of the proteins generated from these constructs are 86-, 67-, 54-, 31- and 55-kDa for hPNPase^{OLD-35}, ΔRPH1, ΔRPH2, ΔRPH1+2 and ΔC-term, respectively, which was confirmed by Western blotting

It was considered important to initially determine if the level of expression of hPNPase^{old-35} resulting from adenoviral delivery of this gene was comparable to the level of endogenous protein induced following IFN-β treatment. To address this issue, HO-1 cells were infected with either Ad.vec (control empty Ad) or with Ad.hPNPase^{old-35} at an m.o.i. of 50 pfu/cell or treated with 1000 units/ml of IFN-βand the expression of hPNPase^{OLD-35} was analyzed two days later by Western blot analysis using anti-hPNPase^{old-35} antibody (82). Treatment with IFN-β resulted in marked induction of hPNPase^{OLD-35} and the level of the protein generated upon Ad.hPNPase^{old-35} infection was ˜2-fold more than that with IFN-β treatment (FIG. 12B). These findings indicate that Ad.hPNPase^{old-35} generates hPNPase^{OLD-35} protein that is within a physiological range and the effects observed with Ad.hPNPase^{old-35} infection represent potentially physiologically relevant events.

To analyze the involvement of the different domains in hPNPase^{old-35}-induced growth inhibition and senescence, HO-1 human melanoma cells were infected with the different Ad at an m.o.i. of 10, 20 and 50 pfu/cell and colony formation assays were performed. As a control, cells were either uninfected or infected with Ad.vec at an m.o.i. of 50 pfu/cell. As shown in FIG. 12C, infection with Ad.hPNPase^{old-35}, Ad.ΔRPH1, Ad.ΔRPH2 and Ad.ΔC-term resulted in a dose-dependent inhibition in growth. At an m.o.i. of 50 pfu/cell colony formation was reduced by 60%, 54%, 51% and 51% upon infection with Ad.hPNPase^{old-35}, Ad.ΔRPH1, Ad.ΔRPH2 and Ad.ΔC-term, respectively. On the other hand, infection with Ad.vec or Ad.ΔRPH1+2 did not result in any significant growth inhibition. These findings confirm that retention of only the C-terminal RNA binding domains are not adequate, whereas either of the RPH domains is sufficient for mediating growth inhibition. The observation that Ad.ΔC-term also has potent growth inhibiting properties indicates that the PNPase RNA binding domain might be sufficient for RNA binding and subsequent RPH activation.

To determine the senescence-inducing properties of these deletion mutants, HO-1 cells were infected with the different Ads and monitored for changes in cell morphology and senescence-associated β-galactosidase (SA-β-Gal) activity, a characteristic marker of senescence (61). Infection with Ad.hPNPase^{old-35}, Ad.ΔRPH1, Ad.ΔRPH2 and Ad.ΔC-term, but not Ad.vec and Ad.ΔRPH1+2, resulted in typical morphological changes in HO-1 cells (FIG. 12D). Large, flattened cells that stained for SA-β-Gal were observed (white arrows). In Ad.vec and Ad.ΔRPH1+2-infected cells 7 and 6% of the cells, respectively, stained positive for SA-β-Gal while in Ad.hPNPase^{old-35}, Ad.ΔRPH1, Ad.ΔRPH2 and Ad.ΔC-term-infected cells 35, 28, 45 and 29% of the cells, respectively, displayed SA-β-Gal-positivity (FIG. 12E). These findings indicate that similar to growth inhibition the morphological and biochemical changes, characteristic of senescence and induced by hPNPase^{old-35}, also require at least one of the two RPH domains.

Senescence is associated with arrest of cell cycle especially at the G₁ phase, with a decrease in the S phase indicative of inhibition of DNA synthesis (86). Cell cycle analysis was performed in HO-1 cells infected with the different Ads. A time-dependent change in the cell cycle pattern was observed (FIG. 13A-D). Three days after infection, 51% of the cells were in the G₁ phase following Ad.vec and Ad.ΔRPH1+2 infection, respectively. At the same time point, 73%, 71%, 72% and 69% of the cells were in the G₁ phase following Ad.hPNPase^{old-35}, Ad.ΔRPH1, Ad.ΔRPH2 and Ad.ΔC-term infection, respectively. Similarly, while 17% and 15% of the cells were in the S phase following Ad.vec and Ad.ΔRPH1+2 infection, respectively, 7%, 9%, 8% and 8% of the cells were in the S phase following Ad.hPNPase^{old-35}, Ad.ΔRPH1, Ad.ΔRPH2 and Ad.ΔC-term infection, respectively. These findings corroborate the findings of SA-β-gal staining that growth arrest in the G₁ phase and inhibition of DNA synthesis induced by hPNPase^{old-35} also requires the RPH domain.

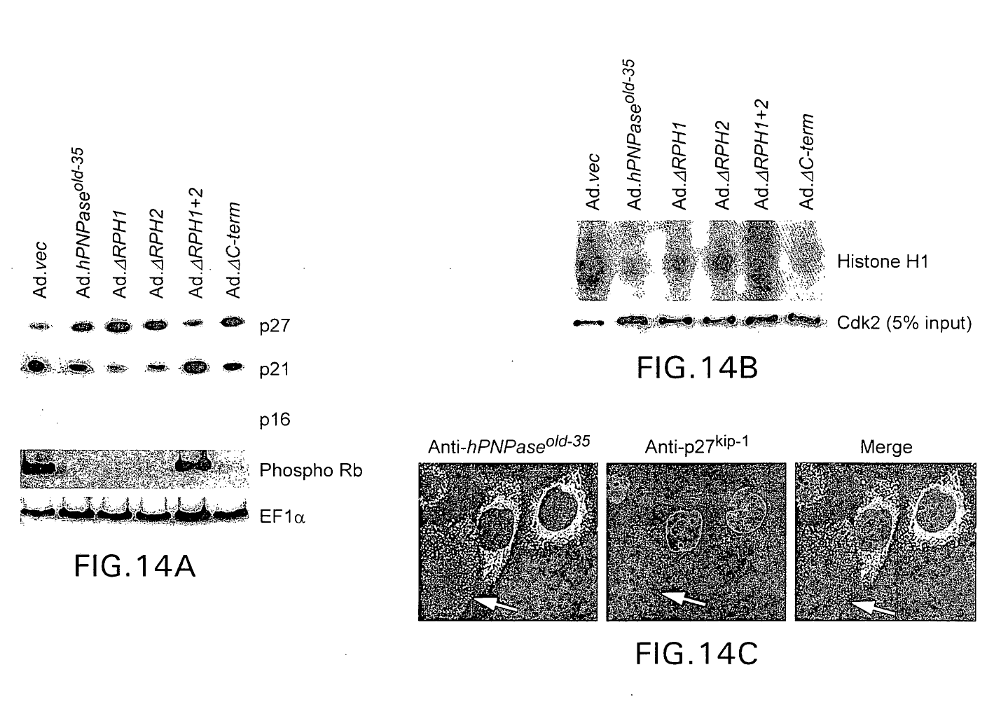

We next investigated the expressions of proteins that regulate the progression of the cell cycle beyond the G₁ phase by Western blot analysis. There was a significant increase in p27^{KIP1} and a decrease in p21^{CIP1/WAF-1/MDA-6} upon infection with Ad.hPNPase^{old-35}, Ad.ΔRPH1, Ad.ΔRPH2 and Ad.ΔC-term, but not with Ad.vec and Ad.ΔRPH1+2 (FIG. 14A). No p16^{INK4A} protein was detected in HO-1 cells, which is due to the fact that a majority of melanomas have genomic abnormalities in the p16^{INK4A} gene (56). The level of phosphorylated Rb decreased significantly upon infection with Ad.hPNPase^{old-35}, Ad.ΔRPH1, Ad.ΔRPH2 and Ad.ΔC-term. These findings show that the increase in the level of p27^{KIP1} inhibits cyclin dependent kinase activity resulting in hypophosphorylation of Rb and arrest of cell cycle in the G₁ phase. This possibility was confirmed by assaying for CDK2 activity by in vitro kinase assays using Histone H1 as a substrate. Cell lysates obtained from Ad.vec and Ad.ΔRPH1+2 had high CDK2 kinase activity that was markedly reduced in cell lysates obtained from Ad.hPNPase^{old-35}, Ad.ΔRPH1, Ad.ΔRPH2 and Ad.ΔC-term-infected cells (FIG. 14B). The level of CDK2 itself was similar in all the samples indicating that the decrease in the CDK2 activity is not because of a decrease in CDK2 itself, but a consequence of upregulation of the CDKI p27^{KIP1} (FIG. 14B). The relationship between Ad.hPNPase^{old-35} infection and p27^{KIP1} upregulation was further documented by double immunofluorescence analysis. The cells expressing no hPNPase^{OLD-35} showed basal level of p27 ^{KIP1} expression (FIG. 14C, arrow) while hPNPase^{OLD-35}-expressing cells displayed higher levels of p27^{KIP1} (FIG. 14C).

It was documented previously that downregulation of c-myc plays a significant role in mediating hPNPase^{old-35}-induced growth inhibition (83). Considering this possibility, the involvement of the domains of hPNPase^{old-35} in regulating c-myc expression was analyzed. Infection with Ad.hPNPase^{old-35}, Ad.ΔRPH1, Ad.ΔRPH2 and Ad.ΔC-term, but not with Ad.vec and Ad.ΔRPH1+2, resulted in ˜50% reduction in c-myc mRNA level 3 days post-infection (FIG. 15A-B). This reduction was also reflected at the level of the MYC protein (FIG. 15C). The expression of MYC protein decreased while that of MAD-1 protein increased significantly following Ad.hPNPase^{old-35}, Ad.ΔRPH1, Ad.ΔRPH2 and Ad.ΔC-term, but not with Ad.vec and Ad.ΔRPH1+2 infection. The level of MAX and EF1α remained unchanged under all treatment protocols. These findings indicate that the RPH domains of hPNPase^{old-35} are required to downregulate c-myc expression, which results in a switch from a MYC-MAX transcriptional activator to MAD-1-MAX transcriptional repressor.

To support the observations of these expression studies, c-myc was overexpressed in HO-1 cells and these cells were infected with the different Ads and colony-forming ability was determined. Infection with Ad.hPNPase^{old-35}, Ad.ΔRPH1, Ad.ΔRPH2 and Ad.ΔC-term decreased colony formation by 75%, 73%, 79% and 74%, respectively in comparison to infection with Ad.vec and Ad.ΔRPH1+2 (FIG. 15D). Overexperssion of c-myc provided partial but significant protection so that colony formation was decreased by 46%, 44%, 53% and 46% upon infection with Ad.hPNPase^{old-35}, Ad.ΔRPH1, Ad.ΔRPH2 and Ad.ΔC-term, respectively. These findings suggest that downregulation of c-myc plays a prominent role in hPNPase^{old-35}-mediated growth inhibition.

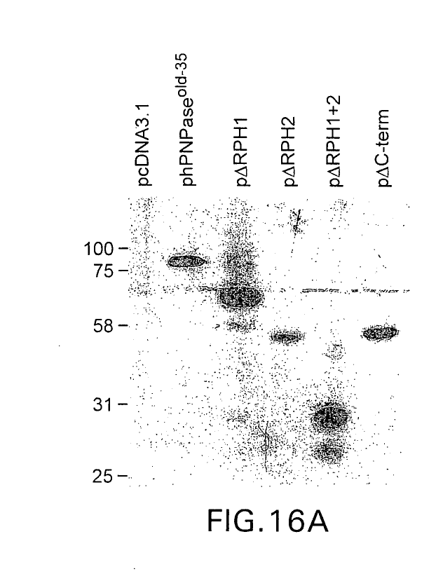

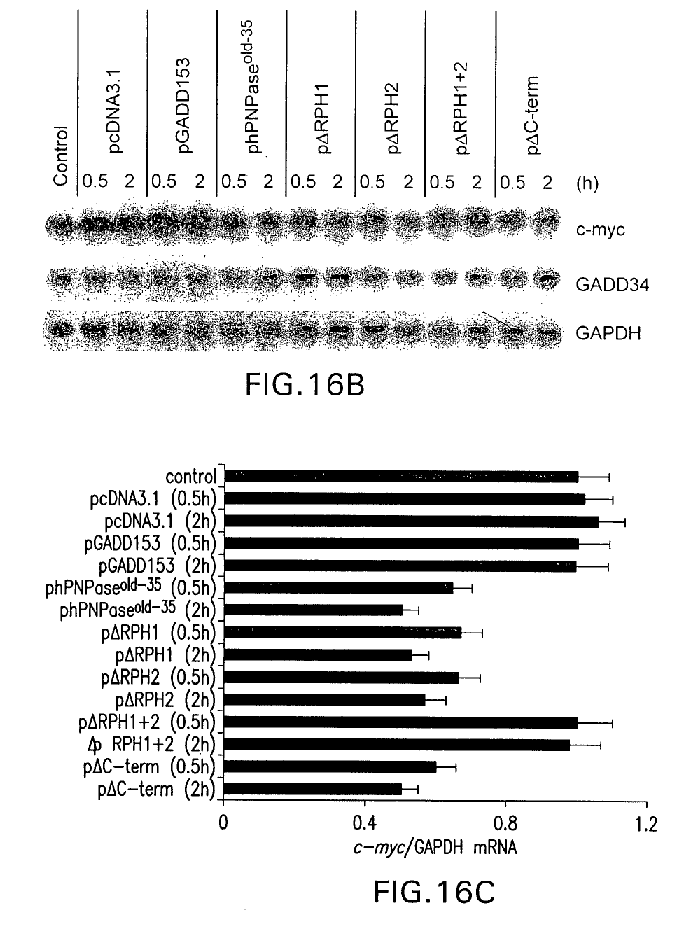

hPNPase^{old-35} is a 3′, 5′ exoribonuclease (75), so we investigated whether the downregulation of c-myc is a consequence of direct degradation of c-myc mRNA by hPNPase^{old-35}. hPNPase^{OLD-35} and its different deletion mutants were in vitro translated and the proteins were used for RNA degradation assays. As shown in FIG. 16A, the plasmid constructs phPNPase^{old-35}, pΔRPH1, pΔRPH2, pΔRPH1+2 and pΔC-term give rise to proteins of the expected molecular weight of 86-, 67-, 54-, 31- and 55-kDa, respectively, indicating the authenticity of the constructs. The in vitro translated proteins were incubated with total RNA for 0.5 and 2 h. The RNA was purified and the expression of c-myc, GADD34 and GAPDH mRNAs were analyzed by Northern blot analyses. Incubation with phPNPase^{old-35}, pΔRPH1, pΔRPH2 and pΔC-term resulted in significant degradation of c-myc mRNA (FIG. 16B, 16C). No degradation was observed upon incubation with the control plasmid pcDNA3.1, pΔRPH1+2 and an unrelated plasmid pGADD153 that expresses a transcription factor. The expressions of GADD34 and GAPDH mRNAs remained unchanged under all experimental conditions. These findings indicate that the presence of either RPH domain is necessary and sufficient to degrade c-myc mRNA and degradation is specific for c-myc mRNA.

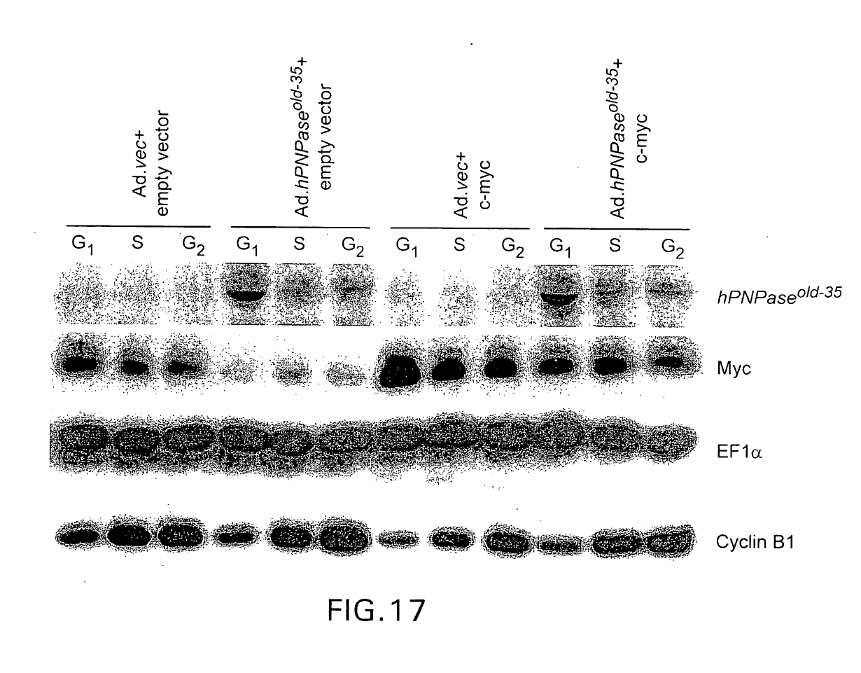

To establish a direct correlation between cell cycle arrest in the G₁ phase and c-myc downregulation by hPNPase^{OLD-35} HO-1 cells were sorted following Ad.hPNPase^{old-35} infection and the expressions of hPNPase^{OLD-35} and Myc were analyzed in the different phases of the cell cycle (FIG. 17). As a control for authenticity of the sorting procedure the expression of cyclin B1 was analyzed. Cyclin B1 starts being synthesized in late G₁ and its expression is maximum in G₂+M phase, which was confirmed by our cyclin B1 expression analysis in different phases of the cell cycle, indicating the effectiveness of the sorting procedure (FIG. 17, fourth panel). The equal expression of EF1-α in all the samples served as a loading control (FIG. 17, third panel). Although hPNPase^{OLD-35} protein could be detected in the cells in S and G₂+M phases, significantly higher levels of hPNPase^{OLD-35} were detected in cells in the G₁ phase of the cell cycle following Ad.hPNPase^{old-35} infection (FIG. 17, first panel, lanes 4 and 10 versus lanes 5, 6, 11 and 12). The level of hPNPase^{OLD-35} in different phases of the cell cycle inversely correlated with the level of Myc, which was markedly downregulated in cells in the G₁ phase (FIG. 17, second panel, lane 1 versus lane 4) and moderately downregulated in the cells in S and G₂+M phases (FIG. 17, second panel, lane 2, 3 versus lane 5, 6) following Ad.hPNPase^{old-35} infection. When Myc was overexpressed in cells, there was a slight reduction of Myc in cells in the G₁ phase upon Ad.hPNPase^{old-35} infection (FIG. 17, second panel, lane 7 versus lane 10) but this level of Myc was still markedly higher than that in Ad.hPNPase^{old-35}-infected G₁ phase cells without Myc overexpression (FIG. 17, second panel, lane 4 versus lane 10). The decrease of Myc in the Myc-overexpressed G₁ phase cells is probably because of downregulation of endogenous Myc. These findings indicate that hPNPase^{OLD-35} decreases endogenous but not exogenous Myc thereby explaining the potential protection against hPNPase^{OLD-35}-induced growth inhibition by overexpressing Myc. The c-myc expression plasmid does not contain the 3′ untranslated region (UTR) of the endogenous mRNA indicating that the sequence in the 3′ UTR of c-myc mRNA might contain a potential binding site for hPNPase^{OLD-35}.

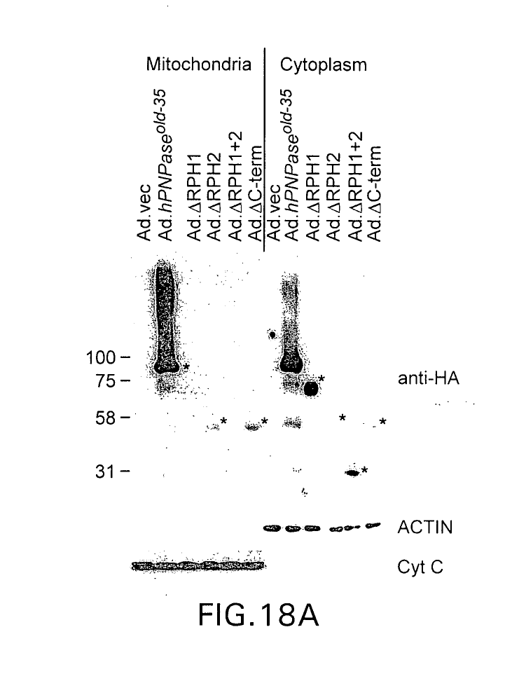

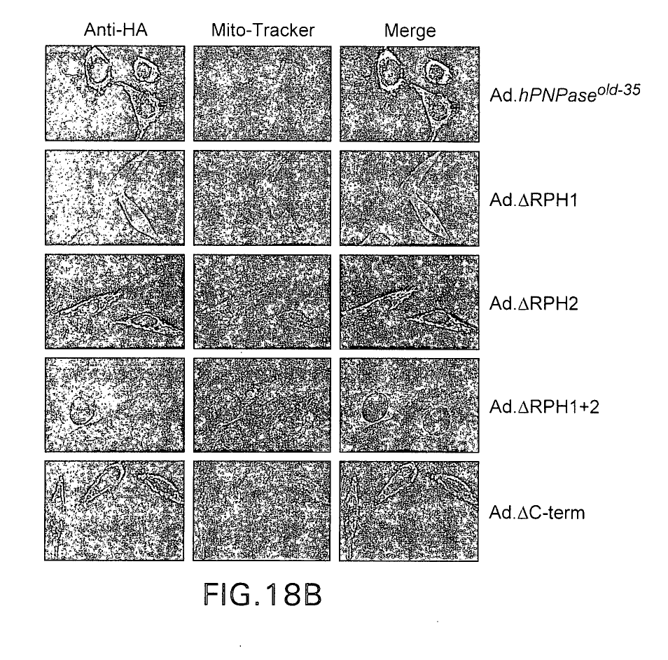

The presence of the mitochondrial localization signal indicates that hPNPase^{OLD-35} is a predominantly mitochondrial protein (79, 82). The question naturally arises as to how a mitochondrial protein might degrade a cytoplasmic mRNA, like c-myc. To address this question, cells infected with different Ad were fractionated into mitochondrial and cytoplasmic fractions and the expressions of the proteins were analyzed by Western blotting using anti-HA antibody. As a control for the quality of the purification, membranes were reprobed with anti-actin antibody (for cytoplasmic fraction) and anti-cytochrome C antibody (for mitochondria). hPNPase^{OLD-35}, ΔRPH2 and ΔC-term showed predominantly mitochondrial expression resulting from the presence of the mitochondrial localization signal in these constructs (FIG. 18A) while ΔRPH1 and ΔRPH1+2 was located predominantly in the cytoplasmic fraction (FIG. 18A) because of lack of the mitochondrial localization signal. However, high level expression of hPNPase^{OLD-35} and low level expression of ΔRPH2 and ΔC-term was also detected in the cytoplasmic fraction and very low level expression of ΔRPH1 was detected in the mitochondrial fraction. In the case of hPNPase^{OLD-35}, a smear was detected above the major band, which might result from incomplete denaturation of the homotrimer formed by hPNPase^{OLD-35}. These findings indicate that the proteins are localized both in cytoplasm and mitochondrial compartments and the bands that are detected are not a consequence of cross-contamination during purification, which is strongly supported by the observation that no cross staining for actin in mitochondria or for cytochrome c in cytoplasm was detected.

The fractionation results were confirmed by immunofluorescence studies using anti-HA antibody (green) and MitoTracker (red) to determine the localization of hPNPase^{OLD-35} and its deletion mutants (FIG. 18B). hPNPase^{OLD-35}, ΔRPH2 and ΔC-term showed a speckled expression pattern that co-localized with mitochondrial staining as evidenced by the presence of yellow color in the merged image. However, in addition to the yellow staining, there were also isolated green stainings observed in the merged images indicating the presence of hPNPase^{OLD-35}, ΔRPH2 and ΔC-term in cytoplasmic compartments. ΔRPH1 and ΔRPH1+2 showed a diffuse expression pattern throughout the cytoplasm that did not co-localize with the mitochondrial staining indicating the presence of ΔRPH 1 and ΔRPH 1+2 in the cytoplasm.

7.4 Discussion

In this study, the importance of the RPH domains in mediating the characteristic phenotypic changes induced by hPNPase^{old-35} is firmly established. We observed that presence of at least one RPH domain is required for the functional activity of the protein and either of the domains is as potent as the full-length molecule. This contrasts with bacterial PNPase in which mutation in the key residues in either of the RPH domains inhibits catalytic activity (70). However, our result is comparable to chloroplast PNPase in which the first RPH domain alone (comparable to our ΔRPH2 construct) is highly active enzymatically (91). Although the second RPH domain (our ΔRPH1) of chloroplast has low RNA degradation activity of non-polyadenylated RNA it has high activity for polyadenylated RNA (91) which explains the efficiency of the ΔRPH1 construct in degrading human polyadenylated mRNAs. The RPH domains themselves can bind to RNA and the PNPase domain is also involved in RNA binding which explains the preservation of the RNA degradation activity of the AC-term construct that lacks the KH and S1 RNA binding domains. Current studies are determining the effects of mutation in specific important and evolutionary conserved amino acid residues of hPNPase^{old-35}, especially those surrounding the tungsten binding region implicated in the enzymatic activity of the bacterial protein (70).

A unique and potentially significant finding is that hPNPase^{OLD-35} displays specific degradation activity for c-myc mRNA. The 3′ stem-loop structure of a RNA species determines the enzymatic activity of PNPases (91). It is of significant interest to see whether there exists a difference between the 3′ secondary structure of c-myc and other mRNAs, such as GAPDH and GADD34, that facilitates its degradation by hPNPase^{old-35}. PNPases function as a homotrimer (53) and our findings suggest that hPNPase^{OLD-35} also multimerizes. However, the deletion mutants, that do not multimerize, still retain their specific mRNA degradation activity. We can rule out the involvement of a c-myc specific RNA binding protein regulating the activity of hPNPase^{OLD-35}, since our in vitro RNA degradation assays, which contained a single protein, could effectively achieve RNA degradation. Elucidation and comparison of three-dimensional models of c-myc and other mRNAs might provide insights into the specific RNA degradation activity of hPNPase^{old-35}.

Structure analysis and localization studies reveal that hPNPase^{OLD-35} is a predominantly mitochondrial protein (79, 82), thereby provoking the obvious question as to how hPNPase^{OLD-35} might degrade a cytoplasmic target mRNA, such as c-myc. By fractionation and immunofluorescence analyses, we demonstrate that although the primary site of localization of hPNPase OLD-3 is the mitochondria, a considerable amount of the protein is also present in the cytoplasmic fraction of the cell. Similarly, ΔRPH2 and ΔC-term, that retain the mitochondrial localization signal, are also mainly localized in mitochonidria, although low but detectable levels are still found in the cytoplasm. ΔRPH1 and ΔRPH1+2, lacking the mitochondrial localization signal, were located in the cytoplasm although a very low level of ΔRPH1 was also evident in the mitochondria. This is not because of cross-contamination which was confirmed by mutually exclusive expression of cytochrome c and actin in mitochondria and cytoplasmic fractions, respectively. These findings suggest the existence of a potential cytoplasmic-mitochondrial shuttling of the hPNPase^{OLD-35} protein that might be regulated by chaperone proteins. Further studies designed to identify and define interacting partners of hPNPase^{OLD-35} will help address this important question.

Adenovirus-mediated delivery usually results in robust transgene expression that raises the important question of whether the senescence-inducing effect of Ad.hPNPase^{old-35} is a genuine physiological phenomenon. We have observed that there is a comparable level of hPNPase^{OLD-35} protein upon IFN-β treatment and with Ad.hPNPase^{old-35} infection suggesting that the senescent phenotype observed with hPNPase^{OLD-35} is indeed a physiologically relevant event. Bacterial PNPase autocontrols its expression post-transcriptionally by degrading its own mRNA (71). Although not yet confirmed for hPNPase^{old-35}, this post-transcriptional control might explain the restricted expression level of hPNPase^{OLD-35} even with adenovirus-mediated delivery of this enzyme.

CDKIs, especially p16^{INK4A} and p21^{CIP1/WAF-1/MDA-6} are intimately involved in the process of cellular senescence (52, 87). hPNPase^{old-35}-induced senescence is associated with an increase in p27^{KIP1} and a decrease in p21^{CIP1/WAF-1/MDA-6}, a phenomenon that is observed in two other models of senescence-like growth arrest, one resulting from iron chelation in hepatocytes, and the other a consequence of inhibition of the phosphoinositide-3-kinase pathway in mouse embryo fibroblasts (57, 92). The involvement of p16 ^{INK4A} is ruled out as an essential component of this process because HO-1 cells do not express the p16^{INK4A} protein. The increase in p27^{KIP1} is most likely secondary to the decrease in c-myc that controls p27^{KIP1} expression at multiple levels, including repression of transcription and facilitation of ubiquitination and subsequent degradation (77, 78, 90). Myc plays an important role in controlling cell cycle (65). It promotes entry into the S phase and shortens the G₁ phase. We observed that downreguation of c-myc by hPNPase^{old-35} resulted in a corresponding increase in Mad1 and this particular shift from MYC-MAX transcriptional activator to MAD1-MAX transcriptional repressor might be important for mediating the plethora of effects promulgated by hPNPase^{old-35}. However, since overexpression of Myc could not provide complete protection against hPNPase^{old-35}-mediated growth inhibition, additional targets of hPNPaseold-35 are likely to exist that might be involved in mediating its effects. While the effect of hPNPase^{old-35} on c-myc is direct, its effect on MAD1 is probably indirect via additional targets, downregulation of which might lead to derepression of MAD1 expression.

In summary, we now confirm the importance of the RPH domains in hPNPase in inducing senescence, an event that is unique in its molecular mechanism and is critical for regulating organismal homeostasis. These studies provide unique perspectives on the molecular mechanism of senescence and the structure-function relationship of the hPNPase^{OLD-35} protein. Crystallization of the hPNPase^{old-35} protein and comparison of its crystal structure to that of the PNPase proteins of other species will directly facilitate identification of important residues mediating catalytic and senescence-inducing activity. Of added significance, the development of an animal model conditionally overexpressing hPNPase^{old-35} will provide valuable insights into the involvement of hPNPase^{old-35} in in vivo senescence.

### 8. EXAMPLE

### Defining the Mechanism by which IFN-β Dowregulates c-myc Expression in Human Melanoma Cells: Pivotal Role for Human Polynucleotide Phosphorylase (hPNPase^{old-35})

8.1 Materials and Methods

Cell lines and cell viability assays: FM516-SV (referred to as FM516) normal immortal human melanocyte, WM35 early radial growth phase (RGP) primary human melanomas, HO-1 and MeWo metastatic melanomas, 2fTGH human fibrosarcoma and its derivates U1A, U3A, U4A and U5A, HeLa human cervical carcinoma and 293 adenovirus transformed human embryonic kidney (HEK 293) cells were cultured in Dulbecco's modified Eagle's medium containing 10% fetal bovine serum and penicillin (100 U/ml) and streptomycin (100 μg/ml). 2fTGH cells are wild type in IFN signaling while its derivates U1A, U3A, U4A and U5A have defects in IFN signaling that could be complemented by expression of TYK2, STAT1, JAK1 or IFNAR2, respectively (112, 113). Cell growth and viable cell numbers were monitored by 3-(4,5-dimethylthiazol-2-yl)-2,5-diphenyl tetrazolium bromide (MTT) staining as described (150).

Generation of lentivirus expressing siRNA for hPNPase^{old-35}: Using the software siRNA Target Finder (Ambion, Austin, Tex.) four potential siRNAs for hPNPase^{old-35} were designed and the siRNAs were constructed by in vitro transcription using the Silencer siRNA construction kit (Ambion) according to the manufacturer's protocol. These siRNAs were transfected into HeLa cells using Lipofectamine 2000 (Invitrogen, Carlsbad, Calif.) according to the manufacturer's protocol and the next day the cells were treated with 1000 U/ml IFN-β for 24 h. The expression of hPNPase^{OLD-35} in the lysates of these cells was analyzed by Western blot analysis. The siRNA demonstrating the maximum inhibition of hPNPase^{OLD-35} induction by IFN-β was selected for construction of the lentivirus. The hPNPase^{old-35} and control siRNA sequences were 5′ AACAAAACCTTCCCCTTCCCA 3′ (SEQ ID NO:40) and 5′ AAGGGTCGTCTATAGGGATCGAT 3′ (SEQ ID NO:41), respectively. Lentiviruses expressing either control siRNA or hPNPase^{old-35} siRNA were constructed using BLOCK-iT Lentiviral RNAi Expression System (Invitrogen) according to the manufacturer's protocol. The siRNA was first ligated into BLOCK-iT U6 RNAi Entry Vector that drives expression of the siRNA under control of the human U6 promoter. The siRNA expression cassette was transferred to pLenti6 BLOCK-iT-DEST lentiviral vector by the LR recombination reaction. The resultant construct was transfected into HEK293FT cells with Lipofectamine 2000 along with ViraPower lentiviral packaging mix that expresses the proteins required for lentivirus replication. The lentivirus was amplified and titered by standard plaque assay.

Generation of stable cell clones: Stable clones of HO-1 cells expressing either control siRNA or hPNPase^{old-35} siRNA were generated by transducing the cells with lentiviruses expressing the corresponding siRNA and selecting clones for 2 weeks using 4 μg/ml blasticidin. Stable HO-1 clones expressing c-myc were generated by transfecting HO-1 cells with a c-myc expression vector and selecting the cells for 2 weeks with 100 μg/ml hygromycin. Transfecting the cells with empty vector and selecting with hygromycin generated corresponding control clones.

Cell Cycle Analysis: Cells were harvested, washed in PBS and fixed overnight at −20° C. in 70% ethanol. The cells were treated with RNase A (1 mg/ml) at 37° C. for 30 min and then with propidium iodide (50 μg/ml). Cell cycle was analyzed using a FACScan flow cytometer and data were analyzed using CellQuest software (Becton Dickinson, San Jose, Calif.).

Colony formation assays: One-thousand cells were plated in 6-cm dishes and then treated with different doses of IFN-β for 2 weeks at which point the colonies were fixed, stained with Giemsa and colonies≧50 cells were counted.

Transfection of siRNA: Cells (5×10⁵) were plated in a 6 cm dish and the next day were transfected with 25 nM of either control siRNA or c-myc siRNA (Ambion; catalogue# 4250) using Lipofectamine 2000 (Invitrogen) according to the Manufacturer's protocol. After 24 h, the cells were trypsinized and seeded into 96-well plates for cell viability assays and 6-cm dishes for colony formation assays and cell cycle analyses as described above.

RNA Isolation and Northern Blot Analysis: Total RNA was extracted from cells using Qiagen RNeasy mini kit (Qiagen) according to the manufacturer's protocol and Northern blotting was performed as described (102). The cDNA probes used were a 400-bp fragment from human c-myc, a 500-bp fragment from hPNPase^{old-35} and full-length human GAPDH. For analysis of half-life of c-myc mRNA, cells were either untreated or treated with IFN-β (1000 U/ml) for 24 h and then treated with Actinomycin D (5 μg/ml) for 0.5, 1, 2, 4 and 8 h following which the cells were harvested for total RNA extraction and Northern blot analysis.

Western Blot Analysis: Western blotting was performed as previously described (102). Briefly, cells were harvested in RIPA buffer containing protease inhibitor cocktail (Roche, Mannheim, Germany), 1 mM Na₃VO₄ and 50 mM NaF and centrifuged at 12,000 rpm for 10 min at 4° C. The supernatant was used as total cell lysate. Thirty μg of total cell lysate were used for SDS-PAGE and transferred to a nitrocellulose membrane. The primary antibodies included: Myc (1:200; mouse monoclonal; Santa Cruz biotechnology, Santa Cruz, Calif.), hPNPase^{OLD-35} (1:10000; chicken polyclonal), MDA-5 (1:5000; rabbit polyclonal) and EF1α (1:1000; mouse monoclonal; Upstate Biotechnology, Waltham, Mass.).

Statistical analysis: Statistical analysis was performed using one-way analysis of variance (ANOVA), followed by Fisher's protected least significant difference analysis.

8.2 Results

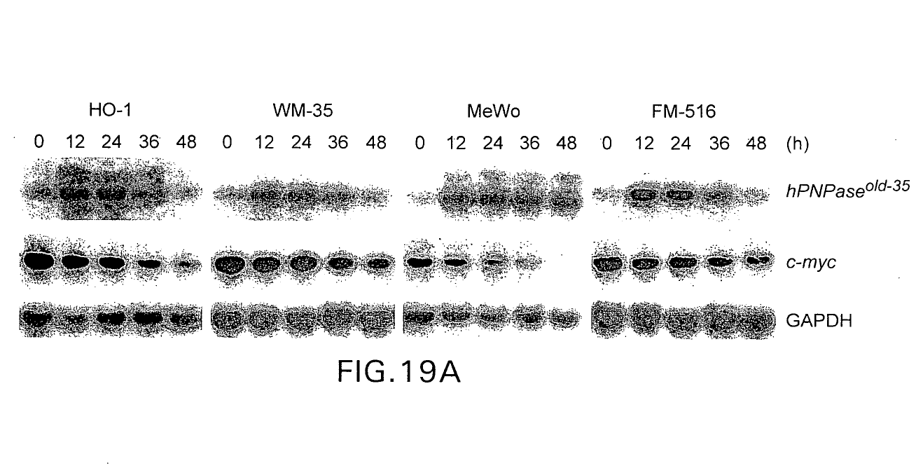

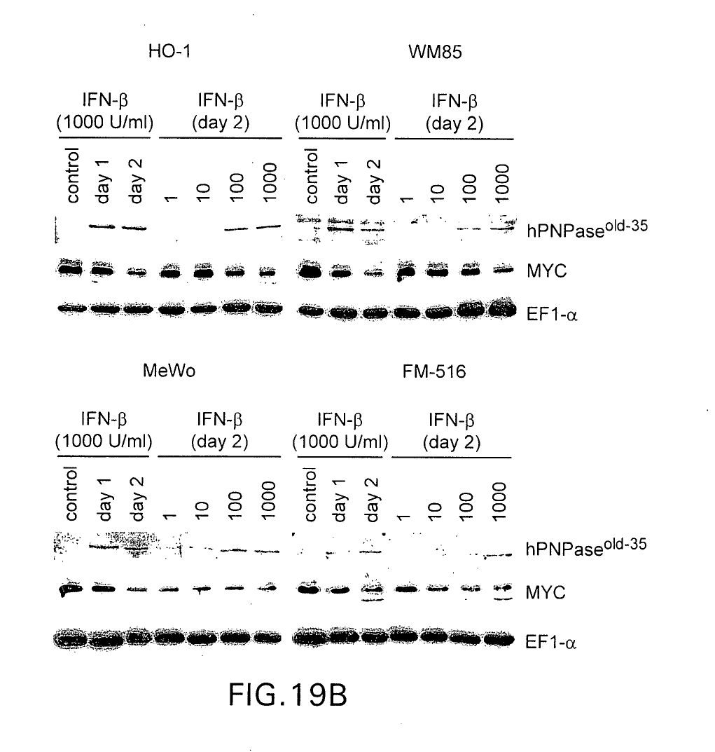

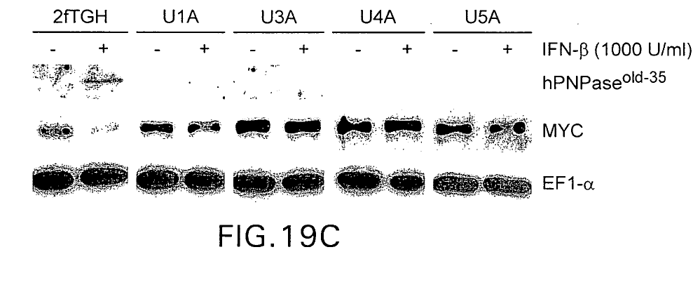

Regulation of hPNPase^{old-35} and c-myc mRNA expression by IFN-β: To define a potential correlation between the expression regulation of hPNPase^{old-35} and c-myc by IFN-β, HO-1, WM35 and MeWo human melanoma cells and SV40 T/t Ag-immortalized human melanocytes (FM-516-SV, here forth indicated as FM-516) were treated with 1000 U/ml of IFN-β for different times ranging from 12 to 48 h and the expression of hPNPase^{old-35} and c-myc mRNA was determined by Northern blot analysis (FIG. 19A). Under basal condition, there was little to barely detectable hPNPase^{old-35} mRNA expression in the different cell types. Upon IFN-β-treatment a marked increase in hPNPase^{old-35} mRNA expression was detected 12 h post-treatment. In HO-1, WM-35 and FM-516 cells hPNPase^{old-35} mRNA expression gradually decreased with time and by 48 h post-IFN-β-treatment returned to the basal level. However, in MeWo cells hPNPase^{old-35} mRNA expression persisted even 48 h after IFN-β-treatment. While hPNPase^{old-35} mRNA expression was increased by IFN-β, c-myc mRNA expression decreased with the same treatment and there was a temporal correlation in the expression regulation of these two mRNAs by IFN-β. A significant time-dependent IFN-β-mediated decrease in c-myc mRNA expression was also evident in all four cell lines. In MeWo cells, with persistence of hPNPaseold-35 mRNA expression, c-myc mRNA expression disappeared completely at 48 h post IFN-β-treatment.

The mRNA expression results were confirmned on a protein level by Western blot analysis (FIG. 19B). Under basal condition, hPNPase^{OLD-35} protein was undetectable in all four cell lines. With IFN-β-treatment, hPNPase^{OLD-35} protein expression was markedly induced and persisted even 2 days after treatment. With the exception of MeWo cells, the corresponding mRNA levels decreased at 48 h in HO-1, WM-35 and FM-516 cells. The Myc protein levels also showed a temporal decrease following IFN-β-treatment.

A direct correlation in IFN-β-induced dose-dependent changes in hPNPase^{OLD-35} and Myc proteins was also evident. In HO-1 and WM-35 cells, hPNPase^{OLD-35} induction and Myc downregulation were detected with 100 and 1000 U/ml of IFN-β, but not with 1 or 10 U/ml (FIG. 19B). In MeWo and FM-516 cells, changes in protein levels could be detected with as little as 1 U/ml or 10 U/ml of IFN-β, respectively. In IFN-β-treated FM-516 cells in addition to the Myc band, a faster migrating band was detected which might represent a degradation product of the Myc protein. These findings confirm that the concentration of IFN-β required to upregulate hPNPase^{OLD-35} is also required to downregulate Myc protein indicating a potential cooperative regulation in the expression of these two genes.

The regulation of expression of hPNPase^{OLD-35} and Myc by IFN-β was confirmed in 2fTGH human fibrosarcoma cells and in its four variants. U1A (Tyk2-), U3A (STAT1-), U4A (JAK1-) and U5A (IFNAR2-), that have mutations in different molecules involved in the IFN-signaling pathway (112, 113). As shown in FIG. 19C, the upregulation of hPNPase^{OLD-35} and downregulation of Myc by IFN-β were observed only in parental 2fTGH cells but not in its mutant clones, which are non-responsive to type I IFN.

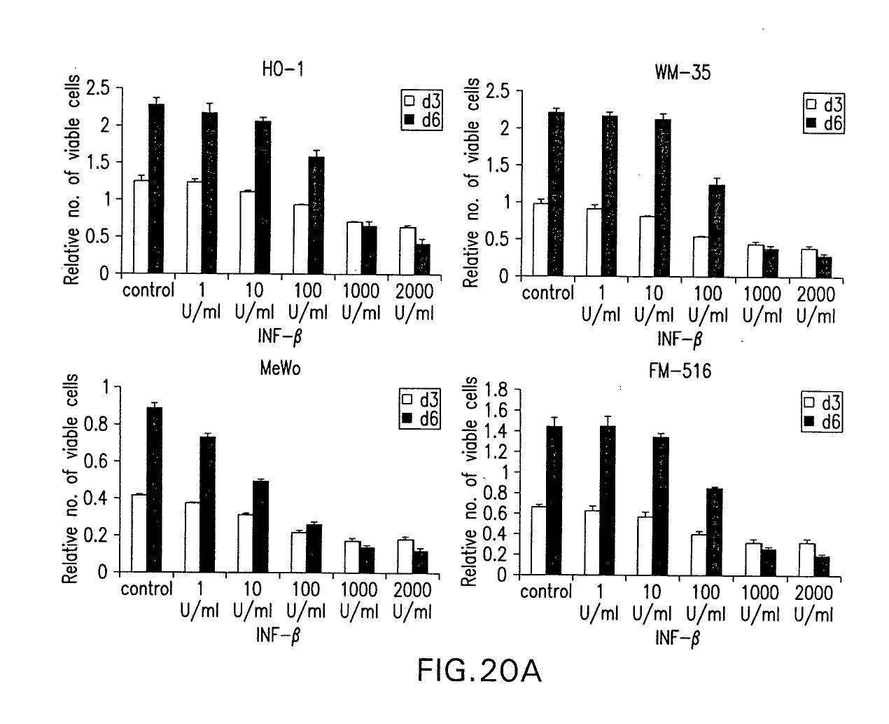

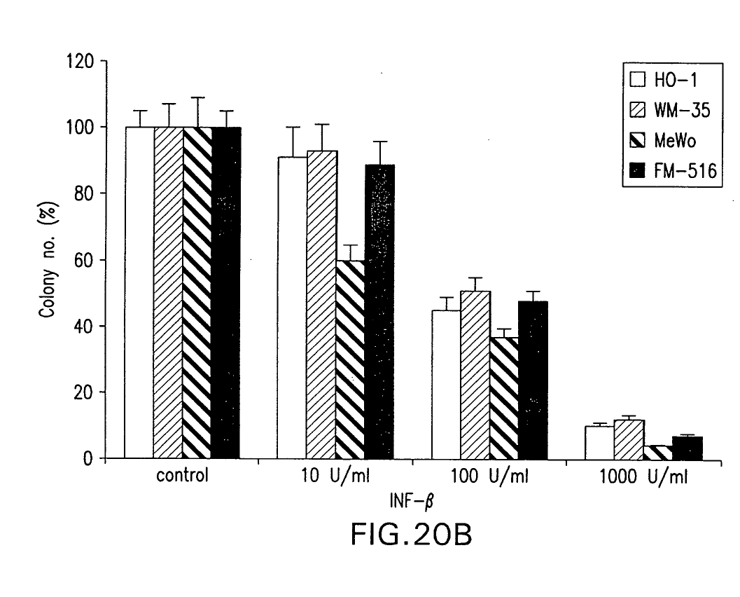

IFN-β its growth of melanoma cells and melanocytes: The effect of IFN-β on the growth of HO-1, WM35, MeWo and FM-516 cells were analyzed by standard MTT cell survival assays (FIG. 20A). Cells were treated with 1, 10, 100, 1000 and 2000 U/ml of IFN-β for up to 6 days. HO-1, WM-35 and FM-516 cells did not respond to 1 or 10 U/ml of IFN-β. With 100 U/ml, there was a significant inhibition in cell growth and with 1000 U/ml and 2000 U/ml, there was ˜90% inhibition in growth 6 days after IFN-β-treatment. The growth of MeWo cells was significantly inhibited even with 1 U/ml of IFN-β, which became marked with 100 or more U/ml of IFN-β. These studies document a direct correlation between gene expression changes and the levels of IFN-β required to evoke growth inhibition in specific target cells. This finding is particularly relevant in the case of MeWo cells in which corresponding changes could be observed even with 1 U/ml of IFN-β.

The results obtained using cell viability assays were confirmed by colony formation assays (FIG. 20B). In HO-1, WM-35 and FM-516 cells colony formation was significantly inhibited with 100 U/ml of IFN-D and with 1000 U/ml, the colony formation was inhibited by >90%. In the case of MeWo cells, 10 U/ml of IFN-β significantly inhibited colony formation and with 1000 U/ml of IFN-β colony formation was inhibited by >95%.

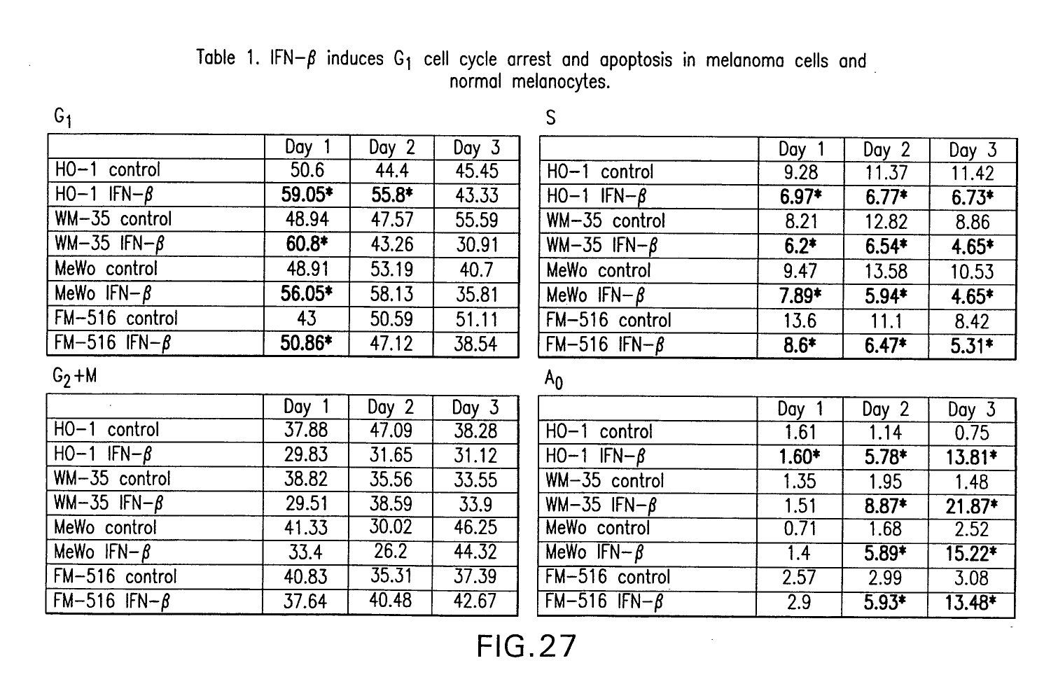

Cell cycle analysis was performed to characterize growth inhibition. IFN-β treatment (1000 U/ml) in HO-1, WM-35, MeWo and FM-516 cells resulted in initial (day 1) cell cycle arrest in the G₁ phase of the cell cycle with a concomitant decrease in the DNA synthesis phase as substantiated by the reduction in S phase (FIG. 27-Table 1). With longer exposure to IFN-β the cells gradually became apoptotic as evidenced by a steady increase in the sub-G₁ (A₀) cell population.

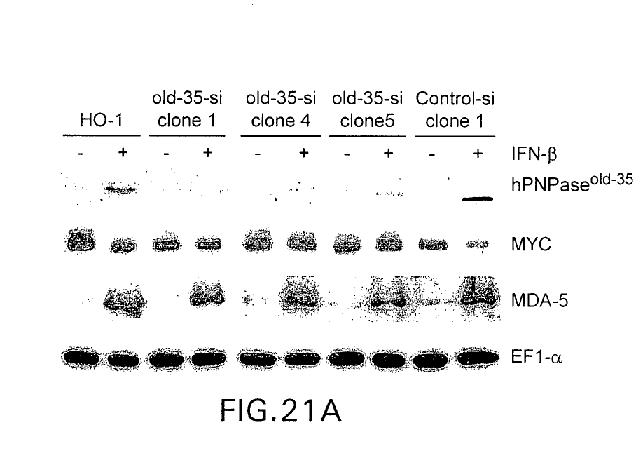

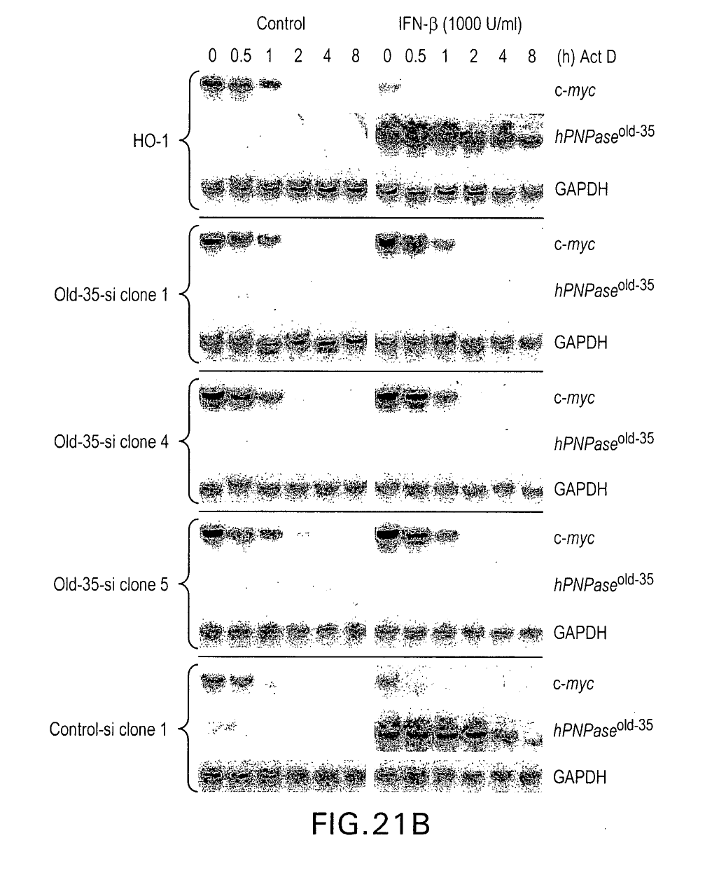

hPNPase^{OLD-35} regulates IFN-β-mediated downregulation of Myc: Since hPNPase^{OLD-35} is a 3′, 5′ exoribonuclease and one of its substrates is c-myc mRNA we tested whether hPNPase^{OLD-35}, induced by IFN-β, promotes Myc downregulation. We have identified siRNA active in downregulating hPNPase^{old-35} and created a lentivirus expressing this siRNA. Stable clones in an HO-1 background expressing either control siRNA or hPNPase^{old-35} siRNA were generated by selection with blasticidin. As shown in FIG. 21A, three clones, clone 1, 4 and 5, which express hPNPase^{old-35}-siRNA, were identified that significantly inhibited IFN-β-induction of hPNPase^{OLD-35}, with clone 1 being the most efficient producing almost 100% inhibition in hPNPase^{OLD-35} induction. The clone expressing control siRNA retained its ability to induce hPNPase^{OLD-35} following IFN-β treatment. Remarkably, while the parental HO-1 cells and control-siRNA expressing clone could downregulate Myc in response to IFN-β treatment, all three hPNPase^{old-35} siRNA expressing clones lost this ability. However, these clones retained their ability to respond to IFN-β as evidenced by the induction of another IFN-inducible gene mda-5 (114). These findings indicate that hPNPase^{OLD-35} specifically mediates downregulation of Myc but not the modulation of other genes by IFN-β. The observation of similar responses in multiple clones rules out the possibility that the observed effects are simply a consequence of clonal variability in response to IFN-β.

The half-life of c-myc mRNA with or without IFN-β treatment was analyzed in HO-1 cells and control siRNA and hPNPase^{old-35} siRNA expressing clones (FIG. 21B). The cells were treated with IFN-β (1000 U/ml) for 24 h and then exposed to Actinomycin D (Act D; 5 μg/ml) for 0.5 to 8 h (FIG. 21B). In the untreated cells, the half-life of c-myc mRNA was ˜1 h. In HO-1 cells and control siRNA expressing clones, IFN-β treatment resulted in significant downregulation of c-myc mRNA so that by 0.5 h of Act D treatment no c-myc mRNA could be detected in these cells. This downregulation correlated with upregulation of hPNPase^{old-35} mRNA that had a half-life of ˜4 h. In contrast, IFN-β treatment did not induce hPNPase^{old-35} mRNA expression in hPNPase^{old-35}-siRNA expressing clones and the half-life of c-myc mRNA remained unchanged when compared to control untreated cells (FIG. 21B). These findings indicate that under basal condition hPNPase^{old-35} is not expressed and therefore it does not affect the turnover of c-myc mRNA. However, upon IFN-β treatment this enzyme is induced and it degrades c-myc mRNA.

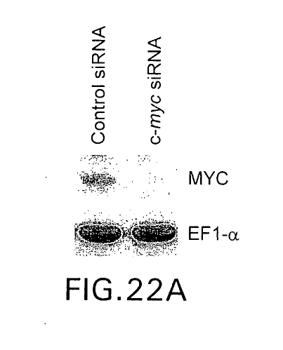

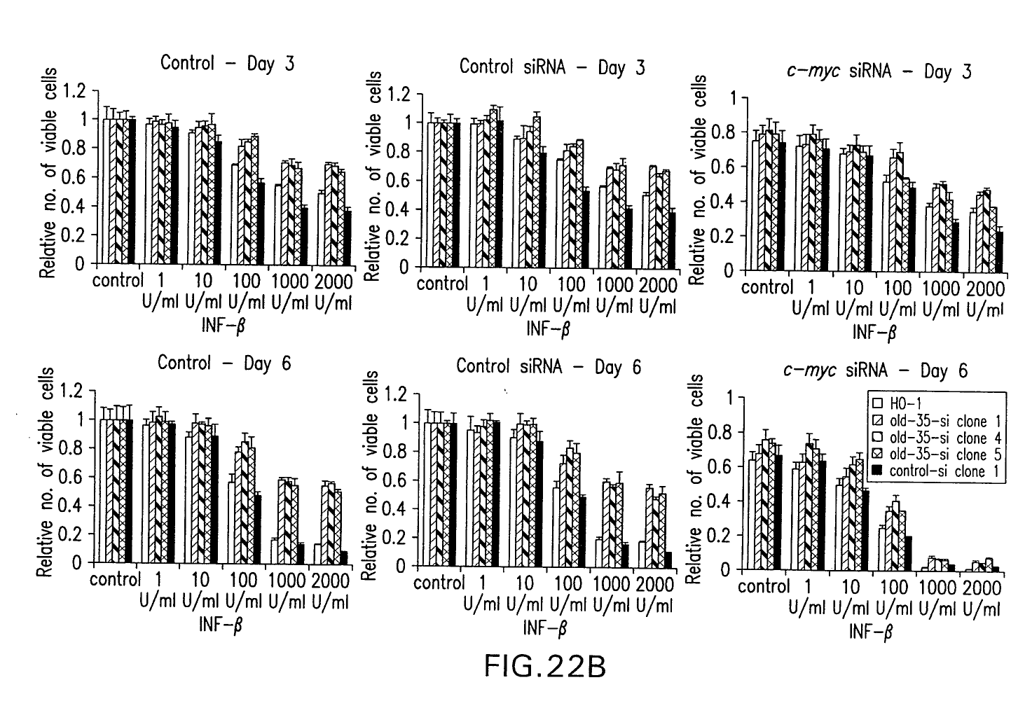

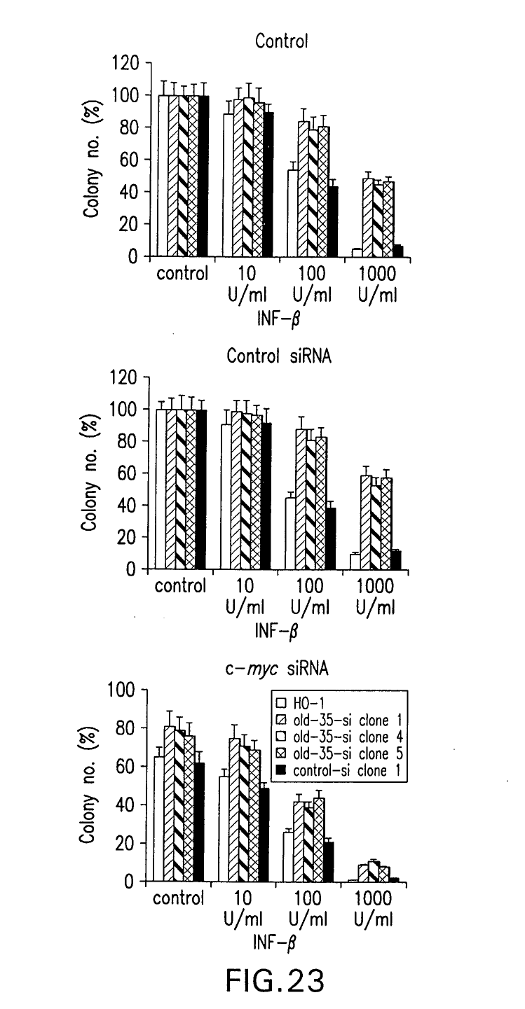

Resistance of hPNPase^{old-35}-siRNA clones to IFN-β-mediated growth inhibition: Overexpression of hPNPase^{old-35} induces growth inhibition and apoptosis in melanoma cells and c-myc is a positive regulator of cell growth, allowing cells to traverse the G₁ phase of the cell cycle. Based on these considerations, we tested whether the lack of these two events in hPNPase^{old-35}-siRNA expressing clones would render them resistant to IFN-β-mediated growth inhibition. As shown in FIG. 22B, while the parental HO-1 cells and control siRNA expressing clones were sensitive to IFN-β-treatment, as monitored by standard MTT assays, the hPNPase^{old-35}-siRNA expressing clones showed significant resistance to IFN-β, which became more pronounced after 6 days of IFN-β treatment. These findings were confirmed by colony formation assays, which also demonstrated significant resistance of hPNPase^{old-35}-siRNA expressing clones to IFN-β-induced inhibition of colony formation (FIG. 23).

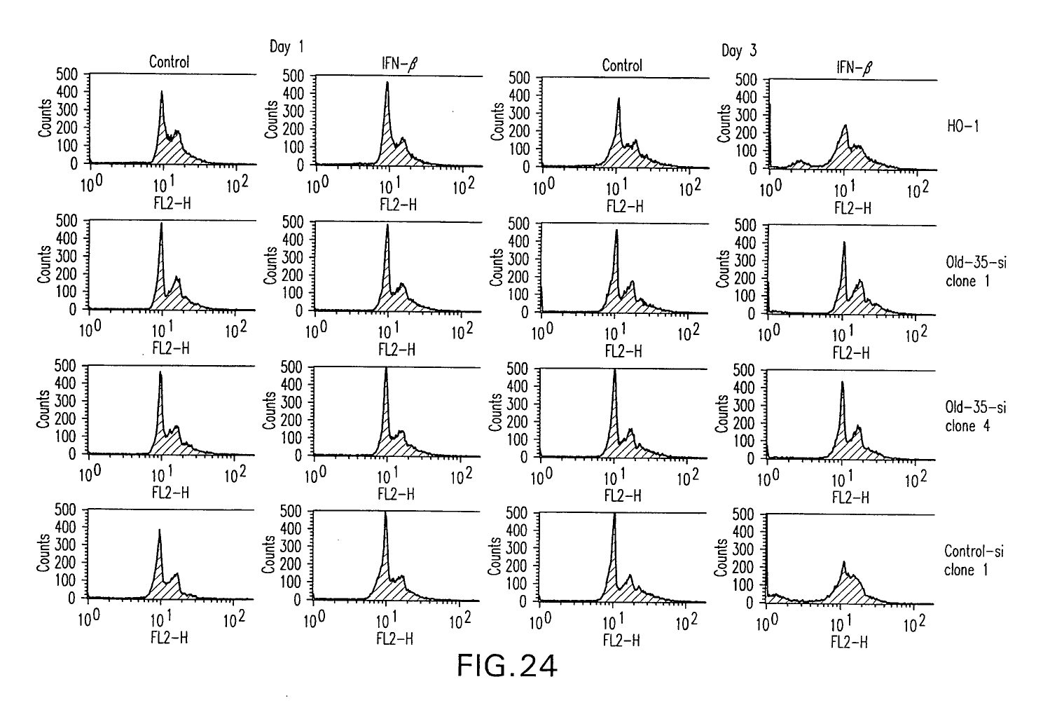

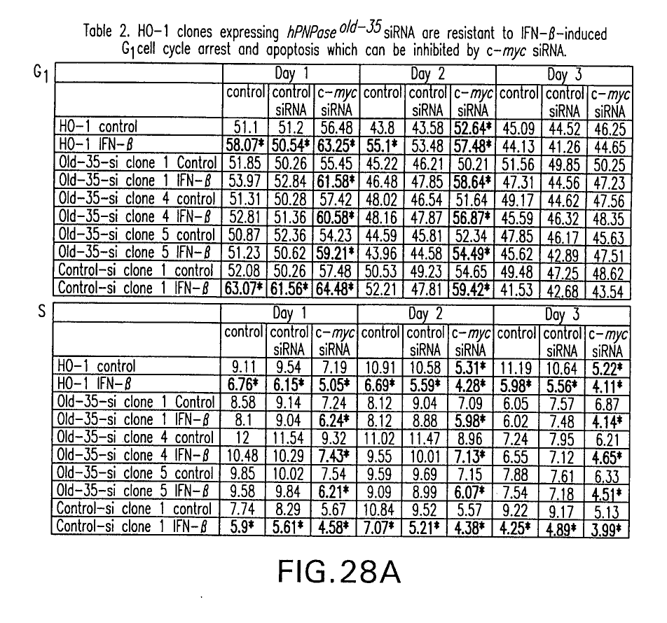

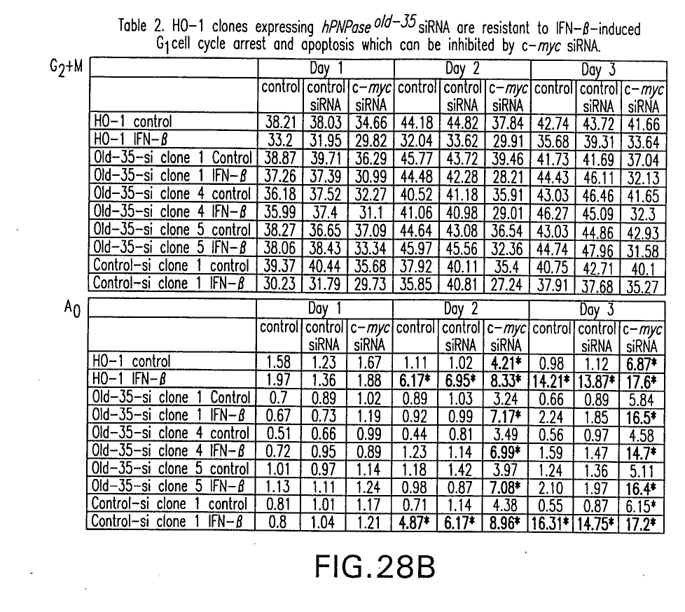

The results obtained from cell survival and colony formation assays were confirmed by cell cycle analysis using flow cytometry. As shown in FIG. 24 and FIG. 28-Table 2, the parental HO-1 cells and control siRNA expressing clones showed an initial G₁ arrest and eventually cells underwent apoptosis. However, hPNPase^{old-35}-siRNA expressing clones showed remarkable resistance to growth inhibition by IFN-β with no statistically significant increase in the G₁ phase or the number of A₀ cells. In these contexts, blocking hPNPase^{old-35} expression prevents cell cycle arrest and apoptosis induced by IFN-β.

To confirm that the mechanism underlying the resistance of hPNPase^{old-35}-siRNA expressing clones to IFN-β is mediated by their inability to downregulate c-myc, HO-1 cells and control siRNA and hPNPase^{old-35}-siRNA expressing clones were transfected with either control or c-myc siRNA and treated with IFN-β and cell viability, colony formation and cell cycle analyses were performed. Transfection of c-myc siRNA resulted in marked downregulation of Myc protein (FIG. 224A) indicating the authenticity of its function. Cell viability and colony formation ability was similar between control untransfected cells and control. siRNA-transfected cells (FIGS. 22B and 23) with hPNPase^{old-35}-siRNA expressing clones showing resistance to IFN-β and HO-1 cells and control siRNA expressing clones showing sensitivity to IFN-β. Transfection of c-myc siRNA alone reduced cell viability and colony formation ability of all the cell lines and together with IFN-β markedly inhibited cell viability and colony formation ability in all of the cell lines, including hPNPase^{old-35}-siRNA expressing clones (FIGS. 22B and 23). Cell cycle analysis also revealed that transfection of c-myc siRNA rendered hPNPase^{old-35}-siRNA expressing clones susceptible to IFN-β mediated cell cycle arrest and apoptosis (FIG. 28-Table 2). In total, these findings indicate that inhibition of c-myc downregulation in hPNPase^{old-35}-siRNA expressing clones confers their resistance to growth inhibition by IFN-β.

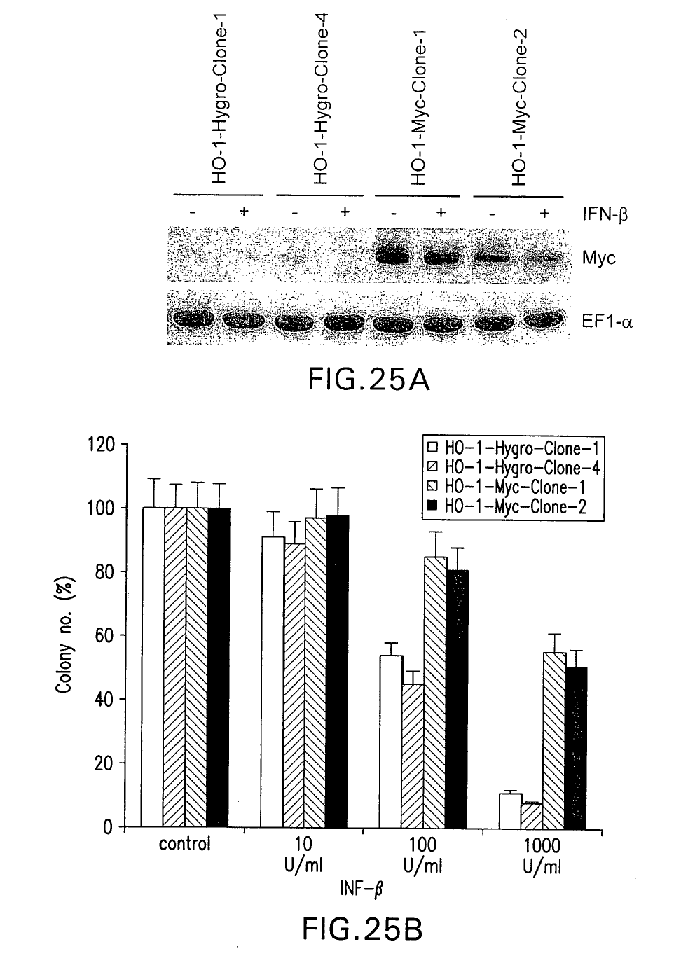

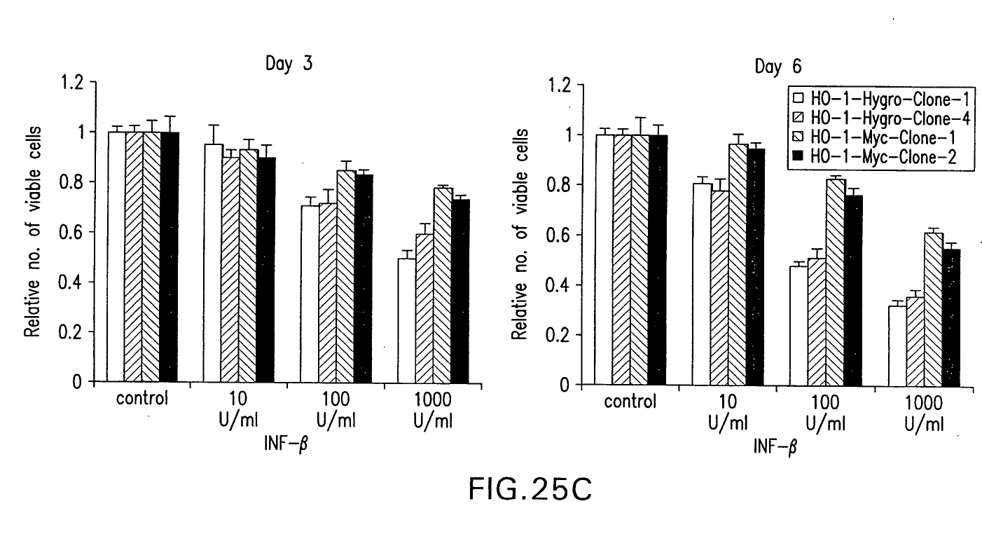

Resistance of c-myc overexpressing clones to IFN-β-mediated growth inhibition: We next evaluated the involvement of c-myc downregulation in IFN-β-mediated growth inhibition. For this purpose, stable Myc overexpressing HO-1 clones (HO-1-Myc) were developed by transfection with a c-myc expression vector and selection with hygromycin. Control hygromycin-resistant clones (HO-1-Hygro) were similarly generated. FIG. 25A provides data from two representative Myc-overexpressing HO-1 clones. IFN-β treatment for 3 days resulted in marked downregulation of endogenous Myc protein in HO-1-Hygro clones (FIG. 25A). However, the exogenous Myc protein in HO-1-Myc clones was not significantly downregulated by IFN-β. The c-myc expression construct contains only the open reading frame and not the 3′ or 5, untranslated regions (UTR) of the cDNA. The inability of IFN-β to downregulate exogenous c-myc indicates that the 3′-UTR of the endogenous c-myc sequence might confer its sensitivity to hPNPase since hPNPase^{OLD-35} is a 3′, 5′ exoribonuclease. The growth of the HO-1-Hygro clones (clones 1 and 4) was significantly inhibited by IFN-β (1000 U/ml) treatment as documented by cell viability assays (FIG. 7C). HO-1-Myc clones overexpressing Myc provided partial but significant protection from IFN-β-mediated growth inhibition (FIG. 25C). These findings were also confirmed by colony formation assays (FIG. 25B). HO-1-Myc clones, but not HO-1-Hygro clones, showed resistance to inhibition of colony formation by IFN-β. These results implicate IFN-β modulation of c-myc as an important factor associated with IFN-β-induced growth suppression in HO-1 cells.

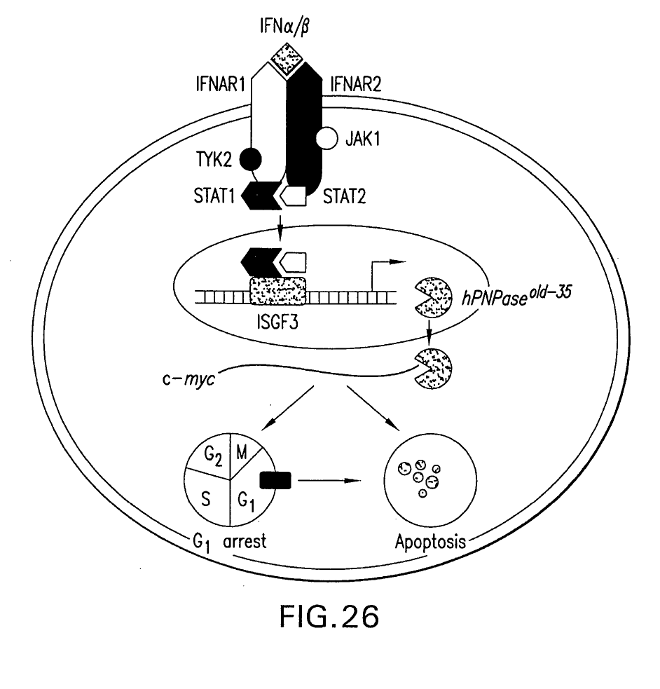

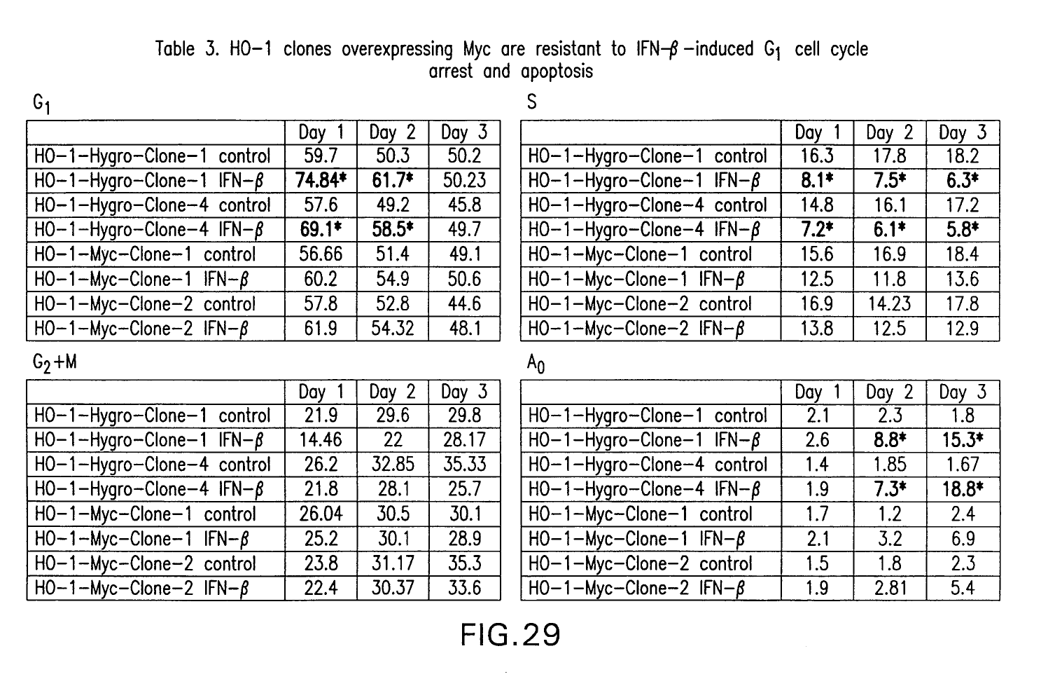

These interesting findings were corroborated by cell cycle analysis. Treatment with IFN-β induced an initial G₁ arrest and eventually apoptosis in HO-1-Hygro clones (FIG. 29-Table 3). The HO-1-Myc clones showed a slight increase in the percentage of G₁ phase and apoptotic cells following IFN-β treatment, which was not statistically significant. These findings indicate that both upregulation of hPNPase^{old-35} and downregulation of c-myc are central events in mediating the ability of IFN-β to inhibit growth in human melanoma cells (FIG. 26).

8.3 Discussion

Microarray studies have revealed a plethora of genes that are modulated by IFN treatment (115) (http://bioinfo.cnio.es/data/oncochip/). IFNs can directly affect gene expression by ISRE and GAS sequences in the promoters of target genes (97). In addition, IFNs can also affect gene expression by their ability to induce proteins involved in RNA metabolism, such as 2′,5′-oligoadenylate synthetase/RNase L, double stranded RNA-dependent protein kinase (PKR), melanoma differentiation associated gene-5 (mda-5), retinoic acid inducible gene-I (RIG-I) and hPNPase^{old-35} (97, 99, 114, 116). Inhibition of gene expression by IFNs at a post-transcriptional level has been described for the heavy chain of immunoglobulin mu (117), the IL-4 receptor (118) and c-myc (110, 111), the focus of the present studies.

The observation that type I IFN selectively reduces c-myc mRNA has been described in multiple studies using several model cell culture systems. Jonak and Knight first hypothesized that IFN-β mediated downregulation of c-myc mRNA might mediate growth inhibition in Daudi human lymphoblastoid cells (110). As a follow-up study, Dani et al. demonstrated in Daudi cells that IFN c/5 did not affect the transcription rate of c-myc mRNA, but rather reduced the half-life of this mRNA (111). A posttranscriptional destabilization of c-myc mRNA as a mechanism of type I IFN-mediated c-myc suppression has also been described in colon carcinoma cells (119). It was shown that during terminal differentiation of hematopoietic cells, autocrine IFN-β induces c-myc suppression and induces G₀/G₁ arrest in these cells (120). Additionally, previous studies from our laboratory documented that IFN-β and mezerein-induced terminal differentiation of human melanoma cells also correlated with downregulation of c-myc mRNA (121). Moreover, studies in different cell types consistently describe the ability of type I IFN to reduce c-myc expression. However, the effect of IFN-γ on c-myc expression varies in different cell contexts. In HeLa cells, treatment with IFN-α decreased while IFN-γ increased c-myc expression (122). In a murine myeloid cell line, IFN-γ inhibited c-myc gene expression by impairing the splicing process (123). Another report described the importance of Stat-1 in IFN-γ-mediated downregulation of c-myc. Studies employing wild type and Stat-1-null mouse embryonic fibroblasts identified a gamma activated sequence element in the c-myc promoter that was necessary, but not sufficient, to suppress c-myc expression in wild type cells (124).

Although a consensus exists that type I IFNs induce post-transcriptional modulation of c-myc mRNA, the molecular mechanism underlying this process is unclear. Different components of IFN-inducible RNA degradation machinery have been implicated in this action. In colon carcinoma cells, 2′,5′-oligoadenylate synthetase/RNase L system is believed to regulate IFN-β-mediated post-transcriptional processing of c-myc mRNA (119). In M1 murine myeloid leukemia cells, PKR has been shown to mediate type I IFN-induced c-myc suppression (125). A recent report indicates that in mouse monocyte/macrophage leukemia cells, IFN-β reduces steady state levels of Myc protein by increasing degradation through the 26S proteasome (126). In our previous studies, we revealed for the first time by employing recombinant hPNPase^{OLD-35} protein in in vitro mRNA degradation assays that a type I IFN-inducible exoribonuclease, hPNPase^{old-35}, could selectively degrade c-myc mRNA (102, 107). In the present studies, we now confirm that hPNPase^{OLD-35} is the enzyme responsible for IFN-β-mediated degradation of c-myc mRNA in human melanoma cells.

Myc is an important regulator of cell proliferation (127). Expression of exogenous Myc in cultured fibroblasts promotes S-phase entry and shortens the G₁ phase of the cell cycle, while activation of a conditional Myc is sufficient to drive quiescent cells into the cell cycle (128, 129). An association between c-myc downregulation and IFN-α-mediated G₀/G₁ arrest in Daudi cells was demonstrated (130) and it was shown that IFN-α-induced G₀/G₁ arrest correlated with upregulation of cyclin-dependent kinase inhibitors (CDKI), such as p21 and p15 early in this process and p27 in the late stage of growth arrest (131). Type I IFN treatment of Daudi cells induced p21 expression and G₁ arrest and these events were preceded by a strong reduction in c-myc levels (132). Myc can directly suppress the transcription of p21 and p27 and promote the ubiquitination of phosphorylated p27 (133-135). Our previous experiments confirm that overexpression of hPNPase^{old-35} downregulates c-myc and upregulates p27 (102) and in the present study we document that inhibition of hPNPase^{old-35} as well as overexpression of c-myc protects melanoma cells from IFN-β-mediated G₁ arrest. These findings firmly establish functional and mechanistic links between hPNPase^{old-35} induction by IFN-β, c-myc mRNA degradation by hPNPase^{old-35} and IFN-β-induced cell cycle arrest and eventual apoptosis (FIG. 26).

What is the practical significance of our observations? Myc is overexpressed in multiple tumor subtypes, including melanomas (127). The expression level of Myc inversely correlates with patient survival and thus may be used as a prognostic marker in cutaneous, subungual, acral lentigious, scalp, head and neck melanomas (136-141). Indeed, antisense inhibition of c-myc significantly inhibited the growth of melanoma cells in in vitro cultures (142) and improved the response to chemotherapy in human melanoma xenografts in nude mice (143). Type I IFNs have been used as adjuvant therapy for malignant melanoma with significant but limited success and high toxicity (144). Experimental overexpression of Myc in mouse fibroblasts and myeloblastic cells renders these cells resistant to cell cycle arrest by type I IFNs (145, 146). Uveal melanomas with high c-myc expression are also associated with IFN-α resistance (147). In these contexts and based on the poor survival of patients with malignant melanoma, improved therapies are mandated and cancer cell specific expression of hPNPase^{old-35}, by means of the telomerase or progression elevated gene-3 promoter (148, 149), might prove beneficial as an innovative adjuvant therapeutic approach that exploits the ability of hPNPase^{old-35} to degrade c-myc mRNA, thus inducing target cancer cell-specific growth arrest culminating in apoptosis.

### 9. REFERENCES

1. Leszczyniecka M, Kang D-C, Sarkar D, et al. Identification and cloning of human polynucleotide phosphorylase, hPNPase old-35, in the context of terminal differentiation and cellular senescence. Proc Natl Acad Sci USA 2002; 99: 16636-41.
2. Leszczyniecka M, DeSalle R, Kang D-C, Fisher PB. The origin of polynucleotide phosphorylase domains. Mol Phyl Evol 2004; 31: 123-30.
3. Leszczyniecka M, Su Z-Z, Kang D-C, Sarkar D, Fisher PB. Expression regulation and genomic organization of human polynucleotide phosphorylase, hPNPaseold-35, a type I interferon inducible early response gene. Gene 2003; 316: 143-56.
4. Sarkar D, Leszczyniecka M, Kang D-C, et al. Down-regulation of Myc as a potential target for growth arrest induced by human polynucleotide phosphorylase (hPNPaseold-35) in human melanoma cells. J Biol Chem 2003; 278: 24542-51.
5. Finkel T, Holbrook N J. Oxidants, oxidative stress and the biology of ageing. Nature 2000; 408: 239-47.
6. Harman D. Aging: a theory based on free radical and radiation chemistry. J Gerontol 1957; 2: 298-300.
7. Ames B N. Oxygen radicals and 8-hydroxyguanine in DNA. Jpn J Cancer Res 1991; 82: 1460-1.
8. Chen Q, Fischer A, Reagan J D, Yan L J, Ames B N. Oxidative DNA damage and senescence of human diploid fibroblast cells. Proc Natl Acad Sci USA 1995; 92: 4337-41.
9. Chen Q M. Replicative senescence and oxidant-induced premature senescence. Beyond the control of cell cycle checkpoints. Ann N Y Acad Sci 2000; 908: 111-25.
10. Toussaint O, Medrano E E, von Zglinicki T. Cellular and molecular mechanisms of stress-induced premature senescence (SIPS) of human diploid fibroblasts and melanocytes. Exp Gerontol 2000; 35: 927-45.
11. Irani K, Xia Y, Zweier J L, et al. Mitogenic signaling mediated by oxidants in Ras-transformed fibroblasts. Science 1997; 275: 1649-52.
12. Hagen T M, Yowe D L, Bartholomew J C, et al. Mitochondrial decay in hepatocytes from old rats: membrane potential declines, heterogeneity and oxidants increase. Proc Natl Acad Sci USA 1997; 94: 3064-9.
13. Schreck R, Albermann K, Baeuerle P A. Nuclear factor kappa B: an oxidative stress-responsive transcription factor of eukaryotic cells (a review). Free Radic Res Commun 1992; 17: 221-37.
14. Helenius M, Hanninen M, Lehtinen S K, Salminen A. Changes associated with aging and replicative senescence in the regulation of transcription factor nuclear factor-kappa B. Biochem J 1996; 318: 603-8.
15. Lavrovsky Y, Song C S, Chatterjee B, Roy A K. Age-dependent increase of heme oxygenase-1 gene expression in the liver mediated by NFkappaB. Mech Ageing Dev 2000; 114: 49-60.
16. Kim H J, Kim K W, Yu B P, Chung H Y. The effect of age on cyclooxygenase-2 gene expression: NF-kappaB activation and IkappaBalpha degradation. Free Radic Biol Med 2000; 28: 683-92.
17. Yan Z Q, Sirsjo A, Bochaton-Piallat M L, Gabbiani G, Hansson G K. Augmented expression of inducible NO synthase in vascular smooth muscle cells during aging is associated with enhanced NF-kappaB. activation Arterioscler Thromb Vasc Biol 1999; 19: 2854-62.
18. Xiao Z Q, Majumdar A P. Induction of transcriptional activity of AP-1 and NF-kappaB in the gastric mucosa during aging. Am J Physiol Gastrointest Liver Physiol 2000; 278: G855-65.
19. Korhonen P, Helenius M, Salminen A. Age-related changes in the regulation of transcription factor NF-kappa B in rat brain. Neurosci Lett 1997; 225: 61-4.
20. Helenius M, Hanninen M, Lehtinen S K, Salminen A. Aging-induced up-regulation of nuclear binding activities of oxidative stress responsive NF-kB transcription factor in mouse cardiac muscle. J Mol Cell Cardiol 1996; 28: 487-98.
21. Supakar P C, Jung M H, Song C S, Chatterjee B, Roy A K. Nuclear factor kappa B functions as a negative regulator for the rat androgen receptor gene and NF-kappa B activity increases during the age-dependent desensitization of the liver. J Biol Chem 1995; 270: 837-42.
22. Baldwin A S Jr. The NF-kappa B and I kappa B proteins: new discoveries and insights. Annu Rev Immunol 1996; 14: 649-83.
23. Finco T S, Baldwin A S. Mechanistic aspects of NF-kappa B regulation: the emerging role of phosphorylation and proteolysis. Immunity 1995; 3: 263-72.
24. Karin M, Ben-Neriah Y. Phosphorylation meets ubiquitination: the control of NF-[kappa]B activity. Annu Rev Immunol 2000; 18: 621-63.
25. Siebenlist U, Franzoso G, Brown K. Structure, regulation and function of NF-kappa B. Annu Rev Cell Biol 1994; 10: 405-55.
26. Chen F, Castranova V, Shi X, Demers L M. New insights into the role of nuclear factor-kappaB, a ubiquitous transcription factor in the initiation of diseases. Clin Chem 1999; 45: 7-17.
27. Piwowarski J, Grzechnik P, Dziembowski A, Dmochowska A, Minczuk M, Stepien P P. Human polynucleotide phosphorylase, hPNPase, is localized in mitochondria. J Mol Biol 2003; 329: 853-57.
28. Su Z-Z, Madireddi M T, Lin J J, et al. The cancer growth suppressor gene mda-7 selectively induces apoptosis in human breast cancer cells and inhibits tumor growth in nude mice. Proc Natl Acad Sci USA 1998; 95: 14400-5.
29. Valerie K. In: Wu-Pong S, Rojanasakul Y, editors. Biopharmaceutical Drug Design and Development. Humana Press, Totowa, N.J.; 1999. p. 69-142.
30. Baldwin A S Jr, Azizkhan J C, Jensen D E, Beg A A, Coodly L R. Induction of NF-kappa B DNA-binding activity during the G0-to-G1 transition in mouse fibroblasts. Mol Cell Biol 1991; 11: 4943-51.
31. Sarkar D, Kambe F, Hayashi Y, Ohmori S, Funahashi H, Seo H. Involvement of AP-1 and steroidogenic factor (SF)-1 in the cAMP-dependent induction of human adrenocorticotropic hormone receptor (ACTHR) promoter. Endocr J 2000; 47: 63-75.
32. Castedo M, Ferri K, Roumier T, Metivier D, Zamzami N, Kroemer G. Quantitation of mitochondrial alterations associated with apoptosis. J Immunol Methods 2002; 265: 39-47.
33. Kang D-C, Gopalkrishnan R V, Wu Q, Jankowsky E, Pyle A M, Fisher P B. mda-5: An interferon-inducible putative RNA helicase with double-stranded RNA-dependent ATPase activity and melanoma growth-suppressive properties. Proc Natl Acad Sci USA 2002; 99: 637-42.
34. Jiang H, Lin J J, Su Z-Z, Goldstein N I, Fisher P B. Subtraction hybridization identifies a novel melanoma differentiation associated gene, mda-7, modulated during human melanoma differentiation, growth and progression. Oncogene 1995; 11: 2477-86.
35. Su Z-Z, Shi Y, Fisher PB. Subtraction hybridization identifies a transformation progression-associated gene PEG-3 with sequence homology to a growth arrest and DNA damage-inducible gene. Proc Natl Acad Sci U S A 1997; 94: 9125-30.
36. Dimri G P, Campisi J. Altered profile of transcription factor-binding activities in senescent human fibroblasts. Exp Cell Res 1994; 212: 132-40.
37. Ponnappan U. Regulation of transcription factor NFkappa B in immune senescence. Front Biosci 1998; 3: D152-68.
38. Sun S C, Ganchi P A, Ballard D W, Greene W C. NF-kappa B controls expression of inhibitor I kappa B alpha: evidence for an inducible autoregulatory pathway. Science 1993; 259: 1912-5.
39. Hayakawa M, Miyashita H, Sakamoto 1, et al. Evidence that reactive oxygen species do not mediate NF-kappaB activation. EMBO J. 2003; 22: 3356-66.
40. Barnes P J, Karin M. Nuclear factor-kappaB: a pivotal transcription factor in chronic inflammatory diseases. N Engl J Med 1997; 336: 1066-71.
41. Schnabl B, Purbeck C A, Choi Y H, Hagedorn C H, Brenner D. Replicative senescence of activated human hepatic stellate cells is accompanied by a pronounced inflammatory but less fibrogenic phenotype. Hepatology 2003; 37: 653-64.
42. Csiszar A, Ungvari Z, Koller A, Edwards J G, Kaley G. Aging-induced proinflammatory shift in cytokine expression profile in coronary arteries. Faseb J 2003; 17: 1183-5.
43. Bruunsgaard H, Pedersen M, Pedersen B K. Aging and proinflaniuiatory cytokines. Curr Opin Hematol 2001; 8: 131-36.
44. Kiecolt-Glaser J K, Preacher K J, MacCallum R C, Atkinson C, Malarkey W B, Glaser R. Chronic stress and age-related increases in the proinflammatory cytokine IL-6. Proc Natl Acad Sci USA 2003; 100: 9090-5.
45. Ridker P M, Cushman M, Stampfer M J, Tracy R P, Hennekens C H. Inflammation, aspirin, and the risk of cardiovascular disease in apparently healthy men. N Engl J Med 1997; 336: 973-79.
46. Ito T, Ikeda U. Inflammatory cytokines and cardiovascular disease. Curr Drug Targets Inflamnm Allergy 2003; 2: 257-65.
47. Pradhan A D, Manson J E, Rifai N, Buring J E, Ridker P M. C-reactive protein, interleukin 6, and risk of developing type 2 diabetes mellitus. JAMA 2001; 286: 327-34.
48. Ferrucci L, Harris T B, Guralnik J M, et al. Serum IL-6 level and the development of disability in older persons. J Am Geriatr Soc 1999; 47: 639-46.
49. Hageman G J, Larik I, Pennings H J, Haenen G R, Wouters E F, Bast A. Systemic poly(ADP-ribose) polymerase-1 activation, chronic inflammation, and oxidative stress in COPD patients. Free Radic Biol Med 2003; 35: 140-48.
50. Mukaida N. Pathophysiological roles of interleukin-8/CXCL8 in pulmonary diseases. Am J Physiol Lung Cell Mol Physiol 2003; 284: L566-77.
51. Juarranz M G, Santiago B, Torroba M, et al. Vasoactive intestinal peptide modulates proinflammatory mediator synthesis in osteoanthritic and rheumatoid synovial cells. Rheumatol 2004; 43: 416-22.
52. Alcorta, D. A., Y. Xiong, D. Phelps, G. Hannon, D. Beach, and J. C. Barrett. 1996. Involvement of the cyclin-dependent kinase inhibitor p 16 (INK4a) in replicative senescence of normal human fibroblasts. Proc Natl Acad Sci USA 93:13742-7.
53. Baginsky, S., A. Shteiman-Kotler, V. Liveanu, S. Yehudai-Resheff, M. Bellaoui, R. E. Settlage, J. Shabanowitz, D. F. Hunt, G. Schuster, and W. Gruissem. 2001. Chloroplast PNPase exists as a homo-multimer enzyme complex that is distinct from the Escherichia coli degradosome. Rna 7:1464-75.
54. Campisi, J. 1992. Gene expression in quiescent and senescent fibroblasts. Ann N Y Acad Sci 663:195-201.
55. Carpousis, A. J., G. Van Houwe, C. Ehretsmann, and H. M. Krisch. 1994. Copurification of E. coli RNAase E and PNPase: evidence for a specific association between two enzymes important in RNA processing and degradation. Cell 76:889-900.
56. Chin, L. 2003. The genetics of malignant melanoma: lessons from mouse and man. Nat Rev Cancer 3:559-70.
57. Collado, M., R. H. Medema, I. Garcia-Cao, M. L. Dubuisson, M. Barradas, J. Glassford, C. Rivas, B. M. Burgering, M. Serrano, and E. W. Lam. 2000. Inhibition of the phosphoinositide 3-kinase pathway induces a senescence-like arrest mediated by p27Kip1. J Biol Chem 275:21960-8.
58. Deutscher, M. P. 1993. Promiscuous exoribonucleases of Escherichia coli. J Bacteriol 175:4577-83.
59. Deutscher, M. P. 1993. Ribonuclease multiplicity, diversity, and complexity. J Biol Chem 268:13011-4.
60. Deutscher, M. P., and Z. Li. 2001. Exoribonucleases and their multiple roles in RNA metabolism. Prog Nucleic Acid Res Mol Biol 66:67-105.
61. Dimri, G. P., X. Lee, G. Basile, M. Acosta, G. Scott, C. Roskelley, E. E. Medrano, M. Linskens, I. Rubelj, O. Pereira-Smith, and et al. 1995. A biomarker that identifies senescent human cells in culture and in aging skin in vivo. Proc Natl Acad Sci USA 92:9363-7.
62. Fisher, P. B., and S. Grant. 1985. Effects of interferon on differentiation of normal and tumor cells. Pharmacol Ther 27:143-66.
63. Fisher, P. B., D. R. Prignoli, H. Hermo, Jr., I. B. Weinstein, and S. Pestka. 1985. Effects of combined treatment with interferon and mezerein on melanogenesis and growth in human melanoma cells. J Interferon Res 5:11-22.
64. Graham, G. M., L. Guarini, T. A. Moulton, S. Datta, S. Ferrone, P. Giacomini, R. S. Kerbel, and P. B. Fisher. 1991. Potentiation of growth suppression and modulation of the antigenic phenotype in human melanoma cells by the combination of recombinant human fibroblast and immune interferons. Cancer Immunol Immunother 32:382-90.
65. Grandori, C., S. M. Cowley, L. P. James, and R. N. Eisenman. 2000. The Myc/Max/Mad network and the transcriptional control of cell behavior. Annu Rev Cell Dev Biol 16:653-99.
66. Grunberg-Manago, M. 1999. Messenger RNA stability and its role in control of gene expression in bacteria and phages. Annu Rev Genet. 33:193-227.
67. Guarini, L., G. M. Graham, H. Jiang, S. Ferrone, S. Zucker, and P. B. Fisher. 1992. Modulation of the antigenic phenotype of human melanoma cells by differentiation-inducing and growth-suppressing agents. Pigment Cell Res Suppl 2:123-31.
68. Hayflick, L. 1976. The cell biology of human aging. N Engl J Med 295:1302-8.
69. Holmes, M., E. Rosenberg, and K. Valerie. 2003. Adenovirus expressing p53, p1-16. In S. Deb and S. P. Deb (ed.), Methods in Molecular Biology, vol. 234. Humana Press, Totowa, N.J.
70. Jarrige, A., D. Brechemier-Baey, N. Mathy, O. Duche, and C. Portier. 2002. Mutational analysis of polynucleotide phosphorylase from Escherichia coli. J Mol Biol 321:397-409.
71. Jarrige, A. C., N. Mathy, and C. Portier. 2001. PNPase autocontrols its expression by degrading a double-stranded structure in the pnp mRNA leader. EMBO J. 20:6845-55.
72. Jiang, H., Z. Z. Su, J. Boyd, and P. B. Fisher. 1993. Gene expression changes associated with reversible growth suppression and the induction of terminal differentiation in human melanoma cells. Mol Cell Differ 1:41-66.
73. Kelly, K. O., N. B. Reuven, Z. Li, and M. P. Deutscher. 1992. RNase PH is essential for tRNA processing and viability in RNase-deficient Escherichia coli cells. J Biol Chem 267:16015-8.
74. Leszczyniecka, M., R. DeSalle, D.C. Kang, and P. B. Fisher. 2004. The origin of polynucleotide phosphorylase domains. Mol Phyl Evol 31: 123-130.
75. Leszczyniecka, M., D. C. Kang, D. Sarkar, Z. Z. Su, M. Holmes, K. Valerie, and P. B. Fisher. 2002. Identification and cloning of human polynucleotide phosphorylase, hPNPaseold-35, in the context of terminal differentiation and cellular senescence. Proc Natl Acad Sci USA 99:16636-41.
76. Leszczyniecka, M., Z. Z. Su, D.C. Kang, D. Sarkar, and P. B. Fisher. 2003. Expression regulation and genomic organization of human polynucleotide phosphorylase, hPNPaseold-35, a type I interferon inducible early response gene. Gene 316: 143-156.
77. O'Hagan, R. C., M. Ohh, G. David, I. M. de Alboran, F. W. Alt, W. G. Kaelin, Jr., and R. A. DePinho. 2000. Myc-enhanced expression of Cull promotes ubiquitin-dependent proteolysis and cell cycle progression. Genes Dev 14:2185-91.
78. Obaya, A. J., M. K. Mateyak, and J. M. Sedivy. 1999. Mysterious liaisons: the relationship between c-Myc and the cell cycle. Oncogene 18:2934-41.
79. Piwowarski, J., P. Grzechnik, A. Dziembowski, A. Dmochowska, M. Minczuk, and P. P. Stepien. 2003. Human polynucleotide phosphorylase, hPNPase, is localized in mitochondria. J Mol Biol 329:853-7.
80. Raijmakers, R., W. V. Egberts, W. J. van Venrooij, and G. J. Pruijn. 2002. Protein-protein interactions between human exosome components support the assembly of RNase PH-type subunits into a six-membered PNPase-like ring. J Mol Biol 323:653-63.
81. Reuven, N. B., and M. P. Deutscher. 1993. Substitution of the 3′ terminal adenosine residue of transfer RNA in vivo. Proc Natl Acad Sci USA 90:4350-3.
82. Sarkar, D., I. V. Lebedeva, L. Emdad, D.C. Kang, A. S. Baldwin, Jr., and P. B. Fisher. 2004. Human polynucleotide phosphorylase (hPNPaseold-35): a potential link between aging and inflammation. Cancer Res 64:7473-8.
83. Sarkar, D., M. Leszczyniecka, D.C. Kang, I. V. Lebedeva, K. Valerie, S. Dhar, T. K. Pandita, and P. B. Fisher. 2003. Down-regulation of Myc as a potential target for growth arrest induced by human polynucleotide phosphorylase (hPNPaseold-35) in human melanoma cells. J Biol Chem 278:24542-51.
84. Sarkar, D., Z. Z. Su, I. V. Lebedeva, M. Sauane, R. V. Gopalkrishnan, K. Valerie, P. Dent, and P. B. Fisher. 2002. mda-7 (IL-24) Mediates selective apoptosis in human melanoma cells by inducing the coordinated overexpression of the GADD family of genes by means of p38 MAPK. Proc Natl Acad Sci USA 99:10054-9.
85. Serrano, M., and M. A. Blasco. 2001. Putting the stress on senescence. Curr Opin Cell Biol 13:748-53.
86. Sherwood, S. W., D. Rush, J. L. Ellsworth, and R. T. Schimke. 1988. Defining cellular senescence in IMR-90 cells: a flow cytometric analysis. Proc Natl Acad Sci USA 85:9086-90.
87. Stein, G. H., L. F. Drullinger, A. Soulard, and V. Dulic. 1999. Differential roles for cyclin-dependent kinase inhibitors p21 and p 16 in the mechanisms of senescence and differentiation in human fibroblasts. Mol Cell Biol 19:2109-17.
88. Symmons, M. F., G. H. Jones, and B. F. Luisi. 2000. A duplicated fold is the structural basis for polynucleotide phosphorylase catalytic activity, processivity, and regulation. Structure Fold Des 8:1215-26.
89. Symmons, M. F., M. G. Williams, B. F. Luisi, G. H. Jones, and A. J. Carpousis. 2002. Running rings around RNA: a superfamily of phosphate-dependent RNases. Trends Biochem Sci 27:11-8.
90. Yang, W., J. Shen, M. Wu, M. Arsura, M. FitzGerald, Z. Suldan, D. W. Kim, C. S. Hofmann, S. Pianetti, R. Romieu-Mourez, L. P. Freedman, and G. E. Sonenshein. 2001. Repression of transcription of the p27(Kip1) cyclin-dependent kinase inhibitor gene by c-Myc. Oncogene 20:1688-702.
91. Yehudai-Resheff, S., V. Portnoy, S. Yogev, N. Adir, and G. Schuster. 2003. Domain analysis of the chloroplast polynucleotide phosphorylase reveals discrete functions in RNA degradation, polyadenylation, and sequence homology with exosome proteins. Plant Cell 15:2003-19.
92. Yoon, G., H. J. Kim, Y. S. Yoon, H. Cho, I. K. Lim, and J. H. Lee. 2002. Iron chelation-induced senescence-like growth arrest in hepatocyte cell lines: association of transforming growth factor beta1 (TGF-beta1)-mediated p27Kip1 expression. Biochem J 366:613-21.
93. Fisher PB and Grant S (1985) Effects of interferon on differentiation of normal and tumor cells. Pharmacol. Ther. 27: 143-166
94. Fisher P B, Hermo H, Jr., Solowey W E, Dietrich M C, Edwalds G M, Weinstein I B, Langer J A, Pestka S, Giacomini P, Kusama M, Ferrone S (1986) Effect of recombinant human fibroblast interferon and mezerein on growth, differentiation, immune interferon binding and tumor associated antigen expression in human melanoma cells. Anticancer Res. 6: 765-774
95. Barber G N (2001) Host defense, viruses and apoptosis. Cell Death Differ. 8: 113-126
96. Leszczyniecka M, Roberts T, Dent P, Grant S and Fisher PB (2001) Differentiation therapy of human cancer: basic science and clinical applications. Pharmacol. Ther. 90: 105-156
97. Stark G R, Kerr I M, Williams B R, Silverman R H and Schreiber R D (1998) How cells respond to interferons. Annu. Rev. Biochem. 67: 227-264
98. Pestka S (1997) The interferon receptors. Semin. Oncol. 24: S9-18-S9-40
99. Leszczyniecka M, Kang D C, Sarkar D, Su Z Z, Holmes M, Valerie K and Fisher P B (2002) Identification and cloning of human polynucleotide phosphorylase, hPNPaseold-35, in the context of terminal differentiation and cellular senescence. Proc. Natl. Acad. Sci. U.S.A. 99: 16636-16641
100. Leszczyniecka M, Su Z Z, Kang D C, Sarkar D and Fisher P B (2003) Expression regulation and genomic organization of human polynucleotide phosphorylase, hPNPaseold-35, a type I interferon inducible early response gene. Gene 316: 143-156
101. Leszczyniecka M, DeSalle R, Kang D C and Fisher P B (2003) The origin of polynucleotide phosphorylase domains. Mol. Phyl. Evol. 31: 123-130
102. Sarkar D, Leszczyniecka M, Kang D C, Lebedeva I V, Valerie K, Dhar S, Pandita T K and Fisher P B (2003) Down-regulation of Myc as a potential target for growth arrest induced by human polynucleotide phosphorylase (hPNPaseold-35) in human melanoma cells. J. Biol. Chem. 278: 24542-24551
103. Fisher P B, Prignoli D R, Hermo H, Jr., Weinstein I B and Pestka S (1985) Effects of combined treatment with interferon and mezerein on melanogenesis and growth in human melanoma cells. J. Interferon Res. 5: 11-22
104. Jiang H, Lin J and Fisher P B (1994) A molecular definition of terminal cell differentiation in human melanoma cells. Mol. Cell. Differ. 2: 221-239
105. Campisi J (1996) Replicative senescence: an old lives' tale? Cell 84: 497-4500
106. Serrano M and Blasco M A (2001) Putting the stress on senescence. Curr. Opin. Cell Biol. 13: 748-753
107. Sarkar D, Park E S, Emdad L, Randolph A, Valerie K and Fisher P B (2005) Defining the domains of human polynucleotide phosphorylase (hPNPaseold-35) mediating cellular senescence. Mol. Cell. Biol. 25: 7333-7343
108. Sarkar D, Lebedeva I V, Emdad L, Kang D C, Baldwin A S, Jr. and Fisher P B (2004) Human polynucleotide phosphorylase (hPNPaseold-35): a potential link between aging and inflammation. Cancer Res. 64: 7473-7478
109. Piwowarski J, Grzechnik P, Dziembowski A, Dmochowska A, Minczuk M and Stepien P P (2003) Human polynucleotide phosphorylase, hPNPase, is localized in mitochondria. J. Mol. Biol. 329: 853-857
110. Jonak G J and Knight E, Jr (1984) Selective reduction of c-myc mRNA in Daudi cells by human beta interferon. Proc. Natl. Acad. Sci. U.S.A. 81: 1747-1750
111. Dani C, Mechti N, Piechaczyk M, Lebleu B, Jeanteur P and Blanchard J M (1985) Increased rate of degradation of c-myc mRNA in interferon-treated Daudi cells. Proc. Natl. Acad. Sci. U.S.A. 82: 4896-4899
112. Pellegrini S, John J, Shearer M, Kerr I M and Stark G R (1989) Use of a selectable marker regulated by alpha interferon to obtain mutations in the signaling pathway. Mol. Cell. Biol. 9: 4605-4612
113. Darnell J E, Jr., Kerr I M and Stark G R (1994) Jak-STAT pathways and transcriptional activation in response to IFNs and other extracellular signaling proteins. Science 264: 1415-1421
114. Kang D C, Gopalkrishnan R V, Wu Q, Jankowsky E, Pyle A M and Fisher P B (2002) mda-5: An interferon-inducible putative RNA helicase with double-stranded RNA-dependent ATPase activity and melanoma growth-suppressive properties. Proc. Natl. Acad. Sci. U.S.A. 99: 637-642
115. de Veer M J, Holko M, Frevel M, Walker E, Der S, Paranjape J M, Silverman R H and Williams B R (2001) Functional classification of interferon-stimulated genes identified using microarrays. J. Leukoc. Biol. 69: 912-920
116. Yoneyama M, Kikuchi M, Natsukawa T, Shinobu N, Imaizumi T, Miyagishi M, Taira K, Akira S and Fujita T (2004) The RNA helicase RIG-I has an essential function in double-stranded RNA-induced innate antiviral responses. Nat. Immunol. 5: 730-737
117. Meurs E and Hovanessian A G (1988) Alpha-interferon inhibits the expression of heavy chain mu messenger RNA in Daudi cells. Embo J. 7: 1689-1696
118. So E Y, Park H H and Lee C E (2000) IFN-gamma and IFN-alpha posttranscriptionally down-regulate the IL-4-induced IL-4 receptor gene expression. J. Immunol. 165: 5472-5479
119. Chatterjee D and Savarese T M (1992) Posttranscriptional regulation of c-myc proto-oncogene expression and growth inhibition by recombinant human interferon-beta ser17 in a human colon carcinoma cell line. Cancer Chemother. Pharmacol. 30: 12-20
120. Resnitzky D, Yarden A, Zipori D and Kimchi A (1986) Autocrine beta-related interferon controls c-myc suppression and growth arrest during hematopoietic cell differentiation. Cell 46: 31-40
121. Jiang H, Lin J, Young S M, Goldstein N I, Waxman S, Davila V, Chellappan SP and Fisher PB (1995) Cell cycle gene expression and E2F transcription factor complexes in human melanoma cells induced to terminally differentiate. Oncogene 11: 1179-1189
122. Kelly J M, Gilbert C S, Stark G R and Kerr I M (1985) Differential regulation of interferon-induced mRNAs and c-myc mRNA by alpha- and gamma-interferons. Eur. J. Biochem. 153: 367-371
123. Harel-Bellan A, Brini A T and Farrar W L (1988) IFN-gamma inhibits c-myc gene expression by impairing the splicing process in a colony-stimulating factor dependent murine myeloid cell line. J Immunol 141: 1012-1017
124. Ramana C V, Grammatikakis N, Chernov M, Nguyen H, Goh K C, Williams B R and Stark G R (2000) Regulation of c-myc expression by IFN-gamma through Stat1-dependent and -independent pathways. Embo J. 19: 263-272
125. Raveh T, Hovanessian A G, Meurs E F, Sonenberg N and Kimchi A (1996) Double-stranded RNA-dependent protein kinase mediates c-Myc suppression induced by type I interferons. J. Biol. Chem. 271: 25479-25484
126. Hu X, Bies J and Wolff L (2005) Interferon beta increases c-Myc proteolysis in mouse monocyte/macrophage leukemia cells. Leuk. Res. in press
127. Grandori C, Cowley S M, James L P and Eisenman R N (2000) The Myc/Max/Mad network and the transcriptional control of cell behavior. Annu. Rev. Cell Dev. Biol. 16: 653-699
128. Karn J, Watson J V, Lowe A D, Green S M and Vedeckis W (1989) Regulation of cell cycle duration by c-myc levels. Oncogene 4: 773-787
129. Eilers M, Picard D, Yamamoto K R and Bishop J M (1989) Chimaeras of myc oncoprotein and steroid receptors cause hormone-dependent transformation of cells. Nature 340: 66-68
130. Einat M, Resnitzky D and Kimchi A (1985) Close link between reduction of c-myc expression by interferon and, G0/G1 arrest. Nature 313: 597-600
131. Sangfelt O, Erickson S, Castro J, Heiden T, Gustafsson A, Einhom S and Grander D (1999) Molecular mechanisms underlying interferon-alpha-induced G0/G1 arrest: CKI-mediated regulation of G1 Cdk-complexes and activation of pocket proteins. Oncogene 18: 2798-2810
132. Subramaniam P S, Cruz P E, Hobeika A C and Johnson H M (1998) Type I interferon induction of the Cdk-inhibitor p21 WAF1 is accompanied by ordered G1 arrest, differentiation and apoptosis of the Daudi B-cell line. Oncogene 16: 1885-1890
133. Yang W, Shen J, Wu M, Arsura M, FitzGerald M, Suldan Z, Kim D W, Hofmann C S, Pianetti S, Romieu-Mourez R, Freedman L P and Sonenshein G E (2001) Repression of transcription of the p27(Kip1) cyclin-dependent kinase inhibitor gene by c-Myc. Oncogene 20: 1688-1702
134. Claassen G F and Hann S R (2000) A role for transcriptional repression of p21CIP1 by c-Myc in overcoming transforming growth factor beta-induced cell-cycle arrest. Proc. Natl. Acad. Sci. U.S.A. 97: 9498-9503
135. Obaya A J, Mateyak M K and Sedivy J M (1999) Mysterious liaisons: the relationship between c-Myc and the cell cycle. Oncogene 18: 2934-2941
136. Ross D A and Wilson G D (1998) Expression of c-myc oncoprotein represents a new prognostic marker in cutaneous melanoma. Br. J. Surg. 85: 46-51
137. Grover R, Grobbelaar A O, Hudson D A, Forder M, Wilson G D and Sanders R (1997) The clinical significance of oncogene expression in subungual melanoma. Br. J. Plast. Surg. 50: 15-19
138. Grover R, Ross D A, Wilson G D and Sanders R (1997) Measurement of c-myc oncoprotein provides an independent prognostic marker for regional metastatic melanoma. Br. J. Plast. Surg. 50: 478-482
139. Grover R, Chana J, Grobbelaar A O, Hudson D A, Forder M, Wilson G D and Sanders R (1999) Measurement of c-myc oncogene expression provides an accurate prognostic marker for acral lentiginous melanoma. Br. J. Plast. Surg. 52: 122-126
140. Chana J S, Grover R, Wilson G D, Hudson D A, Forders M, Sanders R and Grobbelaar A O (1998) The clinical significance of c-myc oncogene expression in melanomas of the scalp. Br. J. Plast. Surg. 51: 191-194
141. Chana J S, Grover R, Wilson G D, Hudson D A, Forders M, Sanders R and Grobbelaar A O (2001) The prognostic importance of c-myc oncogene expression in head and neck melanoma. Ann. Plast. Surg. 47: 172-177
142. Chana J S, Grover R, Tulley P, Lohrer H, Sanders R, Grobbelaar A O and Wilson G D (2002) The c-myc oncogene: use of a biological prognostic marker as a potential target for gene therapy in melanoma. Br. J. Plast. Surg. 55: 623-627
143. Zupi G, Scarsella M, Semple S C, Mottolese M, Natali P G and Leonetti C (2005) Antitumor efficacy of bcl-2 and c-myc anti sense oligonucleotides in combination with cisplatin in human melanoma xenografts: relevance of the administration sequence. Clin. Cancer Res. 11: 1990-1998
144. McClay E F (2002) Adjuvant therapy for patients with high-risk malignant melanoma. Semin. Oncol. 29: 389-399
145. Einat M and Kimchi A (1988) Transfection of fibroblasts with activated c-myc confers resistance to antigrowth effects of interferon. Oncogene 2: 485-491
146. Resnitzky D and Kimchi A (1991) Deregulated c-myc expression abrogates the interferon- and interleukin 6-mediated G0/G1 cell cycle arrest but not other inhibitory responses in M1 myeloblastic cells. Cell. Growth Differ. 2: 33-41
147. Tulley P N, Neale M, Jackson D, Chana J S, Grover R, Cree I, Grobbelaar A O and Wilson G D (2004) The relation between c-myc expression and interferon sensitivity in uveal melanoma. Br. J. Ophthalmol. 88: 1563-1567
148. Gu J and Fang B (2003) Telomerase promoter-driven cancer gene therapy. Cancer Biol. Ther. 2: S64-70
149. Su Z Z, Sarkar D, Emdad L, Duigou G J, Young C S, Ware J, Randolph A, Valerie K and Fisher P B (2005) Targeting gene expression selectively in cancer cells by using the progression-elevated gene-3 promoter. Proc. Natl. Acad. Sci. U.S.A. 102: 1059-1064
150. Lebedeva I V, Su Z Z, Chang Y, Kitada S, Reed J C and Fisher P B (2002) The cancer growth suppressing gene mda-7 induces apoptosis selectively in human melanoma cells. Oncogene 21: 708-718

Various publications are cited herein, the contents of which are hereby incorporated by reference in their entireties.

1-44. (canceled)

45. A method for identifying an agent that inhibits inflammation, comprising administering a test agent that is a putative anti-inflammatory agent to a system comprising an old-35 promoter element operatively linked to a reporter gene and determining whether the exposure to the test agent increases transcription of the reporter gene, wherein a decrease in transcription of the reporter gene indicates that the test agent inhibits inflammation.

46. The method of claim 45, wherein the old-35 promoter comprises a sequence as set forth in SEQ ID NO: 2.

47. The method of claim 45, wherein the old-35 promoter comprises a sequence as set forth in SEQ ID NO:4.

48. The method of claim 45, wherein the reporter gene is selected from the group consisting of green fluorescent protein and luciferase.

49. A method for identifying an agent that inhibits inflammation, comprising administering a test agent to a cell comprising an old-35 gene operatively linked to a promoter element, wherein the old-35 gene is transcribed and expressed as OLD-35 protein, and determining whether the exposure to the test agent decreases the amount of reactive oxygen species in the cell.

50. The method of claim 49, wherein the OLD-35 protein has a sequence as set forth in SEQ ID NO: 6.

51. The method of claim 49, wherein the cell is a test cell into which an old-35 gene operatively linked to a promoter element has been introduced, and the amount of reactive oxygen species in the test cell exposed to the test agent is decreased relative to the amount of reactive oxygen species in a first control cell into which the old-35 gene operatively linked to the promoter has been introduced, but where the first control cell is not exposed to the test agent; wherein the amount of reactive oxygen species in the first control cell is greater than that in a second control cell which does not contain the old-35 gene operatively linked to the promoter element and which is not senescent or terminally differentiated.

52. The method of claim 51, wherein the OLD-35 protein has a sequence as set forth in SEQ ID NO: 6.

53. A kit for detecting inflammation in a subject, comprising a probe that binds to an old-35 gene product selected from the group consisting of old-35 mRNA and OLD-35 protein and a probe that binds to a cytokine selected from the group consisting of interleukin-6, interleukin-8, TNFR1, RANTES and MMP-3.

54. A method of inhibiting inflammation in a subject in need of such treatment, comprising administering, to the subject, an effective amount of an agent selected from the group consisting of an antibody that binds to OLD-35 protein, an old-35 RNA-i, an antisense old-35 nucleic acid, and a nucleic acid comprising an old-35 promoter element operatively linked to a gene that inhibits inflammation.

55. An anti-inflammatory composition, comprising an agent that antagonizes old-35 activity selected from the group consisting of an old-35 RNA-i, an old-35 antisense RNA, and an antibody directed toward OLD-35, and another anti-inflammatory agent.

56. An OLD-35 variant protein comprising one, but not two, RPH domain and having an activity selected from the group consisting of anti-proliferative activity, PNPase activity, RNA degradation activity, cell-cycle slowing activity, senescence-inducing activity, immunity-inducing activity, and a combination thereof.

57. The OLD-35 variant of claim 56 comprising amino acid residues 52-183 (SEQUENCE ID NO: 15) of native OLD-35 protein, or a sequence that is at least 90 percent, preferably at least 95 percent homologous to residues 52-183.

58. The OLD-35 variant of claim 56 comprising amino acid residues 366-501 (SEQUENCE ID NO: 16) of native OLD-35 protein, or a sequence that is at least 90 percent, preferably at least 95 percent homologous to residues 366-501.

59. The OLD-35 variant of claim 57, further comprising residues 289-363 (SEQUENCE ID NO:21) of native OLD-35 protein, or a sequence that is at least 90 percent, preferably at least 95 percent homologous to residues 289-363.

60. The OLD-35 variant of claim 58, further comprising residues 289-363 (SEQUENCE ID NO:21) of native OLD-35 protein, or a sequence that is at least 90 percent, preferably at least 95 percent homologous to residues 289-363.

61. A method for identifying an agent that inhibits inflammation, comprising administering a test agent to a cell comprising an old-35 gene operatively linked to a promoter element, wherein the old-35 gene is transcribed and expressed as OLDS 5 protein, and determining whether the exposure to the test agent decreases the amount of binding between NF-[kappa]B and its target sequence in the cell.

62. The method of claim 61, wherein the OLD-35 protein has a sequence as set forth in SEQ ID NO: 6.

63. The method of claim 61, wherein the cell is a test cell into which an old-35 gene operatively linked to a promoter element has been introduced, and the amount of binding between NF-KB and its target sequence in the test cell exposed to the test agent is decreased relative to the amount of binding between NF-[kappa]B and its target sequence in a first control cell into which the old-35 gene operatively linked to the promoter has been introduced, but where the first control cell is not exposed to the test agent; wherein the amount of binding between NF-[kappa]B and its target sequence in the first control cell is greater than that in a second control cell which does not contain the old-35 gene operatively linked to the promoter element and which is not senescent or terminally differentiated.

64. The method of claim 63, wherein the OLD-35 protein has a sequence as set forth in SEQ ID NO: 6.

65. A method for identifying an agent that inhibits inflammation, comprising administering a test agent to a cell comprising an old-35 gene operatively linked to a promoter element, wherein the old-35 gene is transcribed and expressed as OLD-35 protein, and determining whether the exposure to the test agent decreases the amount of translocation of aNF-[kappa]B protein from the cytoplasm into the nucleus of the cell.

66. The method of claim 65, wherein the OLD-35 protein has a sequence as set forth in SEQ ID NO: 6.

67. The method of claim 65 wherein the cell is a test cell into which an old-35 gene operatively linked to a promoter element has been introduced, and the amount of translocation of a NF-[kappa]B protein from the cytoplasm into the nucleus in the test cell exposed to the test agent is decreased relative to the amount of translocation of a NF-[kappa]B protein from the cytoplasm into the nucleus in a first control cell into which the old-35 gene operatively linked to the promoter has been introduced, but where the first control cell is not exposed to the test agent; wherein the amount of translocation of a NF-[kappa]B protein from the cytoplasm into the nucleus in the first control cell is greater than that in a second control cell which does not contain the old-35 gene operatively linked to the promoter element and which is not senescent or terminally differentiated.

68. The method of claim 67, wherein the OLD-35 protein has a sequence as set forth in SEQ ID NO: 6.

69. A method for identifying an agent that inhibits inflammation, comprising administering a test agent to a cell comprising an old-35 gene operatively linked to a promoter element, wherein the old-35 gene is transcribed and expressed as OLD-35 protein, and determining whether the exposure to the test agent decreases the amount of a cytokine selected from the group consisting of interleukin-6, interleukin-8, TNFR1, RANTES and MMP-3 in the cell.

70. The method of claim 69, wherein the OLD-35 protein has a sequence as set forth in SEQ ID NO: 6.

71. The method of claim 69 wherein the cell is a test cell into which an old-35 gene operatively linked to a promoter element has been introduced, and the amount of a cytokine selected from the group consisting of interleukin-6, interleukin-8, TNFR1, RANTES and MMP-3 in the test cell exposed to the test agent is decreased relative to the amount of a cytokine selected from the group consisting of interleukin-6, interleukin-8, TNFR1, RANTES and MMP-3 in a first control cell into which the old-35 gene operatively linked to the promoter has been introduced, but where the first control cell is not exposed to the test agent; wherein the amount of a cytokine selected from the group consisting of interleukin-6, interleukin-8, TNFR1, RANTES and MMP-3 in the first control cell is greater than that in a second control cell which does not contain the old-35 gene operatively linked to the promoter element and which is not senescent or terminally differentiated.

72. The method of claim 71, wherein the OLD-35 protein has a sequence as set forth in SEQ ID NO: 6.

73. A model system of arthritis, comprising a non-human animal carrying a transgene comprising an old-35 gene operatively linked to a promoter element that is selectively active in cells comprised in a joint of the animal.

74. A model system for atherosclerosis, comprising a non-human animal carrying a transgene comprising an old-35 gene operatively linked to a promoter element that is selectively active in cells of the vascular system.

75. A model system for Alzheimer's disease, comprising a non-human animal carrying a transgene comprising an old-35 gene operatively linked to a promoter element that is selectively active in cells of the central nervous system.

76. A method for evaluating inflammation in a transgenic non-human animal carrying a transgene comprising an old-35 gene operatively linked to a promoter element, comprising determining, in a cell, tissue, or fluid of the animal, whether the amount of reactive oxygen species is increased.

77. A method for evaluating inflammation in a transgenic non-human animal carrying a transgene comprising an old-35 gene operatively linked to a promoter element, comprising determining, in a cell of the animal, whether the amount of binding of a NF-[kappa]B protein to its target sequence is increased.

78. A method for evaluating inflammation in a transgenic non-human animal carrying a transgene comprising an old-35 gene operatively linked to a promoter element, comprising determining, in a cell of the animal, whether the amount of a NF-[kappa]B protein translocated into the nucleus is increased.

79. A method for evaluating inflammation in a transgenic non-human animal carrying a transgene comprising an old-35 gene operatively linked to a promoter element, comprising determining, in a cell, tissue, or fluid of the animal, whether the amount of a cytokine selected from the group consisting of interleukin-6, interleukin-8, TNFR1, RANTES and MMP-3 is increased.

80. A method of detecting inflammation in a subject, comprising determining whether there is an increase in the expression of an old-35 gene in a cell of the subject relative to a control cell.

81. A kit for detecting inflammation in a subject, comprising a probe that binds to an old-35 gene product selected from the group consisting of old-55 mRNA and OLD-35 protein and a probe that binds to a cytokine mRNA selected from the group consisting of interleukin-6 mRNA, interleukin-8 mRNA, TNFR1 mRNA, RANTES mRNA and MMP-3 mRNA.


SEQUENCE LISTING (41 sequences)

SEQ ID NO: 1 - DNA, 4052 bp, Homo Sapien
cttatagtctatacaaagcagtttaaattttatccttaaagatttaggaagccactgaaaaaatttaatcaggatggagacacaattctatttgtgattgagaaagaagatccttctggtagtggtgtagcacagactttcttaaagttgagtaggctacattctttcaagagaagaattttttcctgaacacttcagggactgctgatacttttaattataatgttactatataacaaaatataacttccatattgttacagcttttagtcataaaagcaatataaatgtatatcctgtctaaactaaactacaaactaaattaaaatatagttaaagggccgggctctgtggctcacgcctgtaatcccagcattttgtgaggccaaggcaggcagatcacctgagggtgggagttggagatcagcctggccaacatggtgaaaccctgtctctattaaaagtacaaaaattagctgggcgcggtggcgcacgtctgtagtcccagctacttgggaggctgaggtaggagaattgcttgaacccaggaggtggaggttgcagtgagccaagattgtgccactgcactccagcttgggcaacagagcaagactccatctcaaaaaaaaaaaaagtagttaagcataatttaatgcaatggtgttttacttaacgtagttgcttattcagaacatgcagtggtattaactgttttgatgcaggttcatcagagtttgaagaaaagggaaaaggagatctgagaagttgctggtggatttaagttggatcatttttaatgtccttcagcagtcataggtaaataaatcccttttaaattatattttggctgttgaactacacttgacataactctacttgcagaataacatctcagactgaacctcacggattatagcttcagtagtaacaactacaagaatttaaacttattatatttttctttctacgctaatctaaaaatgcttaatataaacatataaacgcattctgtatcatctatatcttcataataaagctctgctgcaagattttagtgcctacatttgcttttgagtttactatccaattactactatttgaattatatcagatttgtgtgaggaaatgggggtgggggattcttaataggtaaatgctgctttccatcacttctggctggataaaagctaaccgattcaaggagttcaaggagttctgaagaagagaaaaatgttcagaatacctggaaactggaggaagaattaagaaaagtctggataagcctgggcgcggtggctcatgcctgtaatcccagcactttgggaggccgtggcgggtggattgcctgaggtcaggagttcaagaccaacctggccaacatgatgaaaccccatctctactaaaaatccaaaaaattccccgggttttgtgggcagcccttttactcccaactttggaggggggctgaggggggggaatttgcatcccccccggacgcgggtgttccagtgagctgagatcacgccactgcactctagcctgggcaacaagagcaaaactctgtcttgaaaaaaacgaaaaggctggatgaaagctaaatttaccatttattgagtgttgacaatgtgctaggtactattatatcatctgacacttttttttttttttttttttttgagacggagtctcgctctgtcgcccaggccggactgcggactgcagtggcgcaatctcggctcactgcaagctccgcttcccgggttcacgccattctcctgcctcagcctcccgagtagctgggactacaggcgcccgctaccgcgcccggctaattttttgtatttttagtagagacggggtttcaccttgttagccaggatggtctcaatctcctgacctcatgatccacccgcctcggcctcccaaagtgctaggattacaggcgtgagccaccgcgcccggccgacacttttaatccttacaaaaaccctattagattcatattactatctgtttttatggggaaactgagactcagagagctagagtcacacaactggtaattaacagagacaagattggaaccagctgggactaactccaagtctatgctctaaactattaggcaatactgcccaaatgaaaatgattacatagtaattaagtatattaacaaaaagatgattttgtattcttactctataaatcaaaagtcatgttcaattgtggcaagtaggttttctgagagatttatcttcatgagagatttcaatagattggtctggaaaactctgtggcaactcaaggtaaagtaaaagggaggagacgcaaaaactgctttaacactaaaaatctgtaaacaaagttaatggtaatccctctattggaatggaaatcccaaatcagtaataatagctcatgaaagcaaggtgtcagaaaactgctttgccagcaaatcatttgtctgtttagagctcatcattcttttttcttttttaaatcatcacggatcaacatttttaattttttttttttttttttttttttttagatggagtctcggtctggagtgcagtggcgcgatctcggctcactgcaacctccgcctcctggattcaagcgattctcatgcctcagcctctggagtagctgggattacaggcacgcgccaccacaccgagctaatttttgtatttttagtagagacgcggtttcagcatgttggccaggatgccctgggtctcttgacctcgtgatccacccgcctctgcctcccagcgtgctgggattacaggcatgagccactgcgcccggacttttttttttttttttttttttttttttttttttttgagaccgagtttcactcttgttgcaatgacgcgatctcggctcactgcaacctccgcctcccgggttcaagcgattcccctgcctcagcctcccgagtagctgggattacaggcataagccaccatgcccggctagttttgtatttttagtagagacggggtttctctgtgttggtcaggctggtctcgaactcaggtgatccgcccgcctcggcctcccaaagtgctgggattacaggcgtgagccacagcgtccagccaacgtttttaattcttaaaactgtgtttgaatgagtgttgggtttttgttgtttggttttctttggggtaaggcggttatcaccttacacaaaaaatttacatcataatatgaaaatgcccattggttcattcaaggaaaaacataaagaatttctaacagaatggattaatggggtggaagaaatttatccattctctaaaaatttttcaaatcccaaaaacttttttttttcattttacaaaatatttacttcaaacagtagctggcattttgcatttaagagttcgaccaaaagggacagaaatataaattgctaattattagcaatggaaatgaaaatgaatcagaagtgacaataaagatttctaaaattacaagtcgatgaaaaatgaatttactgtaaattggttaacacaaatcgctgtagtaacatatctgctatgtgaaatttttaaaataaaaaccagggcggagaaggggagaaggagatctctttccaaggcccaccacccaaattctaattcttagttcacagatccaaataataataatgtgttgttctatgacttctagtattaaaagtaaatataaatttactctgcatattaaacatttacattattctaggacttctcctttaaattacgatagtacacacacaaaaaccttatttatctaattgcctttcgttggcacataaagacaaagatcgtttgcaacataatcgcgttctgatacgtttgaccctctctagtcttcgtagaaggtggccaaggtttccgttaccccggcgtccctcgggcaccgcggaaacgaaactccatcaggctccgccccacggtctgcggagtgagccaatcagggcacagcctgcgttgaccgcgtgccgggtgtcatggcggcctgcaggtactgctgctcgtgcctccggctccggc

SEQ ID NO: 2 - DNA, 410 bp, Homo Sapien
aggcccaccacccaaattctaattcttagttcacagatccaaataataataatgtgttgttctatgacttctagtattaaaagtaaatataaatttactctgcatattaaacatttacattattctaggacttctcctttaaattacgatagtacacacacaaaaaccttatttatctaattgcctttcgttggcacataaagacaaagatcgtttgcaacataatcgcgttctgatacgtttgaccctctctagtcttcgtagaaggtggccaaggtttccgttaccccggcgtccctcgggcaccgcggaaacgaaactccatcaggctccgccccacggtctgcggagtgagccaatcagggcacagcctgcgttgaccgcgtgccgggtgtcatggcggcctgcag

SEQ ID NO: 3 - DNA, 9 bp, Homo Sapien
  misc_feature (5)...(0): N = any nucleotide
gaaangaaa

SEQ ID NO: 4 - DNA, 9 bp, Homo Sapien
gaaacgaaa

SEQ ID NO: 5 - DNA, 2615 bp, Homo Sapien
gcgtgccgggtgtcatggcggcctgcaggtactgctgctcgtgcctccggctccggcccctgagcgatggtcctttccttctgccacggcgggatcgggcactcacccagttgcaagtgcgagcactatggagtagcgcagggtctcgagctgtggccgtggacttaggcaacaggaaattagaaatatcttctggaaagctggccagatttgcagatggctctgctgtagtacagtcaggtgacactgcagtaatggtcacagcggtcagtaaaacaaaaccttccccttcccagtttatgcctttggtggttgactacagacaaaaagctgctgcagcaggtagaattcccacaaactatctgagaagagaggttggtacttctgataaagaaattctaacaagtcgaataatagatcgttcaattagaccgctctttccagctggctacttctatgatacacaggttctgtgtaatctgttagcagtagatggtgtaaatgagcctgatgtcctagcaattaatggcgcttccgtagccctctcattatcagatattccttggaatggacctgttggggcagtacgaataggaataattgatggagaatatgttgttaacccaacaagaaaagaaatgtcttctagtactttaaatttagtggttgctggagcacctaaaagtcagattgtcatgttggaagcctctgcagagaacattttacagcaggacttttgccatgctatcaaagtgggagtgaaatatacccaacaaataattcagggcattcagcagttggtaaaagaaactggtgttaccaagaggacacctcagaagttatttaccccttcgccagagattgtgaaatatactcataaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattgttttgaatgaatacaaaaggtgcgatggtcgggatttgacttcacttaggaatgtaagttgtgaggtagatatgtttaaaacccttcatggatcagcattatttcaaagaggacaaacacaggtgctttgtaccgttacatttgattcattagaatctggtattaagtcagatcaagttataacagctataaatgggataaaagataaaaatttcatgctgcactacgagtttcctccttatgcaactaatgaaattggcaaagtcactggtttaaatagaagagaacttgggcatggtgctcttgctgagaaagctttgtatcctgttattcccagagattttcctttcaccataagagttacatctgaagtcctagagtcaaatgggtcatcttctatggcatctgcatgtggcggaagtttagcattaatggattcaggggttccaatttcatctgctgttgcaggcgtagcaataggattggtcaccaaaaccgatcctgagaagggtgaaatagaagattatcgtttgctgacagatattttgggaattgaagattacaatggtgacatggacttcaaaatagctggcactaataaaggaataactgcattacaggctgatattaaattacctggaataccaataaaaattgtgatggaggctattcaacaagcttcagtggcaaaaaaggagatattacagatcatgaacaaaactatttcaaaacctcgagcatctagaaaagaaaatggacctgttgtagaaactgttcaggttccattatcaaaacgagcaaaatttgttggacctggtggctataacttaaaaaaacttcaggctgaaacaggtgtaactattagtcaggtggatgaagaaacgttttctgtatttgcaccaacacccagtgttatgcatgaggcaagagacttcattactgaaatctgcaaggatgatcaggagcagcaattagaatttggagcagtatataccgccacaataactgaaatcagagatactggtgtaatggtaaaattatatccaaatatgactgcggtactgcttcataacacacaacttgatcaacgaaagattaaacatcctactgccctaggattagaagttggccaagaaattcaggtgaaatactttggacgtgacccagccgatggaagaatgaggctttctcgaaaagtgcttcagtcgccagctacaaccgtggtcagaactttgaatgacagaagtagtattgtaatgggagaacctatttcacagtcatcatctaattctcagtgattttttttttttaaagagaattctagaattctattttgtctagggtgatgtgctgtagagcaacattttagtagatcttccattgtgtagatttctatataatataaatacattttaattatttgtactaaaatgctcatttacatgtgccatttttttaattcgagtaacccatatttgtttaattgtatttacattataaatcaagaaatatttattattaaaagtaagtcatttatacatcttaga

SEQ ID NO: 6 - protein, 783 aa, Homo Sapien [sequence omitted]

SEQ ID NO: 7 - DNA, 22 bp, Homo Sapien
agttgaggggactttcccaggc

SEQ ID NO: 8 - DNA, 22 bp, Homo Sapien
agttgaggcgactttcccaggc

SEQ ID NO: 9 - DNA, 21 bp, Homo Sapien
ccacacagacagccactcacc

SEQ ID NO: 10 - DNA, 21 bp, Homo Sapien
tggcatttgtggttgggtcag

SEQ ID NO: 11 - DNA, 21 bp, Homo Sapien
ggtgcagagggttgtggagaa

SEQ ID NO: 12 - DNA, 21 bp, Homo Sapien
gcagactagggttgccagatt

SEQ ID NO: 13 - DNA, 24 bp, Homo Sapien
atggggaaggtgaaggtcggagtc

SEQ ID NO: 14 - DNA, 24 bp, Homo Sapien
gctgatgatcttgaggctgttgtc

SEQ ID NO: 15 - protein, 132 aa, Homo Sapien
GNRKLEISSGKLARFADGSAVVQSGDTAVMVTAVSKTKPSPSQFMPLVVDYRQKAAAAGRIPTNYLRREVGTSDKEILTSRIIDRSIRPLFPAGYFYDTQVLCNLLAVDGVNEPDVLAINGASVALSLSDIP

SEQ ID NO: 16 - protein, 136 aa, Homo sapiens
SLRNVSCEVDMFKTLHGSALFQRGQTQVLCTVTFDSLESGIKSDQVITAINGIKDKNFMLHYEFPPYATNEIGKVTGLNRRELGHGALAEKALYPVIPRDFPFTIRVTSEVLESNGSSSMASACGGSLALMDSGVP

SEQ ID NO: 17 - protein, 601 aa, Homo sapiens
PWNGPVGAVRIGIIDGEYVVNPTRKEMSSSTLNLVVAGAPKSQIVMLEASAENILQQDFCHAIKVGVKYTQQIIQGIQQLVKETGVTKRTPQKLFTPSPEIVKYTHKLAMERLYAVFTDYEHDKVSRDEAVNKIRLDTEEQLKEKFPEADPYEIIESFNVVAKEVFRSIVLNEYKRCDGRDLTSLRNVSCEVDMFKTLHGSALFQRGQTQVLCTVTFDSLESGIKSDQVITAINGIKDKNFMLHYEFPPYATNEIGKVTGLNRRELGHGALAEKALYPVIPRDFPFTIRVTSEVLESNGSSSMASACGGSLALMDSGVPISSAVAGVAIGLVTKTDPEKGEIEDYRLLTDILGIEDYNGDMDFKIAGTNKGITALQADIKLPGIPIKIVMEAIQQASVAKKEILQIMNKTISKPRASRKENGPVVETVQVPLSKRAKFVGPGGYNLKKLQAETGVTISQVDEETFSVFAPTPSVMHEARDFITEICKDDQEQQLEFGAVYTATITEIRDTGVMVKLYPNMTAVLLHNTQLDQRKIKHPTALGLEVGQEIQVKYFGRDPADGRMRLSRKVLQSPATTVVRTLNDRSSIVMGEPISQSSSNSQ

SEQ ID NO: 18 - protein, 490 aa, Homo sapiens
MAACRYCCSCLRLRPLSDGPFLLPRRDRALTQLQVRALWSSAGSRAVAVDLGNRKLEISSGKLARFADGSAVVQSGDTAVMVTAVSKTKPSPSQFMPLVVDYRQKAAAAGRIPTNYLRREVGTSDKEILTSRIIDRSIRPLFPAGYFYDTQVLCNLLAVDGVNEPDVLAINGASVALSLSDIPWNGPVGAVRIGIIDGEYVVMDSGVPISSAVAGVAIGLVTKTDPEKGEIEDYRLLTDILGIEDYNGDMDFKIAGTNKGITALQADIKLPGIPIKIVMEAIQQASVAKKEILQIMNKTISKPRASRKENGPVVETVQVPLSKRAKFVGPGGYNLKKLQAETGVTISQVDEETFSVFAPTPSVMHEARDFITEICKDDQEQQLEFGAVYTATITEIRDTGVMVKLYPNMTAVLLHNTQLDQRKIKHPTALGLEVGQEIQVKYFGRDPADGRMRLSRKVLQSPATTVVRTLNDRSSIVMGEPISQSSSNSQ

SEQ ID NO: 19 - protein, 507 aa, Homo sapiens
MAACRYCCSCLRLRPLSDGPFLLPRRDRALTQLQVRALWSSAGSRAVAVDLGNRKLEISSGKLARFADGSAVVQSGDTAVMVTAVSKTKPSPSQFMPLVVDYRQKAAAAGRIPTNYLRREVGTSDKEILTSRIIDRSIRPLFPAGYFYDTQVLCNLLAVDGVNEPDVLAINGASVALSLSDIPWNGPVGAVRIGIIDGEYVVNPTRKEMSSSTLNLVVAGAPKSQIVMLEASAENILQQDFCHAIKVGVKYTQQIIQGIQQLVKETGVTKRTPQKLFTPSPEIVKYTHKLAMERLYAVFTDYEHDKVSRDEAVNKIRLDTEEQLKEKFPEADPYEIIESFNVVAKEVFRSIVLNEYKRCDGRDLTSLRNVSCEVDMFKTLHGSALFQRGQTQVLCTVTFDSLESGIKSDQVITAINGIKDKNFMLHYEFPPYATNEIGKVTGLNRRELGHGALAEKALYPVIPRDFPFTIRVTSEVLESNGSSSMASACGGSLALMDSGVPISSAVA

SEQ ID NO: 20 - protein, 45 aa, Homo sapiens
MAACRYCCSCLRLRPLSDGPFLLPRRDRALTQLQVRALWSSAGSR

SEQ ID NO: 21 - protein, 75 aa, Homo sapiens
KLAMERLYAVFTDYEHDKVSRDEAVNKIRLDTEEQLKEKFPEADPYEIIESFNVVAKEVFRSIVLNEYKRCDGRD

SEQ ID NO: 22 - protein, 63 aa, Homo sapiens
PVVETVQVPLSKRAKFVGPGGYNLKKLQAETGVTISQVDEETFSVFAPTPSVMHEARDFITEI

SEQ ID NO: 23 - protein, 75 aa, Homo sapiens
LEFGAVYTATITEIRDTGVMVKLYPNMTAVLLHNTQLDQRKIKHPTALGLEVGQEIQVKYFGRDPADGRMRLSRK

SEQ ID NO: 24 - DNA, 396 bp, Homo sapiens
ggcaacaggaaattagaaatatcttctggaaagctggccagatttgcagatggctctgctgtagtacagtcaggtgacactgcagtaatggtcacagcggtcagtaaaacaaaaccttccccttcccagtttatgcctttggtggttgactacagacaaaaagctgctgcagcaggtagaattcccacaaactatctgagaagagaggttggtacttctgataaagaaattctaacaagtcgaataatagatcgttcaattagaccgctctttccagctggctacttctatgatacacaggttctgtgtaatctgttagcagtagatggtgtaaatgagcctgatgtcctagcaattaatggcgcttccgtagccctctcattatcagatattcct

SEQ ID NO: 25 - DNA, 408 bp, Homo sapiens
tcacttaggaatgtaagttgtgaggtagatatgtttaaaacccttcatggatcagcattatttcaaagaggacaaacacaggtgctttgtaccgttacatttgattcattagaatctggtattaagtcagatcaagttataacagctataaatgggataaaagataaaaatttcatgctgcactacgagtttcctccttatgcaactaatgaaattggcaaagtcactggtttaaatagaagagaacttgggcatggtgctcttgctgagaaagctttgtatcctgttattcccagagattttcctttcaccataagagttacatctgaagtcctagagtcaaatgggtcatcttctatggcatctgcatgtggcggaagtttagcattaatggattcaggggttcca

SEQ ID NO: 26 - DNA, 1803 bp, Homo sapiens
ccttggaatggacctgttggggcagtacgaataggaataattgatggagaatatgttgttaacccaacaagaaaagaaatgtcttctagtactttaaatttagtggttgctggagcacctaaaagtcagattgtcatgttggaagcctctgcagagaacattttacagcaggacttttgccatgctatcaaagtgggagtgaaatatacccaacaaataattcagggcattcagcagttggtaaaagaaactggtgttaccaagaggacacctcagaagttatttaccccttcgccagagattgtgaaatatactcataaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattgttttgaatgaatacaaaaggtgcgatggtcgggatttgacttcacttaggaatgtaagttgtgaggtagatatgtttaaaacccttcatggatcagcattatttcaaagaggacaaacacaggtgctttgtaccgttacatttgattcattagaatctggtattaagtcagatcaagttataacagctataaatgggataaaagataaaaatttcatgctgcactacgagtttcctccttatgcaactaatgaaattggcaaagtcactggtttaaatagaagagaacttgggcatggtgctcttgctgagaaagctttgtatcctgttattcccagagattttcctttcaccataagagttacatctgaagtcctagagtcaaatgggtcatcttctatggcatctgcatgtggcggaagtttagcattaatggattcaggggttccaatttcatctgctgttgcaggcgtagcaataggattggtcaccaaaaccgatcctgagaagggtgaaatagaagattatcgtttgctgacagatattttgggaattgaagattacaatggtgacatggacttcaaaatagctggcactaataaaggaataactgcattacaggctgatattaaattacctggaataccaataaaaattgtgatggaggctattcaacaagcttcagtggcaaaaaaggagatattacagatcatgaacaaaactatttcaaaacctcgagcatctagaaaagaaaatggacctgttgtagaaactgttcaggttccattatcaaaacgagcaaaatttgttggacctggtggctataacttaaaaaaacttcaggctgaaacaggtgtaactattagtcaggtggatgaagaaacgttttctgtatttgcaccaacacccagtgttatgcatgaggcaagagacttcattactgaaatctgcaaggatgatcaggagcagcaattagaatttggagcagtatataccgccacaataactgaaatcagagatactggtgtaatggtaaaattatatccaaatatgactgcggtactgcttcataacacacaacttgatcaacgaaagattaaacatcctactgccctaggattagaagttggccaagaaattcaggtgaaatactttggacgtgacccagccgatggaagaatgaggctttctcgaaaagtgcttcagtcgccagctacaaccgtggtcagaactttgaatgacagaagtagtattgtaatgggagaacctatttcacagtcatcatctaattctcag

SEQ ID NO: 27 - DNA, 1470 bp, Homo sapiens
atggcggcctgcaggtactgctgctcgtgcctccggctccggcccctgagcgatggtcctttccttctgccacggcgggatcgggcactcacccagttgcaagtgcgagcactatggagtagcgcagggtctcgagctgtggccgtggacttaggcaacaggaaattagaaatatcttctggaaagctggccagatttgcagatggctctgctgtagtacagtcaggtgacactgcagtaatggtcacagcggtcagtaaaacaaaaccttccccttcccagtttatgcctttggtggttgactacagacaaaaagctgctgcagcaggtagaattcccacaaactatctgagaagagaggttggtacttctgataaagaaattctaacaagtcgaataatagatcgttcaattagaccgctctttccagctggctacttctatgatacacaggttctgtgtaatctgttagcagtagatggtgtaaatgagcctgatgtcctagcaattaatggcgcttccgtagccctctcattatcagatattccttggaatggacctgttggggcagtacgaataggaataattgatggagaatatgttgttatggattcaggggttccaatttcatctgctgttgcaggcgtagcaataggattggtcaccaaaaccgatcctgagaagggtgaaatagaagattatcgtttgctgacagatattttgggaattgaagattacaatggtgacatggacttcaaaatagctggcactaataaaggaataactgcattacaggctgatattaaattacctggaataccaataaaaattgtgatggaggctattcaacaagcttcagtggcaaaaaaggagatattacagatcatgaacaaaactatttcaaaacctcgagcatctagaaaagaaaatggacctgttgtagaaactgttcaggttccattatcaaaacgagcaaaatttgttggacctggtggctataacttaaaaaaacttcaggctgaaacaggtgtaactattagtcaggtggatgaagaaacgttttctgtatttgcaccaacacccagtgttatgcatgaggcaagagacttcattactgaaatctgcaaggatgatcaggagcagcaattagaatttggagcagtatataccgccacaataactgaaatcagagatactggtgtaatggtaaaattatatccaaatatgactgcggtactgcttcataacacacaacttgatcaacgaaagattaaacatcctactgccctaggattagaagttggccaagaaattcaggtgaaatactttggacgtgacccagccgatggaagaatgaggctttctcgaaaagtgcttcagtcgccagctacaaccgtggtcagaactttgaatgacagaagtagtattgtaatgggagaacctatttcacagtcatcatctaattctcag

SEQ ID NO: 28 - DNA, 1521 bp, Homo sapiens
atggcggcctgcaggtactgctgctcgtgcctccggctccggcccctgagcgatggtcctttccttctgccacggcgggatcgggcactcacccagttgcaagtgcgagcactatggagtagcgcagggtctcgagctgtggccgtggacttaggcaacaggaaattagaaatatcttctggaaagctggccagatttgcagatggctctgctgtagtacagtcaggtgacactgcagtaatggtcacagcggtcagtaaaacaaaaccttccccttcccagtttatgcctttggtggttgactacagacaaaaagctgctgcagcaggtagaattcccacaaactatctgagaagagaggttggtacttctgataaagaaattctaacaagtcgaataatagatcgttcaattagaccgctctttccagctggctacttctatgatacacaggttctgtgtaatctgttagcagtagatggtgtaaatgagcctgatgtcctagcaattaatggcgcttccgtagccctctcattatcagatattccttggaatggacctgttggggcagtacgaataggaataattgatggagaatatgttgttaacccaacaagaaaagaaatgtcttctagtactttaaatttagtggttgctggagcacctaaaagtcagattgtcatgttggaagcctctgcagagaacattttacagcaggacttttgccatgctatcaaagtgggagtgaaatatacccaacaaataattcagggcattcagcagttggtaaaagaaactggtgttaccaagaggacacctcagaagttatttaccccttcgccagagattgtgaaatatactcataaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattgttttgaatgaatacaaaaggtgcgatggtcgggatttgacttcacttaggaatgtaagttgtgaggtagatatgtttaaaacccttcatggatcagcattatttcaaagaggacaaacacaggtgctttgtaccgttacatttgattcattagaatctggtattaagtcagatcaagttataacagctataaatgggataaaagataaaaatttcatgctgcactacgagtttcctccttatgcaactaatgaaattggcaaagtcactggtttaaatagaagagaacttgggcatggtgctcttgctgagaaagctttgtatcctgttattcccagagattttcctttcaccataagagttacatctgaagtcctagagtcaaatgggtcatcttctatggcatctgcatgtggcggaagtttagcattaatggattcaggggttccaatttcatctgctgttgca

SEQ ID NO: 29 - DNA, 135 bp, Homo sapiens
atggcggcctgcaggtactgctgctcgtgcctccggctccggcccctgagcgatggtcctttccttctgccacggcgggatcgggcactcacccagttgcaagtgcgagcactatggagtagcgcagggtctcga

SEQ ID NO: 30 - DNA, 225 bp, Homo sapiens
aaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattgttttgaatgaatacaaaaggtgcgatggtcgggat

SEQ ID NO: 31 - DNA, 189 bp, Homo sapiens
cctgttgtagaaactgttcaggttccattatcaaaacgagcaaaatttgttggacctggtggctataacttaaaaaaacttcaggctgaaacaggtgtaactattagtcaggtggatgaagaaacgttttctgtatttgcaccaacacccagtgttatgcatgaggcaagagacttcattactgaaatc

SEQ ID NO: 32 - DNA, 225 bp, Homo sapiens
ttagaatttggagcagtatataccgccacaataactgaaatcagagatactggtgtaatggtaaaattatatccaaatatgactgcggtactgcttcataacacacaacttgatcaacgaaagattaaacatcctactgccctaggattagaagttggccaagaaattcaggtgaaatactttggacgtgacccagccgatggaagaatgaggctttctcgaaaa

SEQ ID NO: 33 - DNA, 24 bp, Artificial Sequence
  Synthetic Sense Primer
gctagcatggcggcctgcaggtac

SEQ ID NO: 34 - DNA, 54 bp, Artificial Sequence
  Synthetic AntiSense Primer
ggatcctcaagcgtaatctggaacatcgtatgggtactgagaattagatgatga

SEQ ID NO: 35 - DNA, 30 bp, Artificial Sequence
  Synthetic Sense Primer
gctagcatgccttggaatggacctgttggg

SEQ ID NO: 36 - DNA, 27 bp, Artificial Sequence
  Synthetic Sense Primer
gttaacatggattcaggggttccaatt

SEQ ID NO: 37 - DNA, 27 bp, Artificial Sequence
  Synthetic Sense Primer
gctagcatggattcaggggttccaatt

SEQ ID NO: 38 - DNA, 58 bp, Artificial Sequence
  Synthetic Primer
ggatcctcaagcgtaatctggaacatcgtatgggtactgcaacagcagatgaaattgg

SEQ ID NO: 39 - protein, 9 aa, Artificial Sequence
  Synthetic C-terminal Hemagglutinin (HA)-epitope
YPYDVPDYA

SEQ ID NO: 40 - DNA, 21 bp, Artificial Sequence
  Synthetic Control Sequence
aacaaaaccttccccttccca

SEQ ID NO: 41 - DNA, 23 bp, Artificial Sequence
  Synthetic Control Sequence
aagggtcgtctatagggatcgat